(19) United States
(12) Patent Application Publication
(10) Pub. No.: US 2022/0362264 A1
(43) Pub. Date: Nov. 17, 2022

(54) ABIRATERONE-CYCLIC OLIGOMER PHARMACEUTICAL FORMULATIONS AND METHODS OF FORMATION AND ADMINISTRATION THEREOF

(71) Applicants: DISPERSOL TECHNOLOGIES, LLC., Georgetown, TX (US); BOARD OF REGENTS, THE UNIVERSITY OF TEXAS SYSTEM, Austin, TX (US)

(72) Inventors: Dave MILLER, Georgetown, TX (US); Urvi GALA, Austin, TX (US); Robert O. WILLIAMS, III, Austin, TX (US); Angela SPANGENBERG, Austin, TX (US)

(73) Assignees: DISPERSOL TECHNOLOGIES, LLC., Georgetown, TX (US); BOARD OF REGENTS, THE UNIVERSITY OF TEXAS SYSTEM, Austin, TX (US)

(21) Appl. No.: 17/437,144

(22) PCT Filed: Mar. 17, 2020
(86) PCT No.: PCT/US2020/023066

§ 371 Date: Sep. 8, 2021

Related U.S. Application Data
(60) Provisional application No. 62/820,076, filed on Mar. 18, 2019.
(60) Provisional application No. 62/942,111, filed on Nov. 30, 2019.

(51) Int. Cl. A61K 31/58; A61K 9/20

(52) U.S. Cl. CPC A61K 31/58; A61K 9/205; A61K 9/2054

(57) ABSTRACT

The present disclosure relates to pharmaceutical formulations including abiraterone and a cyclic oligomer, as well as tablets including such pharmaceutical formulations, methods of forming such pharmaceutical formulations, and methods of administering such pharmaceutical formulations or tablets.

### PRIORITY CLAM

This application claims benefit of priority to U.S. Provisional Application Ser. No. 62/820,076, filed on Mar. 18, 2019, and U.S. Provisional Application Ser. No. 62/942,111, filed on Nov. 30, 2019, the entire contents of both applications being hereby incorporated by reference.

### TECHNICAL FIELD

The present disclosure relates to abiraterone pharmaceutical formulations and methods of forming and administering such pharmaceutical formulations.

### BACKGROUND

Certain types of advanced prostate cancer are often difficult to treat because cancer cell growth is driven by androgens. Androgens are made primarily by the testes in adult males, but they are also produced by the adrenal glands and, in the case of some prostate cancers, by the cancer cells themselves. As a result, some advanced prostate cancers continue to exhibit androgen-induced growth even after castration of the patient. Abiraterone blocks androgen production, and particular testosterone production in the testes, adrenal glands, and cancer cells themselves. Accordingly, orally administered abiraterone acetate has been approved for use in patients with metastatic castration-resistant prostate cancer (mCRPC). In addition, abiraterone has shown potential efficacy in the treatment of other androgen sensitive cancers, e.g., breast cancer.

Abiraterone blocks androgen biosynthesis by inhibiting Cytochrome P450 17A1 (CYP17A1). As a result, patients taking abiraterone may experience general negative effects of insufficient glucocorticoid levels, such as low serum cortisol and a compensatory increase in adrenocorticotropic hormone. Patients taking abiraterone are, therefore, typically also given glucocorticoid replacement therapy.

Abiraterone is highly lipophilic and has low aqueous solubility in the gastrointestinal tract, thus severely limiting the drug's oral bioavailability. The leading commercial product, Zytiga, mitigates this insolubility issue by use of the more soluble ester prodrug, abiraterone acetate. However, the effectiveness of the prodrug toward improving bioavailability is limited, as evidenced by the food effect and pharmacokinetic variability cited in the label. Specifically, the 10-fold increase in AUC when Zytiga is administered with a high fat meal suggests that the absolute bioavailability of abiraterone is maximally 10% when Zytiga is administered per the label (fasted). Further, exposure was not significantly increased when the Zytiga dose was doubled from 1,000 to 2,000 mg (8% increase in the mean AUC). The results of this study imply that Zytiga is dosed near the absorption limit.

In the treatment of metastatic castration resistant prostate cancer with abiraterone, reductions in prostate specific antigen (PSA) are predictive of improved clinical outcomes. In well controlled trials, dosing with 1,000 mg abiraterone acetate daily only achieves target PSA reductions in up to 60% of treated patients. Thus, abiraterone remains a difficult drug to administer optimally.

Additionally, recent findings have suggested that abiraterone response in patients with metastatic castration-resistant prostate cancer is correlated to steady state trough levels (Cₘᵢₙ) (Xu et al., Clin. Pharmacokinet. 56: 55-63, 2017) Specifically, Cₘᵢₙ values greater than about 30 ng/mL correlate with greater PSA decay rate, suggesting that improved abiraterone bioavailability and optimizing the pharmacokinetic profile would lead to better therapeutic efficacy, i.e., anti-tumor response. This finding indicates that the therapeutic benefit of Zytiga is limited by sub-optimal abiraterone delivery and highlights a critical need for improved abiraterone compositions, specifically those that can increase systemic abiraterone exposure and trough levels.

### SUMMARY

The present disclosure provides a pharmaceutical formulation including abiraterone and a cyclic oligomer excipient.

According to various further embodiments of the pharmaceutical formulation, which may all be combined with one another unless clearly mutually exclusive:

i) the abiraterone may include amorphous abiraterone;

i-a) the abiraterone may contain less than 5% crystalline material, less than 1% crystalline material, or no crystalline material;

ii) the abiraterone may include at least 99% abiraterone;

iii) the abiraterone may include at least 99% abiraterone, having the structural formula:

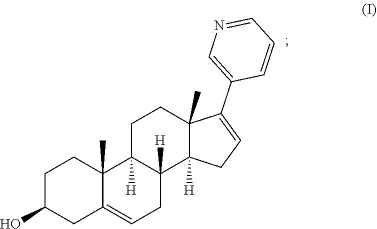

iv) the abiraterone may include at least 99% abiraterone salt;

v) the abiraterone may include at least 99% abiraterone ester;

v-a) the abiraterone ester may include abiraterone acetate, having the structural formula:

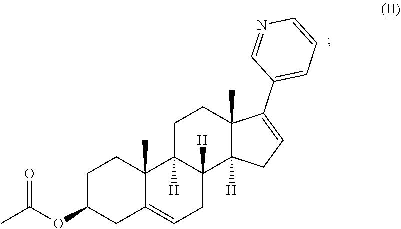

vi) the abiraterone may include at least 99% abiraterone solvate;

vii) the abiraterone may include at least 99% abiraterone hydrate;

viii) the pharmaceutical formulation may include 10 mg, 25 mg, 50 mg, 70 mg, 100 mg, or 250 mg of amorphous abiraterone;

ix) the pharmaceutical formulation may include an amount of amorphous abiraterone or a salt thereof sufficient to achieve the same or greater therapeutic effect, bioavailability, Cₘᵢₙ, Cₘₐₓ or Tₘₐₓ in a patient as 50 mg, 70 mg, 100 mg, 250 mg, 500 mg, or 1000 mg of crystalline abiraterone or crystalline abiraterone acetate when consumed on an empty stomach;

x) the pharmaceutical formulation may include an amount of 10 mg, 25 mg, 50 mg, 70 mg, 100 mg, 250 mg or 500 mg of amorphous abiraterone or a salt thereof;

xi) the pharmaceutical formulation may include an amount of amorphous abiraterone or a salt thereof sufficient to achieve the same or greater therapeutic effect, bioavailability, Cₘᵢₙ, Cₘₐₓ or Tₘₐₓ in a patient as 10 mg, 25 mg, 50 mg, 70 mg, 100 mg, 250 mg, 500 mg or 1000 mg of crystalline abiraterone or crystalline abiraterone acetate when consumed on an empty stomach;

xii) the pharmaceutical formulation may include 1,000 mg of amorphous abiraterone or a salt thereof;

xiii) the pharmaceutical formulation may include an amount of amorphous abiraterone or a salt thereof sufficient to achieve the same or greater therapeutic effect, bioavailability, Cₘᵢₙ, Cₘₐₓ or Tₘₐₓ in a patient as 1,000 mg of crystalline abiraterone or crystalline abiraterone acetate when consumed on an empty stomach;

xiv) the abiraterone or a salt thereof and cyclic oligomer may be present in a molar ratio of 1:0.25 to 1:25;

xv) the abiraterone or a salt thereof and cyclic oligomer may be present in a molar ratio of at least 1:2;

xvi) the pharmaceutical formulation may include 1% to 50% by weight amorphous abiraterone or a salt thereof;

xvii) the pharmaceutical formulation may include at least 10% by weight amorphous abiraterone or a salt thereof;

xviii) the cyclic oligomer excipient may include a cyclic oligosaccharide or cyclic oligosaccharide derivative;

xvii-a) the cyclic oligosaccharide or cyclic oligosaccharide derivative may include a cyclodextrin or a cyclodextrin derivative;

xvii-a-a) the cyclodextrin derivative may include a hydroxy propyl β cyclodextrin;

xvii-a-b) the cyclodextrin derivative may include a sodium (Na) sulfo-butyl ether β cyclodextrin;

xvii-a-c) the cyclodextrin derivative may include a hydroxypropyl group

xvii-a-d) the cyclodextrin derivative may include a sulfo-butyl ether functional group;

xvii-a-e) the cyclodextrin derivative may include a methyl group;

xvii-a-f) the cyclodextrin derivative may include a carboxymethyl group;

xix) the pharmaceutical formulation may include 50% to 99% by weight cyclic oligomer excipient;

xx) the pharmaceutical formulation may include at least 90% by weight cyclic oligomer excipient;

xxi) the pharmaceutical formulation may include an additional excipient;

xxi-a) the cyclic oligomer excipient may be a primary excipient;

xxi-b) the additional excipient may be the primary excipient;

xxi-b-a) the additional excipient may be a secondary excipient;

xxi-c) the additional excipient may be a polymer excipient;

xxi-c-a) the polymer excipient may be water soluble;

xxi-c-b) the polymer excipient may include a non-ionic polymer;

xxi-c-c) the polymer excipient may include an ionic polymer;

xxi-c-d) the polymer excipient may include a hydroxy propyl methyl cellulose acetate succinate;

xxi-c-d-a) the hydroxypropylmethyl cellulose acetate succinate may have 5-14% acetate substitution and 4-18% succinate substitution;

xxi-c-d-a-a) the hydroxypropylmethyl cellulose acetate succinate may have 10-14% acetate substitution and 4-8% succinate substitution;

xxi-c-d-a-a-a) the hydroxypropylmethyl cellulose acetate succinate may have 12% acetate substitution and 6% succinate substitution;

xxi-d) the pharmaceutical formulation may include between 1% and 49% by weight additional excipient;

xxi-e) the pharmaceutical formulation may include 10% by weight or less additional excipient;

xxii) the pharmaceutical formulation may include a glucocorticoid replacement API;

xxii-a) the glucocorticoid replacement API may include prednisone, methylprednisone, prednisolone, methylprednisolone, dexamethasone, or a combination thereof;

xxiii) the pharmaceutical formulation may include an amount of amorphous abiraterone or a salt thereof sufficient to achieve at least a 6-fold increase in Cₘₐₓ in a patient as an equivalent amount of crystalline abiraterone or crystalline abiraterone acetate when consumed on an empty stomach;

xxiv) the pharmaceutical formulation may include an amount of amorphous abiraterone or a salt thereof sufficient to achieve at least a 3-fold increase in AUCo₀₋ₜ in a patient as an equivalent amount of crystalline abiraterone or crystalline abiraterone acetate when consumed on an empty stomach;

xxv) the pharmaceutical formulation may include an amount of amorphous abiraterone or a salt thereof sufficient to achieve at least a 6% decrease in variability of Cₘₐₓ in a patient as an equivalent amount of crystalline abiraterone or crystalline abiraterone acetate when consumed on an empty stomach;

xxvi) the pharmaceutical formulation may include an amount of amorphous abiraterone or a salt thereof sufficient to achieve at least a 4% decrease in variability of AUC₀₋ₜ in a patient as an equivalent amount of crystalline abiraterone or crystalline abiraterone acetate when consumed on an empty stomach;

xxvii) the pharmaceutical formulation may include an amount of amorphous abiraterone or a salt thereof sufficient to achieve at least a 25% decrease in variability of Cₘₐₓ in a patient when consumed in a fed state as an equivalent amount of crystalline abiraterone or crystalline abiraterone acetate when consumed on an empty stomach;

xxviii) the pharmaceutical formulation may include an amount of amorphous abiraterone or a salt thereof sufficient to achieve at least a 23% decrease in variability of AUC₀ₜ as in a patient when consumed in a fed state as an equivalent amount of crystalline abiraterone or crystalline abiraterone acetate when consumed on an empty stomach;

xxix) the pharmaceutical formulation may include an amount of amorphous abiraterone or a salt thereof sufficient to achieve less than 30% variation in Cₘₐₓ when consumed in a fed state as compared to Cmax when consumed in a fasted state;

xxx) the pharmaceutical formulation may include an amount of amorphous abiraterone or a salt thereof sufficient to achieve less than 10% variation in AUC₀₋ₜ in a patient when consumed in a fed state as compared to AUC₀₋ₜ when consumed in a fasted state;

xxxi) the pharmaceutical formulation may include an amount of amorphous abiraterone or a salt thereof sufficient to achieve a mean Cₘᵢₙ of least 35 ng/mL in a population of human patients, when administered once daily, twice daily, three times daily or four times daily.

xxxii) the pharmaceutical formulation may include an amount of amorphous abiraterone or a salt thereof sufficient to achieve increased therapeutic effect, bioavailability, Cmin, or Cmax in a patient with metastatic castration-resistant prostate cancer and primary resistance as compared to an equivalent or greater amount of crystalline abiraterone or an equivalent or greater amount of crystalline abiraterone acetate when consumed in a fasted state;

xxxiii) the pharmaceutical formulation may include an amount of amorphous abiraterone or a salt thereof sufficient to achieve increased therapeutic effect, bioavailability, Cmin, or Cmax in a patient with metastatic castration-resistant prostate cancer and acquired resistance as compared to an equivalent or greater amount of crystalline abiraterone or an equivalent or greater amount of crystalline abiraterone acetate when consumed in a fasted state;

xxxiv) the pharmaceutical formulation may include an amount of amorphous abiraterone or a salt thereof sufficient to achieve increased therapeutic effect, bioavailability, Cmin, or Cmax in a patient with triple-negative breast cancer as compared to an equivalent or greater amount of crystalline abiraterone or an equivalent or greater amount of crystalline abiraterone acetate when consumed in a fasted state;

xxxv) the pharmaceutical formulation may include an amount of amorphous abiraterone or a salt thereof sufficient to achieve increased therapeutic effect, bioavailability, Cmin, or Cmax in a patient with non-metastatic castration-resistant prostate cancer as compared to an equivalent or greater amount of crystalline abiraterone or an equivalent or greater amount of crystalline abiraterone acetate when consumed in a fasted state.

xxxvi) the pharmaceutical formulation may include an inclusion complex including amorphous abiraterone and the cyclic oligomer excipient.

xxxvi-a) the pharmaceutical formulation including the inclusion complex may include up to 30%, up to 20%, or up to 10% by weight amorphous abiraterone.

xxxvi-b) the pharmaceutical formulation including the inclusion complex may include up to 10% by weight amorphous abiraterone.

xxxvi-c) the pharmaceutical formulation including the inclusion complex may be formed by a method that includes thermokinetic compounding.

xxxvi-d) at least 90%, 91%, 92%, 93%, 94%, 95%, 96%, 97%, 98%, 99%, or 99.9% of the amorphous abiraterone may be present in the inclusion complex.

xxxvi-e) in response to heating the pharmaceutical formulation to a temperature up to 90% of the melting point of a crystalline form of abiraterone, and allowing the pharmaceutical formulation to cool to room temperature, less than 10%, 9%, 8%, 7%, 6%, 5%, 4%, 3%, 2%, 1%, or 0.1% of the abiraterone may be in crystalline form.

xxxvi-e-i) the less than 10%, 9%, 8%, 7%, 6%, 5%, 4%, 3%, 2%, 1%, or 0.1% of the abiraterone in crystalline form may be determined by a method comprising X-ray diffraction.

xxxvi-f) the pharmaceutical formulation including the inclusion complex may have at least 13-17-fold increased dissolution in a gastro-intestinal tract of a patient than a pharmaceutical formulation containing neat crystalline abiraterone, as evidenced by dissolution in at least one of 0.01 N HCl and biorelevant media selected from Simulated Gastric Fluid (SGF), Fasted State Simulated Intestinal Fluid (FaSSIF), and Fed State Simulated Intestinal Fluid (FeSSIF).

xxxvi-g) the pharmaceutical formulation including the inclusion complex may have has up to 4-fold increase in bioavailability of abiraterone in a patient as compared to a greater amount of crystalline abiraterone or crystalline abiraterone acetate, when consumed on an empty stomach.

The disclosure further provides a tablet for oral administration, which may include any pharmaceutical formulation above or otherwise described herein.

According to various further embodiments of the tablet, which may all be combined with one another unless clearly mutually exclusive:

i) the tablet may include a coating;

i-a) the coating may include a glucocorticoid replacement API;

i-a-a) the glucocorticoid replacement API may include prednisone, methylprednisone, prednisolone, methylprednisolone, dexamethasone, or a combination thereof;

ii) the tablet may include an external phase including an additional amount of the cyclic oligomer excipient;

iii) the tablet may include an external phase including at least one additional excipient;

iv) the tablet may include a concentration enhancing polymer;

iv-a) the concentration enhancing polymer may include a hydroxypropylmethyl cellulose acetate succinate.

v) the tablet may include an external phase including one or more water swellable polymers

v-a) the water swellable polymers may include polyethylene oxide, hydroxymethyl cellulose, hydroxyethyl cellulose, hydroxypropyl cellulose, hydroxypropylmethyl cellulose, and carboxymethyl cellulose

v-b) the tablet may be of a geometry such that when the water swellable polymers are hydrated the size and shape of the tablet prevents passage of the tablet through the pylorus of the stomach

v-c) the tablet may have drug release profile such as immediate release, or modified release such as extended release which may be sustained release or controlled release, or pulsatile release or delayed release

The tablet may include an external phase including at least one additional drug release modifying excipient or may include an external phase including of one or more hydrogel forming excipient, or may include an external phase including of combination of polyethylene oxide and hydroxypropyl methyl cellulose.

The present disclosure also provides a method of forming a pharmaceutical formulation by compounding crystalline abiraterone or a salt thereof and a cyclic oligomer excipient in a thermokinetic mixer at a temperature less than or equal to 200° C. for less than 300 seconds to form an amorphous abiraterone and a cyclic oligomer excipient.

According to various further embodiments of the method, which may all be combined with one another unless clearly mutually exclusive:

i) the pharmaceutical formulation may be any pharmaceutical formulation above or otherwise described herein;

ii) the method may also include compounding at least one additional excipient with the crystalline abiraterone and cyclic oligomer excipient;

iii) compounding in the thermokinetic mixer may not cause substantial thermal degradation of the abiraterone or a salt thereof;

iv) compounding in the thermokinetic mixer may not cause substantial thermal degradation of the cyclic oligomer excipient;

v) compounding in the thermokinetic mixer may not cause substantial thermal degradation of the additional excipient.

The present disclosure also provides a method of forming a pharmaceutical formulation, by hot-melt extrusion processing crystalline abiraterone or a salt thereof and a cyclic oligomer excipient to form an amorphous abiraterone and the cyclic oligomer excipient in which the abiraterone is not substantially thermally degraded.

According to various further embodiments of the method, which may all be combined with one another unless clearly mutually exclusive:

i) the pharmaceutical formulation may be any pharmaceutical formulation above or otherwise described herein;

ii) the method may also include processing at least one additional excipient with the crystalline abiraterone and cyclic oligomer excipient;

iii) melt processing may not cause substantial thermal degradation of the cyclic oligomer excipient;

iv) melt processing may not cause substantial thermal degradation of the additional excipient.

The present disclosure further provides a method of forming a pharmaceutical formulation including dissolving crystalline abiraterone or a salt thereof and a cyclic oligomer excipient in a common organic solvent to form a dissolved mixture and spray drying the dissolved mixture to form an amorphous abiraterone and cyclic oligomer excipient.

According to various further embodiments of the method, which may all be combined with one another unless clearly mutually exclusive:

i) the pharmaceutical formulation may be any pharmaceutical formulation above or otherwise described herein;

ii) the method may further include dissolving at least one additional excipient with the crystalline abiraterone and cyclic oligomer excipient and spray drying;

iii) spray drying may not cause substantial thermal degradation of the abiraterone;

iv) spray drying may not cause substantial thermal degradation of the cyclic oligomer excipient;

v) spray drying may not cause substantial thermal degradation of the additional excipient.

The present disclosure also includes a method of forming a pharmaceutical formulation, the method including combining abiraterone and a cyclic oligomer excipient by a method including wet mass extrusion, high intensity mixing, high intensity mixing with a solvent, ball milling, or ball milling with a solvent to firm an amorphous abiraterone and cyclic oligomer excipient.

The present disclosure also includes any pharmaceutical formulations prepared according to any of the above methods, which may also have any of the other features of pharmaceutical formulations described above or otherwise herein.

The present disclosure also includes tablets containing any pharmaceutical formulations prepared according to any of the above methods, which may also have any of the other features of pharmaceutical formulations or tablets described above or otherwise herein.

The present disclosure also provides a method of treating prostate cancer in a patient by administering any pharmaceutical formulation described above or otherwise herein or any tablet described above or otherwise herein to a patient having prostate cancer.

According to various further embodiments of the method, which may all be combined with one another unless clearly mutually exclusive:

i) the patient may have castration-resistant prostate cancer, metastatic castration-resistant prostate cancer, metastatic prostate cancer, locally advanced prostate cancer, relapsed prostate cancer, non-metastatic castration-resistant prostate cancer, or other high-risk prostate cancer;

ii) the patient may have previously received treatment with chemotherapy;

ii-a) the chemotherapy may include docetaxel;

iii) the patient may have previously received treatment with enzalutamide;

iv) the patient may have previously experienced a sub-optimal response to crystalline abiraterone acetate;

v) the pharmaceutical formulation or tablet may be administered to the patient in combination with androgen-deprivation therapy;

vi) the pharmaceutical formulation or tablet may be administered to the patient in combination with a glucocorticoid replacement API;

vii) the pharmaceutical formulation or tablet may be administered once daily;

viii) the pharmaceutical formulation or tablet may be administered twice daily, three times daily, four times daily or more;

ix) the pharmaceutical formulation or tablet may include amorphous abiraterone or a salt thereof and may be administered at dose lower in weight of abiraterone as compared to a dose in weight of abiraterone acetate sufficient to achieve an equivalent or higher therapeutic effect, bioavailability, Cₘᵢₙ, Cₘₐₓ or Tₘₐₓ,

x) the patient may have metastatic castration-resistant prostate cancer and primary resistance to treatment with crystalline abiraterone or crystalline abiraterone acetate;

xi) the patient may have metastatic castration-resistant prostate cancer and acquired resistance to treatment with crystalline abiraterone or crystalline abiraterone acetate.

The present disclosure also provides a method of treating various androgen sensitive cancers by administering any pharmaceutical formulation described above or otherwise herein or any tablet described above or otherwise herein to a patient having an androgen sensitive cancer.

According to various further embodiments of the method, which may all be combined with one another unless clearly mutually exclusive:

i. the patient may have breast cancer or triple-negative androgen receptor positive locally advanced/metastatic breast cancer or ER-positive HER2-negative breast cancer or ER positive metastatic breast cancer or apocrine breast cancer;

ii. the patient may have Cushing's syndrome with adrenocortical carcinoma;

iii. the patient may have urothelial carcinoma or bladder cancer or urinary bladder neoplasms;

iv. the patient may have androgen receptor expressing, relapsed/metastatic, salivary gland cancer or recurrent and/or metastatic salivary gland cancer or salivary glands tumors or salivary duct carcinoma;

v. the patient may have previously received treatment with chemotherapy;
iv-a) the chemotherapy may include docetaxel;

vi. the patient may have previously received treatment with medication used for breast cancer, adrenal carcinoma and salivary gland cancer;

vii. the patient may have previously experienced a sub-optimal response to crystalline abiraterone acetate;

viii. the pharmaceutical formulation or tablet may be administered to the patient in combination with androgen-deprivation therapy;

ix. the pharmaceutical formulation or tablet may be administered to the patient in combination with a glucocorticoid replacement API;

x. the pharmaceutical formulation or tablet may be administered once daily;

xi. the pharmaceutical formulation or tablet may be administered twice daily, three times daily, four times daily or more;

xii. the pharmaceutical formulation or tablet may include amorphous abiraterone and may be administered at dose lower in weight of abiraterone as compared to a dose in weight of abiraterone acetate sufficient to achieve an equivalent or higher therapeutic effect, bioavailability, Cₘᵢₙ, Cₘₐₓ or Tₘₐₓ.

It is contemplated that any method or composition described herein can be implemented with respect to any other method or composition described herein.

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

Unless it is otherwise clear that a single entity is intended, terms such as “a,” “an,” and “the” are not intended to refer to only a singular entity and include the general class of which a specific example is described for illustration.

In addition, unless it is clear that a precise value is intended, numbers recited herein should be interpreted to include variations above and below that number that may achieve substantially the same results as that number, or variations that are “about” the same number.

Finally, a derivative of the present disclosure may include a chemically modified molecule that has an addition, removal, or substitution of a chemical moiety of the parent molecule.

### BRIEF DESCRIPTION OF THE DRAWINGS

The present disclosure may be further understood through reference to the attached figures in combination with the detailed description that follows.

FIG. 1 is an exemplary X-ray diffractogram of neat crystalline abiraterone.

FIG. 2 is a set of exemplary X-ray diffractograms of abiraterone solid dispersions with various polymer excipients. Excipient type (cellulose-based, polyvinyl-based, or acrylate-based) is indicated.

FIG. 3 is an exemplary X-ray diffractogram of an amorphous solid dispersion of abiraterone and hydroxy propyl β cyclodextrin.

FIG. 4 is a graph reporting exemplary concentration of dissolved abiraterone versus time (dissolution profile) for neat crystalline abiraterone or various solid dispersions of abiraterone with a polymer excipient or a hydroxy propyl β cyclodextrin excipient.

FIG. 5 is a graph reporting exemplary concentration of dissolved abiraterone versus time (dissolution profile) for amorphous solid dispersions of abiraterone with a hydroxy propyl β cyclodextrin primary excipient in the presence of various polymer secondary excipients. Only the neutral phase dissolution profile is shown.

FIG. 6 is a set of exemplary X-ray diffractograms of amorphous solid dispersions of abiraterone with various amounts of a hydroxy propyl β cyclodextrin primary excipient, and various amounts of a hydroxy propyl methyl cellulose acetate succinate with 10-14% acetate substitution and 4-8% of succinate substitution as the secondary excipient.

FIG. 7A is a set of graphs reporting exemplary concentration of dissolved abiraterone versus time (dissolution profile), in the acidic phase, for amorphous solid dispersions of abiraterone with various amounts of a hydroxy propyl β cyclodextrin primary excipient, and various amounts of a hydroxy propyl methyl cellulose acetate succinate with 10-14% acetate substitution and 4-8% of succinate substitution as a secondary excipient.

FIG. 7B is a set of graphs reporting exemplary concentration of dissolved abiraterone versus time (dissolution profile), in the neutral phase, for amorphous solid dispersions of abiraterone with various amounts of a hydroxy propyl β cyclodextrin primary excipient, and various amounts of a hydroxy propyl methyl cellulose acetate succinate with 10-14% acetate substitution and 4-8% of succinate substitution as a secondary excipient.

FIG. 8 is a graph reporting exemplary concentration of dissolved abiraterone versus time (dissolution profile) as a function of the amount of drug loaded into the dissolution vessel (25 to 200 times the intrinsic solubility) for amorphous solid dispersions of abiraterone with a polymer excipient or a hydroxy propyl β cyclodextrin primary excipient and hydroxy propyl methyl cellulose acetate succinate with 10-14% acetate substitution and 4-8% of succinate substitution as a secondary excipient.

FIG. 9 is an exemplary X-ray diffractogram of amorphous solid dispersions of abiraterone and hydroxy propyl β cyclodextrin in 1:4 (Example 7.1) and 3:7 (Example 7.2) weight ratios formed by thermokinetic processing.

FIG. 10 is a graph reporting exemplary concentration of dissolved abiraterone versus time (dissolution profile) for solid dispersions of abiraterone and hydroxy propyl β cyclodextrin in weight ratios of 1:9 (Example 2.4), 1:4 (Example 7.1), and 3:7 (Example 7.2) formed by thermokinetic compounding.

FIG. 11 is an exemplary X-ray diffractogram of neat crystalline abiraterone acetate.

FIG. 12 is a set of exemplary X-ray diffractograms of abiraterone acetate solid dispersions with various polymer excipients. Excipient type (cellulose-based, polyvinyl-based, or acrylate-based) is indicated.

FIG. 13 is a graph reporting exemplary concentration of dissolved abiraterone acetate versus time (dissolution profile) for neat crystalline abiraterone acetate or various solid dispersions of abiraterone acetate with a polymer excipient.

FIG. 14 is an exemplary X-ray diffractogram of an amorphous solid dispersion of abiraterone acetate and hydroxy propyl β cyclodextrin.

FIG. 15 is a graph reporting exemplary concentration of dissolved abiraterone acetate versus time (dissolution profile) for neat crystalline abiraterone acetate and an amorphous solid dispersion of abiraterone acetate with hydroxy propyl β cyclodextrin.

FIG. 16 is an exemplary X-ray diffractogram of amorphous solid dispersions of abiraterone acetate and hydroxy propyl β cyclodextrin in 1:4 (Example 10.1) weight ratio formed by thermokinetic processing.

FIG. 17 is a graph reporting exemplary concentration of dissolved abiraterone acetate versus time (dissolution profile) for solid dispersions of abiraterone acetate and hydroxy propyl β cyclodextrin in weight ratios of 1:9 (Example 9.1) and 1:4 (Example 10.1)

FIG. 18 is a graph reporting exemplary concentration of dissolved abiraterone acetate versus time (dissolution profile) as a function of the amount of drug loaded into the dissolution vessel (100 to 400 times the intrinsic solubility) in the form of an abiraterone acetate-hydroxy propyl β cyclodextrin (1:9 w/w) ASD.

FIG. 19 is a graph reporting exemplary abiraterone concentration versus time from dissolution testing of 50 mg tablets made per Example 10 in 900 ml of 0.01 N HCl.

FIG. 20 is a graph reporting exemplary abiraterone plasma concentration versus time profiles following oral administration to male beagle dogs of abiraterone IR and XR tablets (50 mg abiraterone) made per Examples 11.1 and 11.2, respectively, relative to the reference, Zytiga (250 mg abiraterone acetate).

FIG. 21 is a graph reporting exemplary total oral abiraterone exposure (AUC) versus dose curve from an ascending dose PK study in SCID mice comparing the composition made per Example 2.4 versus abiraterone acetate.

FIG. 22 is a graph reporting exemplary tumor growth curves following once-daily administration of abiraterone acetate or the composition from Example 2.4 at two dose levels to 22RV1 xenograft mice.

FIG. 23 is a graph reporting exemplary geometric mean abiraterone plasma levels (ng/mL) in healthy male subjects following treatment with 200 mg DST-2970 IR, fasted state; 200 mg DST-2970 IR, fed state; or 1,000 mg ZYTIGA®, fasted state.

FIG. 24 is a graph reporting exemplary arithmetic mean abiraterone plasma levels (ng/mL) in healthy male subjects following treatment with 200 mg DST-2970 IR, fasted state; 200 mg DST-2970 IR, fed state; or 1,000 mg ZYTIGA®, fasted state.

FIG. 25 is a graph reporting exemplary individual patient data of abiraterone plasma levels (ng/mL) in healthy male subjects following treatment with 200 mg DST-2970 IR, fasted state.

FIG. 26 is a graph reporting exemplary individual patient data of abiraterone plasma levels (ng/mL) in healthy male subjects following treatment with 200 mg DST-2970 IR, fed state.

FIG. 27 is a graph reporting exemplary individual patient data of abiraterone plasma levels (ng/mL) in healthy male subjects following treatment with 1,000 mg ZYTIGA®, fasted state.

FIG. 28 is a set of example X-ray diffractograms of (upper Panel) neat abiraterone API, melt quenched abiraterone API and HPBCD; (lower Panel) Lot 1 PM and Lots 1 to 5 KinetiSol® Solid Dispersions KSDs) (DisperSol Technologies LLC, Texas). (The dotted circle on top figure indicates the characteristic peaks of abiraterone API and the dotted lines in the bottom figure indicates the peak position region of these characteristic peaks).

FIG. 29 is an example mDSC thermogram of Lot 1 PM and Lots 1 to 5 KSDs.

FIG. 30 is example ¹³C ssNMR spectra of neat abiraterone API, HPBCD, Lot 1 PM and Lots 1 to 5 KSDs. (The dotted rectangles indicate the regions of sp³ hybridized carbon atoms, C3 carbon atom and sp² hybridized carbon atoms of neat abiraterone API).

FIG. 31 is example 2D ¹³C-¹H HETCOR spectra of neat abiraterone API (black), HPBCD (red) and Lot 3 KSD (blue). ¹H cross sestions at 103.9 ppm in the ¹³C dimention are shown in the 2D spectra.

FIG. 32 is a graph reporting example phase solubility profiles for abiraterone-HPBCD in 0.01N HCl and FaSSIF.

FIG. 33 is a graph reporting X-ray diffractograms of Lots 1 to 3 KSDs at 90° C. and 150° C. (The dotted lines indicate the peak position region of abiraterone characteristic peaks).

FIG. 34 is a graph reporting example in vitro, non-sink, gastric transfer dissolution profiles of neat abiraterone API and Lots 1 to 5 KSDs; red region (0.01N HCl) and blue region (FaSSIF).

FIG. 35 is a graph reporting example in vivo average plasma concentration v/s time profiles from oral dosing of Zytiga® and Lots 1 to 3 Tablets in fasted non-naïve male beagle dogs.

FIG. 36 is a graph reporting example in vitro and in vivo percent relative performance of KSDs with various drug loadings.

FIG. 37 is a schematic showing examples of cyclodextrin host molecule and API guest molecule of inclusion complexes having one host molecule and one guest molecule (left Panel), or two host molecules and one guest molecule (right Panel).

FIG. 38 shows the molecular structure of β cyclodextrin, in which R═CH₂CHOHCH₃ or H, having varying degrees of substitution at the 2, 3, and 6 positions.

### DETAILED DESCRIPTION

The present disclosure relates to abiraterone pharmaceutical formulations and methods of forming and administering such pharmaceutical formulations.

### A. Pharmaceutical Formulation

A pharmaceutical formulation of the present disclosure may include abiraterone as an active pharmaceutical ingredient (API). Abiraterone, unless otherwise specified herein, includes both the active form of abiraterone and its modified forms, in either amorphous or crystalline states. Modified forms of abiraterone include a pharmaceutically acceptable salt, ester, derivative, analog, prodrug, hydrate, or solvate thereof. In certain embodiments, the term abiraterone excludes a prodrug of abiraterone, such as abiraterone acetate.

Abiraterone is (3β)-17-(3-pyridinyl)androsta-5,16-dien-3-ol and has the formula:

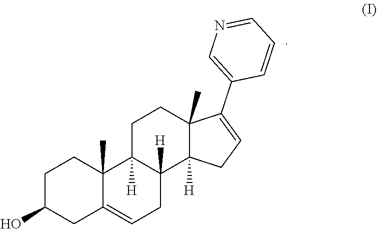

Abiraterone acetate, such as ZYTIGA®, is an ester of abiraterone, (3β)-17-(3-pyridinyl)androsta-5, 16-dien-3-ol acetate, and has the formula:

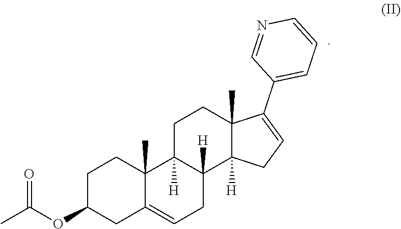

The pharmaceutical formulation may include abiraterone, which, prior to the present disclosure, has proven resistant to pharmaceutical formulation with sufficient bioavailability or therapeutic effect. In particular, to the extent a pharmaceutical formulation of the present disclosure includes both abiraterone and in a modified form, such as a pharmaceutically acceptable salt, ester, derivative, analog, prodrug, hydrate, or solvate thereof, the pharmaceutical formulation may include at least 80%, at least 95%, at least 99%, or at least 99% abiraterone as compared to total abiraterone and modified abiraterone by molecular percentage, by weight, or by volume.

The abiraterone in a pharmaceutical formulation of the present disclosure may lack substantial impurities. For example, the abiraterone may lack impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July 2006, incorporated by reference herein. Alternatively, the abiraterone in a pharmaceutical formulation of the present disclosure may have less than 1.0%, 0.75%, 0.5%, 0.1%, 0.05%, or 0.01% impurities by weight as compared to total weight of abiraterone and impurities, relative to a standard of known concentration in mg/mL. As another alternative, the abiraterone in a pharmaceutical formulation of the present disclosure may retain at least 90%, 91%, 92%, 93%, 94%, 95%, 96%, 97%, 98%, 99%, or 99.9% drug activity or potency as compared to the uncompounded abiraterone as measured by HPLC. Impurities may include abiraterone degradation products, such as thermal degradation products. In specific examples, a pharmaceutical formulation of the present disclosure including abiraterone may further include a glucocorticoid replacement API. Suitable glucocorticoid replacement APIs may have an intermediate biological half-life, such as between 18 and 36 hours, or a long biological half-life, such as between 36 and 54 hours. Suitable glucocorticoid APIs include dexamethasone, prednisone or prednisolone or alkylated forms, such as methyl prednisone and methyl prednisolone, and any combinations thereof. Other glucocorticoid replacement APIs may also be used.

The glucocorticoid replacement API in a pharmaceutical formulation of the present disclosure may also not contain substantial levels of impurities. For example, the glucocorticoid replacement may not have impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the glucocorticoid replacement API in a pharmaceutical formulation of the present disclosure may be have less than 1.0%, 0.75%, 0.5%, 0.1%, 0.05%, or 0.01% impurities by weight as compared to total weight of glucocorticoid replacement API and impurities, relative to a standard of known concentration in mg/mL. As another alternative, the glucocorticoid replacement API in a pharmaceutical formulation of the present disclosure may retain at least 90%, 91%, 92%, 93%, 94%, 95%, 96%, 97%, 98%, 99%, or 99.9% drug activity or potency as compared to the uncompounded glucocorticoid replacement API as measured by HPLC. Impurities may include glucocorticoid replacement API degradation products, such as thermal degradation products.

A pharmaceutical formulation of the present disclosure may further include one or more other APIs in addition to abiraterone. Suitable additional APIs include other APIs approved to treat prostate cancer, or a side effect of prostate cancer or prostate cancer treatment. These additional APIs may be in their active form. These APIs may be compoundable even when they have not been previously compoundable, compoundable in an orally administrable pharmaceutical formulation, compoundable with abiraterone, or compoundable in their active forms. Suitable additional APIs include those used in androgen-deprivation therapy, non-steroidal androgen receptor inhibitors, taxanes, gonadotrophin-releasing hormone antagonists, gonadotropin-releasing hormone analogs, androgen receptor antagonists, non-steroidal anti-androgens, analogs of luteinizing hormone-releasing hormone, anthracenedione antibiotics, and radiopharmaceuticals, and any combinations thereof. These suitable additional APIs include apalutamide, such as ERLEADA™ (Janssen), bicalutamide, such as CASODEX® (AstraZeneca, North Carolina, US), cabazitaxel, such as JEVTANA® (Sanofi-Aventis, France), degarelix, docetaxel, such as TAXOTERE® (Sanofi-Aventis), enzalutamide, such as XTANDI® (Astellas Pharma, Japan), flutamide, goserelin acetate, such as ZOLADEX® (TerSera Therapeutics, Iowa, US), leuprolide acetate, such as LUPRON® (Abbvie, Ill., US), LUPRON® DEPOT (Abbvie), LUPRON® DEPOT-PED (Abbvie), and VIADUR® (ALZA Corporation, California, US), mitoxantrone hydrochloride, nilutamide, such as NILANDRON® (Concordia Pharmaceuticals, Barbados), and radium 223 dichloride, such a XOFIGO® (Bayer Healthcare Pharmaceuticals, N.J., US), and any combinations thereof.

Any additional API in a pharmaceutical formulation of the present disclosure may also not contain substantial levels of impurities. For example, the additional API may not have impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the additional API in a pharmaceutical formulation of the present disclosure may be have less than 1.0%, 0.75%, 0.5%, 0.1%, 0.05%, or 0.01% impurities by weight as compared to total weight of additional API and impurities, relative to a standard of known concentration in mg/mL. As another alternative, the additional API in a pharmaceutical formulation of the present disclosure may retain at least 90%, 91%, 92%, 93%, 94%, 95%, 96%, 97%, 98%, 99%, or 99.9% drug activity or potency as compared to the uncompounded additional API as measured by HPLC. Impurities may include additional API degradation products, such as thermal degradation products.

A pharmaceutical formulation of the present disclosure further includes at least one excipient. When multiple excipients are used in a pharmaceutical formulation of the present disclosure, the one present in the largest amount by weight percent is typically referred to as the primary excipient, with other excipients being designated the secondary excipient, tertiary excipient and so forth based on descending amounts by weight percent.

A pharmaceutical formulation of the present disclosure may further include a cyclic oligomer excipient, such as a cyclic oligosaccharide or cyclic oligosaccharide derivative excipient, a cyclic peptide oligomer or cyclic peptide oligomer derivative, or a cyclic polycarbonate oligomer or cyclic polycarbonate oligomer derivative, and any combinations thereof. An oligosaccharide excipient may have between 3 to 15 saccharide monomer units, such as glucose units and glucose derivative units, fructose units and fructose derivative units, galactose and galactose derivative units, and any combinations thereof. The saccharide monomer units may be derivatized with a functional group, for example a sulfobutylether, or a hydroxypropyl derivative, or a carboxymethyl derivative or by methylation.

For example, the pharmaceutical formulation of the present disclosure may include a cyclodextrin (CD).

Cyclodextrins (CD) are cyclic oligomers containing at least six D-(+)-glucopyranose units attached by α(1→4) glycosidic bonds (Davis, Mark E., and Marcus E. Brewster. 2004. ‘Cyclodextrin-based pharmaceutics: past, present and future’, Nature Reviews Drug Discovery, 3: 1023-35; Loftsson, T., P. Jarho, M. Masson, and T. Jarvinen. 2005. ‘Cyclodextrins in drug delivery’, Expert Opin Drug Deliv, 2: 335-51.). The glucopyranose units in CDs, are present in chair conformation, thereby giving CDs a truncated cone like or toroidal structure (Loftsson, T., P. Jarho, M. Masson, and T. Jarvinen. 2005. ‘Cyclodextrins in drug delivery’, Expert Opin Drug Deliv, 2: 335-51.). The outer surface of CDs have secondary hydroxy groups extending from the wider edge and the primary groups from the narrow edge of the cone, which imparts hydrophilic nature to the outer surface (Sharma, Neha, and Ashish Baldi. 2016. ‘Exploring versatile applications of cyclodextrins: an overview’, Drug Delivery, 23: 729-47.). The inner cavity of CDs contain skeletal carbons with hydrogen atoms and oxygen bridges, which imparts lipophilic nature to the inner cavity (Sharma, Neha, and Ashish Baldi. 2016. ‘Exploring versatile applications of cyclodextrins: an overview’, Drug Delivery, 23: 729-47.).

Accordingly, in some embodiments, the present disclosure provides pharmaceutical formulations including an inclusion complex of abiraterone with a cyclic oligomer, such as a cyclodextrin, or other cyclic oligomers described herein, and methods of forming such pharmaceutical formulations. The inclusion complexes in the pharmaceutical formulations of the present disclosure differ from amorphous solid dispersions in which the abiraterone is dispersed between excipient molecules. The inclusion complexes in the pharmaceutical formulations of the present disclosure include amorphous abiraterone that is included within the structure of the cyclic oligomer molecule. In particular, in some embodiments, the inclusion complexes in the pharmaceutical formulations of the present disclosure may be formed using a process that includes thermokinetic compounding of abiraterone and a cyclic oligomer. As described herein, in some embodiments, the properties of the inclusion complex of the abiraterone and the cyclic oligomer is dependent upon the molar ratio of the abiraterone to the cyclic oligomer, and the properties of the abiraterone and the cyclic oligomer. The inclusion complexes in pharmaceutical formulations of the present disclosure may provide increased solubility, bioavailability, or both of the abiraterone, and correspondingly improved drug properties for administration to patients to treat conditions responsive to abiraterone. In addition, formation of inclusion complexes including abiraterone, using a process of thermokinetic compounding as described herein, without use of solvent, external heat and only with high shear mixing, in solid state, is surprising and unexpected.

For example, in some embodiments, cyclic oligomers such as CDs, or other cyclic oligomers described herein, can form an inclusion complex with an abiraterone, or a portion of an abiraterone, by including abiraterone or a portion thereof into the lipophilic central cavity of the cyclic oligomer through non-covalent interactions. By forming partial or complete inclusion complexes with abiraterone, a CD or other cyclic oligomer can impart higher aqueous solubility to the abiraterone and increase bioavailability of the abiraterone.

The cyclodextrins (CDs) of the present disclosure include α-CD, β-CD and γ-CD containing six, seven and eight glucopyranose units respectively (Loftsson, T., P. Jarho, M. Masson, and T. Jarvinen. 2005. ‘Cyclodextrins in drug delivery’, Expert Opin Drug Deliv, 2: 335-51.), and derivatives thereof.

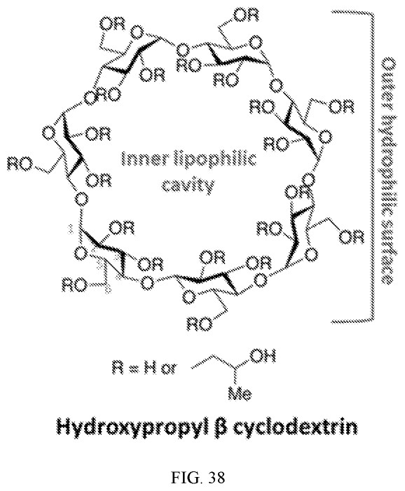

The CDs of the present disclosure may include, substitutions of one or more hydroxy groups. Without limitation to theory, substitution of hydroxy groups of CDs with hydrophobic or hydrophilic groups, may be used to impart higher aqueous solubility to the CDs by interrupting their intermolecular hydrogen bonding. For example, hydroxypropyl substitution on β-CD, to form hydroxy propyl β cyclodextrin (HPBCD) (FIG. 38), increases its water solubility from 18.5 mg/ml to >600 mg/ml respectively (Loftsson, T., P. Jarho, M. Masson, and T. Jarvinen. 2005. ‘Cyclodextrins in drug delivery’, Expert Opin Drug Deliv, 2: 335-51.). Accordingly, in some embodiments, the β-CD may include one or more R-groups for example as shown in FIG. 38, wherein R═CH₂CHOHCH₃ or H, having varying degrees of substitution at the 2, 3, and 6 positions).

For example, the pharmaceutical formulation may include a cyclodextrin, such as a cyclodextrin containing 6, 7 or 8 monomer units, in particular an β cyclodextrin, such as CAVAMAX® W6 Pharma (Wacker Chemie AG, Germany), a β cyclodextrin, such as CAVAMAX® W7 Pharma (Wacker Chemie), or a γ cyclodextrin, such as CAVAMAX® W8 Pharma (Wacker Chemie). Cyclodextrins contain dextrose units of (α-1,4)-linked α-D-glucopyranose that form acyclic structure having a lipophilic central cavity and a hydrophilic outer surface. Suitable cyclodextrins also include hydroxypropyl β cyclodextrin, such as KLEPTOSE® HBP (Roquette, France) and Na sulfo-butyl ether β cyclodextrin, such as DEXOLVE® 7 (Cyclolab, Ltd., Hungary).

Derivatization may facilitate the use of cyclic oligomer excipients in thermokinetic compounding.

Particularly when used in a thermokinetic compounding process, particle size of a cyclic oligomer excipient may facilitate compounding. Derivatization, pre-treatment, such as by slugging or granulation, or both, may increase or decrease particle size of a cyclic oligomer excipient to be within an optimal range. For example, the average particle size of a cyclic oligomer excipient may be increased by up to 500%, or up to 1,000%, by between 50% and 500%, or by between 50% and 1,000%. The average particle size of a cyclic oligomer excipient may be decreased by up to 50%, or up to 90%, or by between 5% and 50% or by between 5% and 90%.

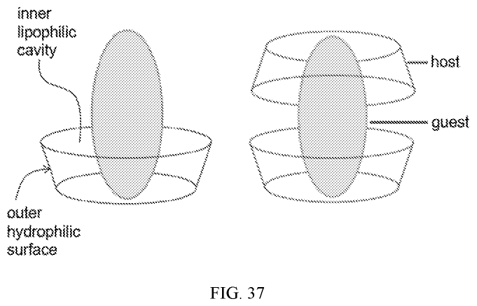

The inclusion complexes of the present disclosure may be referred to as a host-guest inclusion complex, in which a cyclic oligomer is the host and the abiraterone is the guest. For example, FIG. 37 shows in schematic form, non-limiting examples of host-guest inclusion complexes. In one non-limiting example, FIG. 37, left Panel, schematically depicts one unit of a host molecule, such as a cyclic oligomer, e.g., a cyclodextrin such as hydroxypropyl β cyclodextrin (HPBCD), in which a guest molecule, the abiraterone, is included, or at least a portion of the abiraterone molecule is included. In another non-limiting example, FIG. 37, right Panel, schematically depicts two units of a host molecule, such as a cyclic oligomer, e.g., a cyclodextrin such as hydroxypropyl β cyclodextrin (HPBCD), in which a guest molecule, the abiraterone, is included, or at least a portion of the abiraterone molecule is included.

A pharmaceutical formulation of the present disclosure may be prepared using thermokinetic compounding, as described herein. Without being limited by theory, thermokinetic compounding of the abiraterone together with the cyclic oligomer may provide an advantage in formulating the pharmaceutical formulations of the present disclosure such that the thermokinetic compounding process allows more intimate mixing of the abiraterone with the cyclic oligomer than is possible using some other methods of formulation. In particular, for example, as discussed above, cyclodextrins have truncated cone like or toroidal structure, with the larger and the smaller openings of the toroid exposing to the solvent secondary and primary hydroxyl groups respectively. Without limitation to theory, in some implementations, thermokinetic compounding may allow the abiraterone to become included by the truncated cone like or toroidal structure of the cyclodextrin. The thermokinetic compounding may provide increased efficiency and/or percentage of inclusion of the available abiraterone within the cyclodextrin structure compared to other formulation methods. Accordingly, the inclusion of the abiraterone within the cyclodextrin may provide improved solubility and bioavailability of the abiraterone.

Accordingly, in some embodiments, the present disclosure relates to methods of forming a pharmaceutical formulation of the abiraterone and a cyclic oligomer having an optimal drug loading of the abiraterone in an inclusion complex with the cyclic oligomer.

The term “drug loading” generally refers to the amount of an API incorporated into the pharmaceutical formulation. In particular, the term “drug loading” as used herein refers to the amount of abiraterone that can be included in an inclusion complex within a cyclic oligomer in the pharmaceutical formulation. In some embodiments, a method of forming a pharmaceutical formulation of the present disclosure that includes a thermokinetic compounding process provides increased drug loading as compared to other methods of formulating pharmaceutical formulations, meaning an increased amount of abiraterone that can be included in an inclusion complex within a cyclic oligomer in the pharmaceutical formulation, and a corresponding decrease in the amount of unincluded abiraterone that may be present as an amorphous dispersion between the cyclic oligomers of the pharmaceutical formulation.

For example, in general for solid dispersions, when the drug loading is less than the equilibrium solubility of the crystalline drug in the polymer carrier, the system is thermodynamically stable and the drug is molecularly dispersed in the polymer carrier matrix, forming a homogenous system (Huang and Dai 2014). Such a system may not be practical since for some drug-polymer carrier systems, as this would occur at extremely low drug loadings (Huang and Dai 2014). When the drug loading is high in solid dispersions, such as when it is higher than the equilibrium solubility of the amorphous drug in a polymer carrier, such systems are highly unstable and can lead to spontaneous phase separation and crystallization, thereby negatively affecting the stability and performance of solid dispersions (Qian, Huang, and Hussain 2010). Moreover, when cyclic oligomers such as CDs are excipients for pharmaceutical formulations, it cannot not be assumed that low drug loading is necessarily beneficial. This is because low drug loading would mean a higher amount of CD, which can hamper drug absorption from the gastrointestinal tract and lead to lower bioavailability (Loftsson et al. 2016; Loftsson and Brewster 2012). Also problems associated with high drug loading as discussed above are also true for pharmaceutical formulations including an API and a cyclic oligomer, for example when an API is present in higher than the equilibrium solubility of the amorphous drug in a cyclic oligomer carrier, such systems may be highly unstable and can lead to spontaneous phase separation and crystallization, thereby negatively affecting the stability and performance of the pharmaceutical formulation. Thus, identification of optimal drug loading is important when the carrier is a cyclic oligomer, for example such as HPBCD.

In some embodiments, an inclusion complex in a pharmaceutical formulation of the present disclosure may have drug loading of up to 10%, 20%, 30%, 40%, 50%, 60%, 70%, 80%, or 90% weight of the abiraterone as a total weight of the pharmaceutical formulation including the weight of the abiraterone plus the weight of the cyclic oligomer.

For example, in some embodiments, an inclusion complex of a pharmaceutical formulation of the present disclosure may have drug loading of up to 10%, 20%, or 30% weight of the abiraterone as a total weight of the pharmaceutical formulation including the weight of the abiraterone plus the weight of the cyclic oligomer, such as HPBCD, wherein the abiraterone is in substantially physically amorphous and chemically stable form (e.g., see Examples 24 and 27).

As would be understood by skilled persons, for a given combination of abiraterone and a cyclic oligomer, the relationship between percentage weight of drug loading and molar ratio of abiraterone to cyclic oligomer may be calculated by taking into account the molecular weight (e.g., in g/mol) of each of the abiraterone and the cyclic oligomer.

In some embodiments described herein, the inclusion complex may have a molar ratio of guest molecule : host molecule from 1:0.25 to 1:25, for example such as, or such as about, 2:1, 1:1, or 1:2. Accordingly, in some embodiments of the pharmaceutical formulation of the present disclosure, the abiraterone and the cyclic oligomer may be present in the pharmaceutical formulation in a molar ratio of abiraterone: cyclic oligomer from 1:0.25 to 1:25, for example such as, or such as about, 2:1, 1:1, or 1:2. For example, in some embodiments of the pharmaceutical formulation of the present disclosure, the abiraterone and the HPBCD may be present in the pharmaceutical formulation in a molar ratio of abiraterone:HPBCD from 1.0:2.2, 1.0:1.0, 1.0:0.6, 1.0:0.4 and 1.0:0.2. In some embodiments, the abiraterone and the HPBCD may be present in the pharmaceutical formulation in a molar ratio of abiraterone: HPBCD from 1.0:2.2 to1.0:0.6, such as, or such as about, 1:2, wherein the pharmaceutical formulation contain amorphous abiraterone which is at least partially complexed within HPBCD (e.g., see Example 25). Without limitation to theory, in some embodiments, a molar ratio of abiraterone: HPBCD of, or of about, 1:2 may allow complete inclusion of one molecule of abiraterone within two molecules of HPBCD. Similarly, without limitation to theory, in some embodiments, a drug loading of, or of about 10% abiraterone : 90% HPBCD may allow complete inclusion of one molecule of abiraterone within two molecules of HPBCD.

In some embodiments, a method of formulation that includes a thermokinetic compounding process provides increased inclusion complexation efficiency and correspondingly increased stability of amorphous abiraterone in the pharmaceutical formulation as compared to a pharmaceutical formulation having a same molar ratio of an abiraterone: cyclic oligomer formed using a method that does not include a thermokinetic compounding process. Without limitation to theory, thermokinetic compounding may allow increased stable inclusion complexation of the abiraterone at least partially within the interior of a cyclic oligomer, whereas other methods of formulation that do not include a thermokinetic compounding process may have an increased amount of unincluded abiraterone present outside the cyclic oligomer.

Accordingly, in some embodiments, the present disclosure provides a pharmaceutical formulation including an abiraterone, or a pharmaceutically acceptable salt, ester, derivative, analog, prodrug, hydrate, or solvate thereof; and a cyclic oligomer excipient; wherein at least 90%, 91%, 92%, 93%, 94%, 95%, 96%, 97%, 98%, 99%, or 99.9% of the abiraterone is present in an inclusion complex within the cyclic oligomer.

In some embodiments, a pharmaceutical formulation of the present disclosure formed using a method that includes a thermokinetic compounding process may provide an increase of up to 10%, 20%, 30%, 40%, 50%, 60%, 70%, 80%, or 90% more inclusion of abiraterone within a cyclic oligomer as compared to a method that does not include a thermokinetic compounding process, or up to 2-fold, 3-fold, 4-fold, 5-fold, 6-fold, 7-fold, 8-fold, 9-fold, or 10-fold increase in inclusion of abiraterone within a cyclic oligomer as compared to a method that does not include a thermokinetic compounding process.

In some embodiments, the inclusion complexes of the pharmaceutical formulations of the present disclosure may have increased stability, both in terms of physical stability and chemical stability. The term “stability” as used herein includes physical stability, such as the abiraterone being maintained in an amorphous state in the pharmaceutical formulation, without crystallization or recrystallization of the abiraterone. The term “stability” as used herein also includes chemical stability, such as reduces incidence of API degradation, for example due to incompatibility with other excipients, heat exposure and light exposure. For example, the inclusion complexes of the present disclosure may have reduced crystallization of the abiraterone over time as compared to an amount of crystallization over time in a formulation including abiraterone in an amorphous solid dispersion that does not include abiraterone in an inclusion complex.

For example, stability of abiraterone in a pharmaceutical formulation of the present disclosure can be assessed using methods known in the art and identifiable by skilled persons upon reading the present disclosure, including but not limited to methods described in Examples 22-28, such as heating studies described herein, wherein a pharmaceutical formulation is heated at a selected temperature for a selected period of time, allowed to cool, and analyzed by X-ray diffraction (XRD).

Without limitation to theory, when each molecule of abiraterone is complexed with at least one molecule of a cyclic oligomer, each abiraterone may be thermally and kinetically stabilized by inclusion of at least a portion of the abiraterone molecule within at least one cyclic oligomer, such as shown schematically in FIG. 37, left Panel. Increasing the ratio of cyclic oligomer to abiraterone in some embodiments allows inclusion of the abiraterone within one or more, such as two, cyclic oligomers, as shown for example in FIG. 37, right Panel. Recrystallization of abiraterone that is unincluded within the cyclic oligomer can be detected as crystalline abiraterone by XRD analysis. For example, without limitation to theory, abiraterone in a pharmaceutical formulation of an abiraterone and a cyclic oligomer that may become recrystallized upon heating and subsequent cooling, and detected as crystalline form of the abiraterone using, e.g., XRD analysis, may be abiraterone that is not included within the cyclic oligomer, but rather may be present as amorphous abiraterone dispersed between the cyclic oligomers in the pharmaceutical formulation.

Accordingly, in some embodiments of the pharmaceutical formulation of the present disclosure, in response to heating the pharmaceutical formulation to a temperature up to 90% of the melting point of the crystalline form of the abiraterone and allowing the pharmaceutical formulation to cool to room temperature, less than 10%, 9%, 8%, 7%, 6%, 5%, 4%, 3%, 2%, 1%, or 0.1% of the abiraterone may be in crystalline form.

In some embodiments, a pharmaceutical formulation of the present disclosure formed using a method that includes a thermokinetic compounding process may provide an increase in stability of an amorphous abiraterone of up to 10%, 20%, 30%, 40%, 50%, 60%, 70%, 80%, 90% or more, as compared to a method that does not include a thermokinetic compounding process, or up to 2-fold, 3-fold, 4-fold, 5-fold, 6-fold, 7-fold, 8-fold, 9-fold, or 10-fold increase in stability of an amorphous abiraterone as compared to a method that does not include a thermokinetic compounding process.

Stability analysis can include analysis of pharmaceutical formulations of the present disclosure formed using one or more molar ratios of abiraterone: cyclic oligomer, in order to identify a molar ratio of abiraterone: cyclic oligomer that provides the highest drug loading of the abiraterone in the pharmaceutical formulation that has an acceptable level of stability.

In some embodiments, an acceptable level of stability may be substantially complete amorphicity, having substantially no re-crystallization of the abiraterone after heating, or, for example, less than 10%, 9%, 8%, 7%, 6%, 5%, 4%, 3%, 2%, 1%, or 0.1% re-crystallization of abiraterone after heating, as determined by X-ray diffraction.

For example, in some embodiments, pharmaceutical formulations of the present disclosure may remain substantially amorphous, having substantially no re-crystallization of abiraterone in response to heating to a temperature up to 150° C., for example as assessed by X-ray diffraction. For example, a pharmaceutical formulation having an inclusion complex including up to 20% abiraterone may have substantially no re-crystallization of abiraterone in response to heating to a temperature up to 150° C., for example as assessed by X-ray diffraction (e.g., see Example 27).

Other methods that can be used to assess stability of the abiraterone in the pharmaceutical formulations of the present disclosure include, modulated differential scanning calorimetry (mDSC), Raman spectroscopy, and solid state Nuclear Magnetic Resonance Spectroscopy (ssNMR) (e.g., see Examples 22-28).

In general, the amorphous nature or the extent of inclusion of the abiraterone within the cyclic oligomer of the inclusion complex may be analyzed using X-ray diffraction (XRD), which may not exhibit strong peaks characteristic of a largely crystalline material. The amorphous nature or the extent of inclusion of the abiraterone within the cyclic oligomer of the inclusion complex may also be analyzed using other methods described herein, such as modulated differential scanning calorimetry (mDSC), solid state Nuclear Magnetic Resonance Spectroscopy (ssNMR), Raman spectroscopy, phase solubility analysis, stability analysis, in vitro dissolution studies. Examples of these methods are described in Examples 22-28 of the present disclosure. For example, the extent of abiraterone-cyclic oligomer inclusion complex formation in the pharmaceutical formulation may be evidenced by decrease in intensity of drug melting endotherm in differential scanning calorimetry, or by the magnitude of peak shifts in Raman spectroscopy, or by peak broadening in nuclear magnetic resonance spectroscopy.

A pharmaceutical formulation of the present disclosure may also include one or more additional excipients. These additional excipients may particularly include a polymer excipient or combination of polymer excipients. Suitable polymer excipients include may be water-soluble. Suitable polymer excipients may also be ionic or non-ionic.

Suitable polymer excipients include a cellulose-based polymer, a polyvinyl-based polymer, or an acrylate-based polymer. These polymers may have varying degrees of polymerization or functional groups.

Suitable cellulose-based polymers include an alkylcellulose, such as a methyl cellulose, a hydroxyalkylcellulose, or a hydroxyalkyl alkylcellulose. Suitable cellulose-based polymers more particularly include hydroxymethylcellulose, hydroxyethyl methylcellulose, hydroxyethylcellulose, hydroxypropylcellulose, hydroxybutylcellulose, hydroxypropylcellulose, hydroxypropylmethylcellulose, such as METHOCEL™ E3 and METHOCEL™ E5 (Dow Chemical, Michigan, US); ethylcellulose, such as ETHOCEL® (Dow Chemical), cellulose acetate butyrate, hydroxyethylcellulose, sodium carboxymethyl-cellulose, hydroxypropylmethylcellulose acetate succinate, such as AFFINISOL® HPMCAS 126 G (Dow Chemical), cellulose acetate, cellulose acetate phthalate, such as AQUATERIC™ (FMC, Pennsylvania, US), carboxymethylcellulose, such as sodium carboxymethycellulose, hydroxyethyl methyl cellulose, hydroxypropyl cellulose, hydroxyethyl cellulose, hydroxymethyl cellulose, crystalline cellulose, and any combinations thereof.

Suitable polyvinyl-based polymers include polyvinyl alcohol, such as polyvinyl alcohol 4-88, such as EMPROVE® (Millipore Sigma, Massachusetts, US) polyvinyl pyrrolidone, such as LUVITEK® (BASF, Germany) and KOLLIDON® 30 (BASF), polyvinylpyrrolidone-co-vinylacetate, poly(vinyl acetate)-co-poly(vinylpyrrolidone) copolymer, such as KOLLIDON® SR (BASF), poly(vinyl acetate) phthalate, such as COATERIC® (Berwind Pharmaceutical Services, Pennsylvania, US) or PHTHALAVIN® (Berwind Pharmaceutical Services), polyvinyl caprolactam-polyvinyl acetate-polyethylene glycol graft copolymer, such as SOLUPLUS® (BASF), polyvinyl caprolactam-polyvinyl acetate-polyethylene glycol graft copolymer, such as SOLUPLUS® (BASF), hard polyvinylchloride, and any combinations thereof.

Suitable acrylate-based polymers include acrylate and methacrylate copolymer, type A copolymer of ethylacrylate, methyl methacrylate and a methacrylic acid ester with quaternary ammonium groups in a ratio of 1:2:0.1, such as EUDRAGIT® RS PO (Evonik, Germany), poly(meth)acrylate with a carboxylic acid functional group, such as EUDRAGIT® S100 (Evonik), dimethylaminoethyl methacrylate-methacrylic acid ester copolymer, ethylacrylate-methylmethacrylate copolymer, poly(methacrylate ethylacrylate) (1:1) copolymer, poly(methacrylate methylmethacrylate) (1:1) copolymer, poly(methacrylate methylmethacrylate) (1:2) copolymer, poly(methacrylic acid-co-ethyl acrylate) (1:1), such as EUDRAGIT® L-30-D (Evonik), poly(methacylic acid-co-ethyl acrylate) (1:1), such as EUDRAGIT® L100-55 (Evonik), poly(butyl methacylate-co-(2-dimethylaminoethyl) methacrylate-co-methyl methacrylate (1:2:1), such as EUDRAGIT® EPO (Evonik), methacrylic acid-ethacrylate copolymer, such as KOLLICOAT MAE 100-55 (BASF), polyacrylate, polymethacrylate, and any combinations thereof.

Certain polymer excipients are particularly well suited for use alone or in combinations as a secondary excipient with a cyclic oligomer primary excipient. The secondary polymer excipient may be water-soluble. The polymer secondary excipient may be ionic or non-ionic. Suitable secondary non-ionic polymer excipients include hydroxy propyl methyl cellulose, such as METHOCEL™ E15 (Dow Chemical, Michigan, US) or METHOCEL™ E50 (Dow Chemical), and polyvinylpyrrolidone, such as KOLLIDON® 90 (BASF, Germany) Suitable secondary ionic polymer excipients include hydroxy propyl methyl cellulose acetate succinate, such as AFFINISOL® HPMCAS 716 G (Dow Chemical), AFFINISOL® HPMCAS 912 G (Dow Chemical), and AFFINISOL® HPMCAS 126 G (Dow Chemical), polyvinyl acetate phthalate, such as PHTHALAVIN® (Berwind Pharmaceutical Services), methacrylic acid based copolymer, such as methacrylic acid-ethacrylate copolymer, such as EUDRAGIT® L100-55 (Evonik, Germany), and any combinations thereof.

One particularly well-suited secondary excipient includes hydroxy propyl methyl cellulose acetate succinate. The hydroxy propyl methyl cellulose acetate succinate may have 5-14%, more particularly 10-14%, and more particularly 12% acetate substitution. The hydroxy propyl methyl cellulose acetate succinate may have 4-18%, more particularly 4-8%, more particularly 6% succinate substitution.

A polymer excipient may include only one polymer, or a pharmaceutical formulation of the present disclosure may include a combination of polymer excipients.

Any excipient, including any cyclic oligomer excipient or any polymer excipient, in a pharmaceutical formulation of the present disclosure may also not contain substantial levels of impurities. For example, the excipient in a pharmaceutical formulation of the present disclosure may be have less than 1.0%, 0.75%, 0.5%, 0.1%, 0.05%, or 0.01% impurities by weight as compared to total weight of excipient and impurities, relative to a standard of known concentration in mg/mL. Impurities may include excipient degradation products, such as thermal degradation products.

A pharmaceutical formulation of the present disclosure may also include one or more lipids. A pharmaceutical formulation of the present disclosure may be formulated using one or more lipid technologies. Lipids may be synthetic, semi-synthetic, or natural lipids. Lipids may be anionic, cationic, or neutral. Exemplary lipids include fats, fatty acids such as saturated, monounsaturated, polyunsaturated, omega-3, alpha-linolenic acid (ALA), eicosapentaenoic (EPA) and docosahexaenoic acid (DHA), omega-6, arachidonic acid (AA), linoleic acid, conjugated linoleic acid (CLA), and trans fatty acids, short-chain fatty acids (SCFAs) such as alpha-lipoic acid, medium-chain fatty acids (MCFAs), long-chain fatty acids (LCFAs), very long-chain fatty acids (VLCFAs), monoglycerides, diglycerides, triglycerides, phospholipids such as lecithin (phosphatidylcholine), sterols, cholesterol, phytosterols (plant sterols and stanols), carotenoids such as astaxanthin, lutein and zeaxanthin, lycopene, vitamin A-related carotenoids, waxes, and any combinations thereof.

A pharmaceutical formulation of the present disclosure may be in the form of amorphous abiraterone and the excipient. The abiraterone may contain less than 5% crystalline abiraterone, less than 1% crystalline abiraterone, or no crystalline abiraterone. The amorphous nature of the pharmaceutical formulation may be confirmed using X-ray diffraction (XRD), which may not exhibit strong peaks characteristic of a largely crystalline material.

A pharmaceutical formulation of the present disclosure may be formed by any suitable method for making amorphous solid dispersions, such as thermokinetic compounding, hot-melt extrusion, or spray drying, among others described herein. Thermokinetic compounding may be particularly useful for excipients that experience degradation in hot melt extrusion or that do not have a common organic solvent system with abiraterone as to facilitate spray drying.

As discussed above, thermokinetic compounding may also be particularly useful in obtaining increased yields of inclusion of abiraterone within the cyclic oligomers of the inclusion complexes, as compared to other methods of formulation. Without limitation to theory, thermokinetic compounding may provide increased compounding forces, such as increased shear forces, facilitating greater inclusion complexation of the abiraterone within the lipophilic interior of the cyclic oligomer than is possible using other formulation methods. Accordingly, thermokinetic compounding may provide a decrease in the amount of abiraterone that remains unincluded in the cyclic oligomer in a inclusion complex of the present disclosure after formulation, as compared to the amount of abiraterone that remains unincluded in the cyclic oligomer after formulation using other formulation methods. In some embodiments, unincluded abiraterone may form amorphous abiraterone dispersed within the pharmaceutical formulation as an amorphous solid dispersion. In some embodiments, thermokinetic compounding may provide an increase of up to 5%, 10%, 15%, 20%, 25%, 30%, 35%, 40%, 45%, 50%, 55%, 60%, 65%, 70%, 75%, 80%, 85%, 90%, 95%, or 100% more inclusion complex formation of the abiraterone included within the cyclic oligomer than may be achieved using other formulation methods. In some embodiments, thermokinetic compounding may provide an increase of up to 2-fold, 3-fold, 4-fold, 5-fold, 6-fold, 7-fold, 8-fold, 9-fold, or 10- fold more inclusion complex formation of the abiraterone included within the cyclic oligomer than may be achieved using other formulation methods. Accordingly, thermokinetic compounding may facilitate formulation of abiraterone in inclusion complexes with cyclic oligomers, at higher concentrations, while maintaining solubility, bioavailability, or both, than is achievable using other formulation methods.

A pharmaceutical formulation of the present disclosure containing amorphous abiraterone may dissolve more readily in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeS SIF).

In some embodiments, a pharmaceutical formulation of the present disclosure containing up to 10% by weight amorphous abiraterone may have at least 13-17-fold, e.g., 15.7-fold, increased dissolution in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF). In some embodiments, a pharmaceutical formulation of the present disclosure containing up to 20% by weight amorphous abiraterone may have at least 10-14-fold, e.g., 12.1-fold, increased dissolution in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF). In some embodiments, a pharmaceutical formulation of the present disclosure containing up to 30% by weight amorphous abiraterone may have at least 5-9-fold, e.g., 7.2-fold, increased dissolution in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF). In some embodiments, a pharmaceutical formulation of the present disclosure containing up to 40% by weight amorphous abiraterone may have at least 3-7-fold, e.g., 5.0-fold, increased dissolution in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF). In some embodiments, a pharmaceutical formulation of the present disclosure containing up to 50% by weight amorphous abiraterone may have at least 2-5-fold, e.g., 3.1-fold, increased dissolution in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF) (see Example 28).

Alternatively, the pharmaceutical formulation may be incorporated into a final dosage form that modifies or extends the release of abiraterone. This may include an extended release, delayed release, and/or pulsatile release profiles and the like. The pharmaceutical formulation may be incorporated into a tablet dosage from including a hydrophilic matrix that forms a swollen hydrogel in the gastric environment. This formation of hydrogel is intended to (1) retain the tablet in the stomach and (2) retard the release of abiraterone so as to provide a continuous release of the drug over a period of about 24 hours. More specifically, the dosage form may be an extended release oral drug dosage form for releasing abiraterone into the stomach, duodenum and small intestine of a patient, and includes: a single or a plurality of solid particles consisting of abiraterone or a pharmaceutically acceptable salt or prodrug or hydrate or solvate thereof dispersed within a polymer or a combination of polymers that (i) swells unrestrained dimensionally by imbibing water from gastric fluid to increase the size of the particles to promote gastric retention in the stomach of the patient in which the fasted/fed mode has been induced; (ii) gradually the abiraterone diffuses or the polymer erodes over a time period of hours, where the diffusion or erosion commences upon contact with the gastric fluid; herein the abiraterone ASD is vital for solubilization of abiraterone upon diffusion or erosion; and (iii) releases abiraterone to the stomach, duodenum and small intestine of the patient, as a result of the diffusion or polymeric erosion at a rate corresponding to the time period. Exemplary polymers include polyethylene oxides, alkyl substituted cellulose materials and combinations thereof, for example, high molecular weight polyethylene oxides and high molecular weight or viscosity hydroxypropylmethyl cellulose materials. A particularly well-suited polymer combination includes combination of polyethylene oxide POLYOX™ WSR 301 and hydroxypropyl methyl cellulose Methocel® E4M, used at ˜24% w/w and ˜18% w/w of the final tablet dosage form, respectively. This dosage from is intended to produce a pharmacokinetic profile with a reduced Cₘₐₓ-to-Cₘᵢₙ ratio such that human plasma concentrations remain within the therapeutic window for the duration of treatment. This abiraterone pharmacokinetic profile is expected to provide more efficacious cancer treatment with similar or reduced side effects.

The example above is only one example by which one can achieve a prolonged release of the solubility enhanced abiraterone ASD and thereby minimizing the Cₘₐₓ-to-Cₘᵢₙ ratio in a patient. Another example is a pulsatile release dosage form containing a component designed to release the solubility enhanced abiraterone ASD immediately in the stomach and one or more additional components designed to release a pulse of abiraterone at different regions in the intestinal tract. This can be accomplished by applying a pH-sensitive coating to one or more abiraterone ASD-containing components whereby the coating is designed to dissolve and release the active in different regions along the GI tract depending upon environmental pH. These functionally coated components may also contain an acidifying agent to decrease the microenvironmental pH to promote solubility and dissolution of abiraterone.

Furthermore, there are a myriad of controlled release technologies that could be applied to generate an extended abiraterone release profile when starting from the solubility enhanced abiraterone ASD compositions disclosed herein. It is important to note that the abiraterone ASD composition is enabling to this approach as applying conventional controlled drug release technologies to crystalline abiraterone or abiraterone acetate would fail to provide adequate drug release along the GI tract owing to the poor solubility of these forms of the compound.

In a pharmaceutical formulation of the present disclosure, the cyclic oligomer may be the only excipient. The pharmaceutical formulation may include 1% to 50% by weight amorphous abiraterone, particularly abiraterone, and between 50% and 99% by weight of one or more cyclic oligomer excipients. Alternatively, the pharmaceutical formulation may include at least 5%, at least 10%, or at least 20% by weight amorphous abiraterone, particularly abiraterone. Also, alternatively, the pharmaceutical formulation may include at least 60% or at least 90% by weight of one or more cyclic oligomer excipients.

In another pharmaceutical formulation of the present disclosure, the cyclic oligomer may be the primary excipient. The pharmaceutical formulation may include 1% to 50% by weight amorphous abiraterone, particularly abiraterone, and between 50% and 99% by weight cyclic oligomer primary excipient. Alternatively, the pharmaceutical formulation may include at least 5%, at least 10%, or at least 20% by weight amorphous abiraterone, particularly abiraterone. Also alternatively, the pharmaceutical formulation may include at least 60% by weight cyclic oligomer excipient. The pharmaceutical formulation may further include at least 1% secondary excipient, particularly a polymer secondary excipient.

In another pharmaceutical formulation of the present disclosure, the cyclic oligomer may be the secondary excipient and the pharmaceutical formulation may further include a primary excipient, such as a polymer primary excipient. The pharmaceutical formulation may include 1% to 50% by weight amorphous abiraterone, particularly abiraterone, between 50% and 99% by weight primary excipient, and between 50% and 99% by weight cyclic oligomer secondary excipient. Alternatively, the pharmaceutical formulation may include at least 5%, at least 10%, or at least 20% by weight amorphous abiraterone, particularly abiraterone.

A pharmaceutical formulation of the present disclosure may include abiraterone and a cyclic oligomer excipient, particularly a hydroxy propyl β cyclodextrin excipient in a molar ratio of abiraterone to cyclic oligomer excipient of 1:0.25 to 1:25, such as at least 1:2.

In a particular example, a pharmaceutical formulation of the present disclosure may be an amorphous dispersion of 1% to 50%, particularly at least 10% by weight abiraterone form, 80% by weight hydroxy propyl β cyclodextrin primary excipient, and 1% to 49%, particularly at least 10% by weight hydroxy propyl methyl cellulose acetate succinate secondary excipient.

A pharmaceutical formulation of the present disclosure may include an amount of amorphous abiraterone sufficient to achieve the same or greater therapeutic effect, bioavailability, Cₘᵢₙ, Cₘₐₓ or Tₘₐₓ as a greater amount of crystalline abiraterone or crystalline abiraterone acetate, such as ZYTIGA®, when consumed on an empty stomach. A pharmaceutical formulation as described herein may substantially improve the solubility of abiraterone, which may facilitate the improvement in therapeutic effect, bioavailability, Cₘᵢₙ, Cₘₐₓ or Tₘₐₓ.

“Therapeutic effect” may be measured by a decrease in measurable PSA level in a patient over a course of treatment, such as a one-month course of treatment. Other scientifically accepted measures of therapeutic effect, such as those used in the course of obtaining regulatory approval, particularly FDA approval, may also be used to determine “therapeutic effect.”

“Bioavailability” is measured herein as the area under the drug plasma concentration versus time curve (AUC) from an administered unit dosage form. Absolute bioavailability is the bioavailability of an oral composition compared to an intravenous reference assumed to deliver 100% of the active into systemic circulation. The insolubility of abiraterone precludes intravenous delivery; therefore, the absolute bioavailability of abiraterone cannot be known. The absolute bioavailability of ZYTIGA® when administered as approved on an empty stomach must be less than 10% because its AUC increases 10-fold when administered with a high-fat meal. The increase in bioavailability of ZYTIGA® when administered with a high-fat meal is assumed to be the result of improved solubility of abiraterone acetate in the fed state. In order to facilitate comparisons, bioavailability in the present disclosure may be measured on an empty stomach, such as at least two hours after the last ingestion of food and at least one hour before the next ingestion of food.

For example, the relative bioavailability of abiraterone in a pharmaceutical formulation of the present disclosure as compared to ZYTIGA® or a comparable crystalline abiraterone acetate may be at least 500% greater or even at least 1,000% greater.

For example, as illustrated in Example 28, a pharmaceutical formulation of the present disclosure having up to 10% by weight abiraterone may provide up to 4-fold increase in bioavailability of abiraterone as a same or greater amount of compared to a greater amount of crystalline abiraterone or crystalline abiraterone acetate, such as ZYTIGA®, when consumed on an empty stomach. A pharmaceutical formulation of the present disclosure having up to 20% by weight abiraterone may provide up to 3-fold increase in bioavailability of abiraterone as a same or greater amount of compared to a greater amount of crystalline abiraterone or crystalline abiraterone acetate, such as ZYTIGA®, when consumed on an empty stomach. A pharmaceutical formulation of the present disclosure having up to 30% by weight abiraterone may provide up to 2-fold increase in bioavailability of abiraterone as a same or greater amount of compared to a greater amount of crystalline abiraterone or crystalline abiraterone acetate, such as ZYTIGA®, when consumed on an empty stomach (e.g., see Example 28).

As shown in Example 28, in some embodiments, a relative in vitro dissolution performance of pharmaceutical formulations including amorphous abiraterone decreased as the drug loading increased. Without limitation to theory, this may be attributed to decreased abiraterone-cyclic oligomer inclusion complexation, hence reduced abiraterone solubility enhancement with increased drug loading.

In particular, a pharmaceutical formulation of the present disclosure may include an amount of amorphous abiraterone sufficient to achieve the same therapeutic effect or the same bioavailability in a patient as 1000 mg of crystalline abiraterone acetate, such as ZYTIGA®, when consumed on an empty stomach, once daily. Such a pharmaceutical formulation may also include a glucocorticoid replacement API, such as 5 mg of glucocorticoid replacement API.

Alternatively, a pharmaceutical formulation of the present disclosure may include an amount of amorphous abiraterone sufficient to achieve the same therapeutic effect or the same bioavailability in a patient as 500 mg of crystalline abiraterone acetate, such as ZYTIGA®, when consumed on an empty stomach, twice daily. Such a pharmaceutical formulation may also include a glucocorticoid replacement API, such as 5 mg of glucocorticoid replacement API.

A pharmaceutical formulation of the present disclosure may be for oral administration and may be further processed, with or without further compounding, to facilitate oral administration.

A pharmaceutical formulation of the present disclosure may be further processed into a solid dosage form suitable for oral administration, such as a tablet or capsule.

In order to further increase therapeutic effect, bioavailability, Cₘᵢₙ, or Cₘₐₓ of the abiraterone, a pharmaceutical formulation of the present disclosure may be combined with an additional amount of the primary excipient, secondary (or tertiary, etc.) excipient, such as hydroxy propyl methyl cellulose acetate secondary excipient, or another suitable concentration enhancing polymer not part of the pharmaceutical formulation to produce the solid dosage form.

Concentration enhancing polymers suitable for use in the solid dosage form may include compositions that do not interact with abiraterone in an adverse manner. The concentration enhancing polymer may be neutral or ionizable. The concentration enhancing polymer may have an aqueous solubility of at least 0.1 mg/mL over at least a portion of or all of pH range 1-8; particularly at least a portion of or all of pH range 1-7 or at least a portion of or all of pH range 7-8. When the solid dosage form is dissolved in in 0.01 N HCl and biorelevant media, such as: Simulated Gastric Fluid (SGF), Fasted State Simulated Intestinal Fluid (FaSSIF), or Fed State Simulated Intestinal Fluid (FeSSIF), the concentration-enhancing polymer may increase the maximum abiraterone concentration dissolved in the biorelevant media by a factor of at least 1.25, at least 2, or at least 3 as compared to an identical solid dosage form lacking the concentration enhancing polymer. A similar increase in maximum abiraterone concentration in biorelevant media may be observed when additional primary or secondary (or tertiary, etc.) excipients not present in the pharmaceutical formulation are added to the dosage form.

### B. Methods of Formulating a Pharmaceutical Formulation

A pharmaceutical formulation of the present disclosure may be prepared using thermokinetic compounding, which is a method of compounding components until they are melt-blended. Thermokinetic compounding may be particularly useful for compounding heat-sensitive or thermolabile components. Thermokinetic compounding may provide brief processing times, low processing temperatures, high shear rates, and the ability to compound thermally incompatible materials.

Thermokinetic compounding may be carried out in a thermokinetic chamber using one or multiple speeds during a single, compounding operation on a batch of components to form a pharmaceutical formulation of the present disclosure.

A thermokinetic chamber includes a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During thermokinetic compounding, the shaft is rotated causing the components being compounded, such as particles of the components being compounded, to impinge upon the inside surface of the chamber and upon facial portions of the extensions. The shear of this impingement causes comminution, frictional heating, or both of the components and translates the rotational shaft energy into heating energy. Any heating energy generated during thermokinetic compounding is evolved from the mechanical energy input. Thermokinetic compounding is carried out without an external heat source. The thermokinetic chamber and components to be compounded are not pre-heated prior to commencement of thermokinetic compounding.

The thermokinetic chamber may include a temperature sensor to measure the temperature of the components or otherwise within the thermokinetic chamber.

During thermokinetic compounding, the average temperature of the thermokinetic chamber may increase to a pre-defined final temperature over the duration of the thermokinetic compounding to achieve thermokinetic compounding of the abiraterone and the excipient, and any other components of a pharmaceutical formulation of the present disclosure, such as an additional API, for example a glucocorticoid replacement API, an additional excipient, or both. The pre-defined final temperature may be such that degradation of the abiraterone, excipient, or other components is avoided or minimized Similarly, the one or multiple speeds of use during thermokinetic compounding may be such that thermal degradation of the abiraterone, excipient, or other components is avoided or minimized. As a result, the abiraterone, excipient, or other components of the solid amorphous dispersion may lack substantial impurities.

The average maximum temperature in the thermokinetic chamber during thermokinetic compounding may be less than the glass transition temperature, melting point, or molten transition point, of abiraterone or any other APIs present, one or all excipients, or one or all other components of the amorphous solid dispersion, or any combinations or sub-combinations of components.

Pressure, duration of thermokinetic compounding, and other environmental conditions such as pH, moisture, buffers, ionic strength of the components being mixed, and exposure to gasses, such as oxygen, may also be such that degradation of abiraterone or any other APIs present, one or all excipients, or one or all other components is avoided or minimized Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds.

Variations of thermokinetic compounding may be used depending on the amorphous solid dispersion and its components. For example, the thermokinetic chamber may be operated at a first speed to achieve a first process parameter, then operated at a second speed in the same thermokinetic compounding process to achieve a final process parameter. In other examples, the thermokinetic chamber may be operated at more than two speeds, or at only two speeds, but in more than two time internals, such as at a first speed, then at a second speed, then again at the first speed.

The abiraterone component may be in a crystalline or semi-crystalline form prior to thermokinetic compounding.

In another variation, abiraterone or other API particle size is reduced prior to thermokinetic compounding. This may be accomplished by milling, for example dry milling the crystalline form of the abiraterone or other API to a small particle size prior to thermokinetic compounding, wet milling the crystalline form of the abiraterone or other API with a pharmaceutically acceptable solvent to reduce the particle size prior to thermokinetic compounding, or melt milling the crystalline form of the abiraterone or other API with at least one excipient having limited miscibility with the crystalline form of the abiraterone or other API to reduce the particle size prior to thermokinetic compounding.

Another variation includes milling the crystalline form of the abiraterone or other API in the presence of an excipient to create an ordered mixture where the abiraterone or other API particles adhere to the surface of excipient particles, excipient particles adhere to the surface of API particles, or both.

The thermokinetically compounded amorphous solid dispersion may exhibit substantially complete amorphicity.

The pharmaceutical formulation of the present disclosure may be formulated without a solvent. For example, the pharmaceutical formulation of the present disclosure may be prepared using thermokinetic compounding without a solvent. Accordingly, a pharmaceutical formulation of the present disclosure prepared by thermokinetic compounding may have no solvent in the pharmaceutical formulation or a tablet thereof and may have no impurities including the solvent in the pharmaceutical formulation or a tablet thereof.

In certain embodiments, the pharmaceutical formulation of the present disclosure prepared by thermokinetic compounding may exclude a prodrug of abiraterone, such as abiraterone acetate.

In certain embodiments, the pharmaceutical formulation of the present disclosure prepared by thermokinetic compounding may include a prodrug of abiraterone, such as abiraterone acetate, wherein the pharmaceutical formulation may have no solvent in the pharmaceutical formulation or a tablet thereof, and may have no impurities including the solvent in the pharmaceutical formulation or a tablet thereof.

In some embodiments, the compounding in the thermokinetic mixer provides a pharmaceutical formulation including an inclusion complex of the abiraterone and the cyclic oligomer excipient having an increase of up to 10%, 20%, 30%, 40%, 50%, 60%, 70%, 80%, 90% or more inclusion of the abiraterone within the cyclic oligomer as compared to a method that does not include a thermokinetic compounding process.

In some embodiments, the compounding in the thermokinetic mixer provides a pharmaceutical formulation including an inclusion complex of the abiraterone and the cyclic oligomer excipient having an increase of up to 2-fold, 3-fold, 4-fold, 5-fold, 6-fold, 7-fold, 8-fold, 9-fold, or 10-fold increase in inclusion of abiraterone within a cyclic oligomer as compared to a method that does not include a thermokinetic compounding process.

A pharmaceutical formulation of the present disclosure may be prepared using hot melt extrusion, whereby an excipient blend is heated to a molten state and subsequently forced through an orifice where the extruded product is formed into its final shape in which it is solidified upon cooling. The blend is conveyed through various heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw flights by which the various components of the powder blend are well mixed and de-aggregated.

The hot-melt extrusion equipment is typically a single or twin-screw apparatus but can be composed of more than two screw elements. A typical hot-melt extrusion apparatus contains a mixing/conveying zone, a heating/melting zone, and a pumping zone in succession up to the orifice. In the mixing/conveying zone, the powder blends are mixed and aggregates are reduced to primary particles by the shear force between the screw elements and the barrel. In the heating/melting zone, the temperature is at or above the melting point or glass transition temperature of the thermal binder or binders in the blend such that the conveying solids become molten as they pass through the zone. A thermal binder in this context describes an inert excipient, typically a polymer, that is solid at ambient temperature, but becomes molten or semi-liquid when exposed to elevated heat or pressure. The thermal binder acts as the matrix in which the abiraterone and other APIs are dispersed, or the adhesive with which they are bound such that a continuous composite is formed at the outlet orifice, Once in a molten state, the homogenized blend is pumped to the orifice through another heating zone that maintains the molten state of the blend. At the orifice, the molten blend may be formed into strands, cylinders or films. The extrudate that exits is then solidified typically by an air-cooling process. Once solidified, the extrudate may then be further processed to form pellets. spheres, fine powder, tablets, and the like.

A pharmaceutical formulation as disclosed herein resulting from hot melt extrusion may have a uniform shape and density and may not exhibit substantially changed solubility or functionality of any excipient. The abiraterone, excipient, or other components of the pharmaceutical formulation may lack substantial impurities.

In certain embodiments, the pharmaceutical formulation of the present disclosure prepared by hot melt extrusion may exclude a prodrug of abiraterone, such as abiraterone acetate.

In certain embodiments, the pharmaceutical formulation of the present disclosure prepared by hot melt extrusion may include a prodrug of abiraterone, such as abiraterone acetate, wherein the pharmaceutical formulation may have no solvent in the pharmaceutical formulation or a tablet thereof, and may have no impurities including the solvent in the pharmaceutical formulation or a tablet thereof.

A pharmaceutical formulation of the present disclosure may be prepared using spray drying. In the spray-drying process, components, including abiraterone, an excipient and any other APIs or excipients are dissolved in a common solvent which dissolves the components to produce a mixture. After the components have been dissolved, the solvent is rapidly removed from the mixture by evaporation in the spray-drying apparatus, resulting in the formation of a solid amorphous dispersion of the components. Rapid solvent removal is accomplished by either (1) maintaining the pressure in the spray-drying apparatus at a partial vacuum (e,g., 0,01 to 0.50 atm): (2) mixing the mixture with a warm drying gas; or (3) both (1) and (2). In addition, a portion or all of the heat required for solvent evaporation may be provided by heating the mixture.

Solvents suitable for spray-drying can be any organic compound in which the abiraterone and primary excipient and any additional APIs or excipients are mutually soluble. The solvent may also have a boiling point of 150° C. or less. In addition, the solvent should have relatively low toxicity and be removed from the dispersion to a level that is acceptable according to The international Committee on Harmonization (ICH) guidelines, which are incorporated by reference herein. A further processing step, such as tray-drying subsequent to the spray-drying process, may be used to remove solvent to a sufficiently low level.

Suitable solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethylacetamide or dimethylfoxide may also be used. Mixtures of solvents may also be used, as may mixtures with water as long as the abiraterone, excipient, and any other APIs or excipients in the pharmaceutical formulation are sufficiently soluble to allow spray-drying,

The abiraterone, excipient, or other components of a pharmaceutical formulation as disclosed herein resulting spray-drying may lack substantial impurities.

In certain embodiments, the pharmaceutical formulation of the present disclosure may be prepared by spray-drying may exclude a prodrug of abiraterone, such as abiraterone acetate.

In certain embodiments, the pharmaceutical formulation of the present disclosure may be prepared by spray-drying may include a prodrug of abiraterone, such as abiraterone acetate, wherein the pharmaceutical formulation may have no solvent in the pharmaceutical formulation or a tablet thereof, and may have no impurities including the solvent in the pharmaceutical formulation or a tablet thereof.

A pharmaceutical formulation of the present disclosure may be prepared by combining abiraterone and a cyclic oligomer excipient using other methods including but not limited to wet mass extrusion, high intensity mixing, high intensity mixing with a solvent, ball milling, ball milling with a solvent, or any solvent casting or forming process with a high mixing step, among others identifiable by skilled persons upon reading the present disclosure. The abiraterone, excipient, or other components of a pharmaceutical formulation as disclosed herein provided by any of the methods described herein or identifiable by skilled persons upon reading the present disclosure may lack substantial impurities.

In certain embodiments, the pharmaceutical formulation of the present disclosure prepared by wet mass extrusion, high intensity mixing, high intensity mixing with a solvent, ball milling, ball milling with a solvent, or any solvent casting or forming process with a high mixing step, among others may exclude a prodrug of abiraterone, such as abiraterone acetate.

In certain embodiments, the pharmaceutical formulation of the present disclosure prepared by wet mass extrusion, high intensity mixing, high intensity mixing with a solvent, ball milling, ball milling with a solvent, may include a prodrug of abiraterone, such as abiraterone acetate, wherein the pharmaceutical formulation may have no solvent in the pharmaceutical formulation or a tablet thereof, and may have no impurities including the solvent in the pharmaceutical formulation or a tablet thereof.

Following formulation of a pharmaceutical formulation as disclosed herein, an amount appropriate to provide a given unit dosage form may be further processed, for example to result in an orally administrable form. This further processing may include combining the pharmaceutical formulation as an internal phase with an external phase, if needed, along with tableting by a tableting press or encapsulation in a capsule. The external phase may include an additional amount of an excipient or a concentration enhancing polymer to further improve, for example, the therapeutic effect, bioavailability, Cₘᵢₙ, or Cₘₐₓ.

In some examples, the pharmaceutical formulation may be tableted, then coated with a composition containing another API, such as a glucocorticoid replacement API.

### C. Methods of Administering a Pharmaceutical Formulation

The FDA-approved form of crystalline abiraterone acetate, ZYTIGA®, is administered on an empty stomach to prostate cancer patients at a total dose of 1,000 mg once daily, as multiple unit dosage form tablets. The bioavailability of ZYTIGA® at these conditions is estimated to be <10%.

Recent studies have indicated that the low oral bioavailability of ZYTIGA® may be responsible for poor clinical outcomes in a significant portion of the patient population. This has been demonstrated by correlating steady-state minimum serum concentration (Cₘᵢₙ) to reductions in PSA levels. In the treatment of mCRPC with ZYTIGA®, reductions in PSA are predictive of improved clinical outcomes. Early response, such as a PSA decline>30% from baseline by 4 weeks, is associated with longer overall survival. Robust response, such as a PSA decline>50% from baseline at 12 weeks is associated with longer overall survival. However, a significant proportion of ZYTIGA® patients do not achieve robust PSA reductions.

In a Phase 3 study in chemotherapy naïve patients (COU-AA-302), 38% of subjects (208 of 542) did not achieve PSA decline>50% according to Prostate Cancer Clinical Trials Working Group (PCWG2) criteria. In a Phase 3 study in prior docetaxel treated patients (COU-AA-301), 61% of patients (632 of 790) did not achieve a PSA decline>50% according to PCWG2 criteria. In a Phase 3 study of enzalutamide in prior docetaxel treated patients (AFFIRM), patients progressing on enzalutamide were subsequently offered salvage therapy with ZYTIGA®: only 8% (3 of 37) of the patients achieved PSA decline>50%.

Better PSA response with ZYTIGA® treatment is associated with patients who have higher Cₘᵢₙ of abiraterone. In a tumor-inhibition model built upon pooled data from the COU-AA-301 and COU-AA-302 Phase 3 studies, patients with higher Cₘᵢₙ of abiraterone had longer time until PSA progression (PSA Doubling Time) which was predictive of longer overall survival. In addition, patients with lower steady state Cₘᵢₙ showed higher incidence of a negative PSA decay rate effect (acceleration of the PSA doubling time). For example, a steady state Cₘᵢₙ of 0-11 ng/mL or 12-35 ng/mL was associated with acceleration of the PSA doubling time in 24% or 22% of patients, respectively, whereas acceleration of the PSA doubling time was not observed in any patients having a steady state of Cₘᵢₙ of greater than 35 ng/mL abiraterone. However, only 5% of patients administered with ZYTIGA® in the COU-AA-301 and COU-AA-302 Phase 3 studies reached a steady state of Cₘᵢₙ of greater than 35 ng/mL abiraterone. In an FDA regulatory review analysis of COU-AA-301 trial data, subjects in the group having higher Cₘᵢₙ of abiraterone showed a trend towards longer overall survival. These results suggest that increasing Cₘᵢₙ levels by increasing overall abiraterone exposure may lead to improved clinical outcomes with abiraterone. Accordingly, in some implementations, a pharmaceutical formulation of the present disclosure may include an amount of amorphous abiraterone or a salt thereof sufficient to achieve a Cₘᵢₙ in a human patient of greater than, or greater than about, 12 ng/mL, 20 ng/mL, 30 ng/mL, 40 ng/mL, 50 ng/mL, 60 ng/mL, 70 ng/mL, 80 ng/mL, 90 ng/mL, or higher, or up to, or up to about 100 ng/mL. In some implementations, a pharmaceutical formulation of the present disclosure may include an amount of amorphous abiraterone or a salt thereof sufficient to achieve a geometric mean Cₘᵢₙ in a population of human patients of greater than, or greater than about, 12 ng/mL, 20 ng/mL, 20 ng/mL, 25 ng/mL 30 ng/mL, 35 ng/mL 40 ng/mL, 45 ng/mL 50 ng/mL, 55 ng/mL 60 ng/mL, 65 ng/mL 70 ng/mL, 75 ng/mL, 80 ng/mL, 85 ng/mL, 90 ng/mL, 95 ng/mL, or higher, or up to, or up to about 100 ng/mL.

In another study (clinicaltrials.gov: NCT01637402), patients receiving 1,000 mg per day abiraterone acetate that showed no PSA decline after 12 weeks of treatment, referred to as “primary resistant” to abiraterone acetate therapy, had lower plasma abiraterone levels than patients who did show PSA decline after 12 weeks of therapy, referred to as “responders”. Also, at the time of disease progression during standard-dose therapy (1,000 mg per day), the plasma levels of abiraterone were significantly lower in primary resistant patients compared to responders. Accordingly, as used herein, the term “resistant” in connection with abiraterone acetate therapy may refer to a patient in which therapeutic efficacy, such as PSA decline, has not been observed in response to abiraterone acetate, e.g. ZYTIGA®. In contrast, as used herein, the term “responsive” in connection with abiraterone acetate therapy may refer to a patient in which therapeutic efficacy, such as PSA decline, has been observed in response to abiraterone acetate, e.g. ZYTIGA®. In addition, as used herein the term “acquired resistant” refers to patients who previously showed a PSA decline, such as up to 50%, or more than 50%, compared to pre-treatment levels, following treatment for a time (such as at least 12 weeks) with abiraterone acetate, e.g. ZYTIGA®, but who are no longer responding to the therapy as indicated by increasing PSA.

These results suggest that increasing abiraterone plasma levels may lead to improved clinical outcomes with abiraterone. Accordingly, administration of a pharmaceutical formulation of the present disclosure may provide increased plasma levels of abiraterone in patients. Administration of a pharmaceutical formulation of the present disclosure may be associated with a decrease in resistance and/or an increase in response to the administered abiraterone, for example, a greater proportion of patients showing a decrease in PSA levels, or a longer time to disease progression, such as a greater proportion of patients showing a longer time to increase in PSA levels, and/or improvement in one or more other clinical endpoints of disease progression. Administration of a pharmaceutical formulation of the present disclosure may provide increased plasma levels of abiraterone in patients who have previously been resistant, such as primary resistant or acquired resistant, to abiraterone acetate, e.g. ZYTIGA® therapy. Administration of a pharmaceutical formulation of the present disclosure may provide increased plasma levels of abiraterone in patients who have previously been responsive to abiraterone acetate, e.g. ZYTIGA® therapy.

When patients are administered ZYTIGA® with a high fat meal, oral exposure increases substantially, with maximum serum concentration (Cₘₐₓ) and area under the plasma drug concentration-time curve (AUC) being 17 and 10-fold higher, respectively. Recent studies have indicated that this substantial food effect results from increased solubility of abiraterone acetate, such as ZYTIGA® and abiraterone in intestinal fluids of the fed state. Owing to the magnitude of this food effect and variation in meal content, ZYTIGA® must be taken on an empty stomach.

The abiraterone acetate prodrug form of abiraterone, such as ZYTIGA®, was developed to improve the solubility and bioavailability of abiraterone. However, the effectiveness of the prodrug toward improving bioavailability is limited, as evidenced by the food effect and pharmacokinetic variability cited in the label. Further, exposure was not significantly increased when the ZYTIGA® dose was doubled from 1,000 to 2,000 mg (8% increase in the mean AUC). In addition, doubling the dose of abiraterone acetate in responder patients following signs of disease progression in the NCT01637402 study was not found to have clinical benefit on disease progression. The results of these studies imply that ZYTIGA® is dosed near the absorption limit. A pharmaceutical formulation of the present disclosure may contain amorphous abiraterone, such as the active form of abiraterone, which may exhibit improved therapeutic effect, bioavailability, Cₘᵢₙ, or Cₘₐₓ as compared to an equivalent amount of crystalline abiraterone or an equivalent amount of crystalline abiraterone acetate.

A pharmaceutical formulation of the present disclosure may be administered in a dosage form, such as a unit dosage form containing an amount of abiraterone sufficient and at a frequency sufficient to achieve a greater therapeutic effect, the same or greater bioavailability, the same or greater Cₘᵢₙ, or the same or greater Cₘₐₓ as an equivalent amount of crystalline abiraterone acetate, such as ZYTIGA®, administered at the same frequency.

A pharmaceutical formulation of the present disclosure may be administered in a dosage form, such as a unit dosage form containing an amount of abiraterone sufficient and at a frequency sufficient to achieve the same or greater therapeutic effect, bioavailability, Cₘᵢₙ, or Cₘₐₓ as crystalline abiraterone acetate, such as ZYTIGA®, administered at 1000 mg once daily on an empty stomach.

For example, as illustrated in Example 16, the exemplary abiraterone pharmaceutical formulation DST-2970 IR of the present disclosure administered at a 200 mg dose provided increased abiraterone plasma levels, including increased Cₘₐₓ, and increased AUC₀₋ₜ compared to ZYTIGA® administered at 1,000 mg dose. When the results are adjusted to take into account the differences in dose between DST-2970 IR administered at 200 mg and ZYTIGA® administered at 1,000 mg, the exemplary formulation DST-2970 IR resulted in more than 6-fold increase in Cmax and more than 3-fold increase in AUC₀₋ₜ in fasted human subjects compared to ZYTIGA® administered to fasted human subjects.

Accordingly, in some implementations, administration of a pharmaceutical formulation of the present disclosure, for example in a unit dosage form, may provide an increase in Cmax in a human patient of greater than 1.5-fold, 2-fold, 3-fold, 4-fold, 5-fold, 6-fold, 7-fold, 8-fold, 9-fold, 10-fold, or 20-fold, or higher, as compared to an administration of an equivalent amount of crystalline abiraterone or crystalline abiraterone acetate, such as ZYTIGA®.

Accordingly, in some implementations, administration of a pharmaceutical formulation of the present disclosure, for example in a unit dosage form, may provide an increase in AUC₀₋ₜ in a human patient of greater than 1.5-fold, 2-fold, 3-fold, 4-fold, 5-fold, 6-fold, 7-fold, 8-fold, 9-fold, or 10-fold, or higher, as compared to an administration of an equivalent amount of crystalline abiraterone or crystalline abiraterone acetate, such as ZYTIGA®.

At 1,000 mg daily, after multiple days of dosing with ZYTIGA®, patients with mCRPC showed inter-subject variability of 79% for Cₘₐₓ and 64% for AUC₀₋₂₄ₕ. Administration of a pharmaceutical formulation of the present disclosure, for example in a unit dosage form, may result in at least a 5% decrease, at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in variability among patients with a response within two standard deviations of the average response in therapeutic effect, bioavailability, Cₘᵢₙ, or Cₘₐₓ as compared to an administration of an equivalent amount of crystalline abiraterone or crystalline abiraterone acetate, such as ZYTIGA®.

For example, as illustrated in Example 28, a pharmaceutical formulation of the present disclosure containing up to 30% by weight abiraterone may provide lower drug exposure variability (e.g, lower %CV for AUC₍₀₋₄₈ₕᵣ₎) as compared to an administration of an equivalent or greater amount of crystalline abiraterone or crystalline abiraterone acetate, such as Zytiga®.

For example, as illustrated in Example 16, the exemplary abiraterone pharmaceutical formulation DST-2970 IR of the present disclosure administered to fasted human subjects at 200 mg dose provided a 6% decrease in variability of geometric mean of Cₘₐₓ and a 4% decrease in variability of geometric mean of AUC₀₋ₜ as compared to fasted human subjects administered ZYTIGA® at 1,000 mg. In addition, the exemplary abiraterone pharmaceutical formulation DST-2970 IR of the present disclosure administered to fed human subjects at 200 mg dose provided a 25% decrease in variability of geometric mean of Cₘₐₓ and a 23% decrease in variability of geometric mean of AUC₀₋ₜ as compared to fasted human subjects administered ZYTIGA® at 1,000 mg.

Administration of ZYTIGA® with a high-fat meal increased the geometric mean of Cₘₐₓ by 17-fold and AUC_{0-∞} by 10-fold. Administration of a pharmaceutical formulation of the present disclosure, for example in a unit dosage form, may result in at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in fasting-state vs. high fat meal variability in therapeutic effect, bioavailability, Cₘᵢₙ, or Cₘₐₓ as compared to an administration of an equivalent amount of crystalline abiraterone or crystalline abiraterone acetate, such as ZYTIGA®.

For example, as illustrated in Example 16, administration of 200 mg of the exemplary abiraterone pharmaceutical formulation DST-2970 IR of the present disclosure to fed human subjects resulted in a small (about 30%) decrease in Cₘₐₓ and negligible decrease in AUC₀₋ₜ as compared to administration of 200 mg of DST-2970 IR to fasted human subjects.

Accordingly, in some implementations, administration of a pharmaceutical formulation of the present disclosure, for example in a unit dosage form, may provide less than 50%, less than 40%, less than 30%, less than 20%, less than 10% or less than 5% or less than 2% variation, such as an increase or decrease in Cₘₐₓ or AUC₋ₜwhen administered in a fed state as compared to when administered in a fasted state.

The above and other improvements may be due, at least in part, to improved solubility of abiraterone when present in a pharmaceutical formulation as of the present disclosure, as compared to the solubility of crystalline abiraterone or crystalline abiraterone acetate, such as ZYTIGA®, in other formulations.

Abiraterone is typically co-administered with a glucocorticoid replacement API, such as prednisone, methylprednisone, or prednisolone. For example, abiraterone acetate, such as ZYTIGA®, is typically co-administered with twice daily doses of 5 mg of prednisone, methylprednisone, or prednisolone. Methyprednisolone and dexamethasone may also be suitable glucocorticoid replacement APIs and may be administered in similar doses or doses calculated to achieve a similar glucocorticoid replacement effect as prednisone, methylprednisone, or prednisolone, in particular twice-daily administration of 5 mg of prednisone, methylprednisone, or prednisolone.

Abiraterone is the active metabolite of abiraterone acetate and is expected to have the same or similar biological effects as abiraterone acetate, such as ZYTIGA®, and thus may be administered with a glucocorticoid replacement API on a similar dosing schedule.

A pharmaceutical formulation of the present disclosure may further include a glucocorticoid replacement API, such as prednisone, methylprednisone, prednisolone, methylprednisolone, or dexamethasone, or other alkylated forms, along with the abiraterone and excipient or excipients.

A pharmaceutical formulation of the present disclosure may include 1000 mg of amorphous abiraterone or an amount of amorphous abiraterone sufficient to achieve the same or greater therapeutic effect, bioavailability, Cₘᵢₙ, or Cₘₐₓ in a patient as 1000 mg of crystalline abiraterone or crystalline abiraterone acetate, such as ZYTIGA®, when consumed on an empty stomach. Such a formulation may be designed for once-daily administration. Administration may be combined with co-administration of the glucocorticoid replacement API, such as prednisone, methylprednisone, prednisolone, methylprednisolone, or dexamethasone, for example twice daily.

A pharmaceutical formulation of the present disclosure may include 1000 mg of amorphous abiraterone, or an amount of amorphous abiraterone sufficient to achieve the same or greater therapeutic effect, bioavailability, Cₘᵢₙ, or Cₘₐₓ in a patient as 1000 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in a 5 mg amount. Such a formulation may be designed for once-daily administration, combined with co-administration of the glucocorticoid replacement API, such as prednisone, methylprednisone, prednisolone, methylprednisolone, or dexamethasone, for example in a 5 mg amount, once additionally daily.

A pharmaceutical formulation of the present disclosure may include 500 mg of amorphous abiraterone, or an amount of amorphous abiraterone sufficient to achieve the same or greater therapeutic effect, bioavailability, Cₘᵢₙ, or Cₘₐₓ in a patient as 500 mg of crystalline abiraterone or crystalline abiraterone acetate, such as ZYTIGA®, when consumed on an empty stomach. Such a formulation may be designed for twice-daily administration or for administration of two unit dosage forms once daily. Administration may be combined with co-administration of the glucocorticoid replacement API, such as prednisone, methylprednisone, prednisolone, methylprednisolone, or dexamethasone, for example in 5 mg amounts, for example twice daily.

A pharmaceutical formulation of the present disclosure may include 500 mg of amorphous abiraterone, or an amount of amorphous abiraterone sufficient to achieve the same or greater therapeutic effect, bioavailability, Cₘᵢₙ, or Cₘₐₓ in a patient as 500 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in 2.5 mg amounts. Such a formulation may be designed for twice-daily administration. Such a formulation may be combined with co-administration of the glucocorticoid replacement API, such as prednisone, methylprednisone, prednisolone, methylprednisolone, or dexamethasone, for example in a 5 mg amount, once additionally daily.

A pharmaceutical formulation of the present disclosure may include 250 mg of amorphous abiraterone, or an amount of amorphous abiraterone sufficient to achieve the same or greater therapeutic effect, bioavailability, Cₘᵢₙ, or Cₘₐₓ in a patient as 250 mg of crystalline abiraterone or crystalline abiraterone acetate, such as ZYTIGA®, when consumed on an empty stomach. Such a formulation may be designed for administration of two-unit dosage forms twice daily or for administration of four unit dosage forms once daily. Administration may be combined with co-administration of the glucocorticoid replacement API, such as prednisone, methylprednisone, prednisolone, methylprednisolone, or dexamethasone, for example in 5 mg amounts, for example twice daily.

A pharmaceutical formulation of the present disclosure may include 1000 mg, 500 mg, 250 mg, 200 mg, 150 mg, 100 mg, 70 mg, 50 mg, 25 mg or 10 mg of amorphous abiraterone, including ranges of 10 mg to 70 mg, 25 mg to 70 mg, or 50 mg to 70 mg, or an amount of amorphous abiraterone sufficient to achieve the same or greater therapeutic effect, bioavailability, Cₘᵢₙ, or Cₘₐₓ in a patient as 1000, 500 mg, 250 mg, 200 mg, 150 mg, 100 mg, 50 mg or 25 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in 1.25 mg amounts. Such a formulation may be designed for once-daily administration. Such a formulation may be designed for twice-daily administration. Such a formulation may be designed for three times-daily, four times-daily or more administration. Such a formulation may be combined with co-administration of the glucocorticoid replacement API, such as prednisone, methylprednisone, prednisolone, methylprednisolone, or dexamethasone, for example in a 5 mg amount, once additionally daily.

Variations of the above example formulations and dosing regimens are possible. For example, amounts of abiraterone, glucocorticoid replacement API, or both, in a pharmaceutical formulation may be varied based upon the intended administration schedule.

Although prednisone, methylprednisone, prednisolone, methylprednisolone, or dexamethasone and alkylated forms thereof are recited as specific glucocorticoid replacement APIs, other glucocorticoid replacement APIs may also be used. Combinations of glucocorticoid APIs may be used, whether in the pharmaceutical formulation of co-administered.

In general, a pharmaceutical formulation of the present disclosure may be used to administer any amount of abiraterone to a patient on any schedule.

A pharmaceutical formulation of the present disclosure may be used to administer an amount of abiraterone to a patient on a variable schedule. For example, and without limitation, such variable schedules may include, for example over a period of 28 days, any combination of daily administration frequencies such as once daily (QD), twice daily (BID), three times daily (TID) and four times daily (QID) on each of the 28 days in the period. For example, and without limitation, such variable schedules may include a combination of different doses on different days within the 28 day period, such as any of the doses disclosed herein, such as, for example, 250 mg abiraterone per day TID on days 1-3 followed by 100 mg per day QD on days 4-28 of the 28 day period, among others. Other combinations of daily administration frequencies and daily doses are identifiable by skilled persons upon reading the present disclosure.

In addition, any pharmaceutical formulation of the present disclosure may be co-administered with any other API, whether or not in the pharmaceutical formulation, that also treats prostate cancer, a side-effect of abiraterone, or a side-effect of prostate cancer. Co-administered APIs may include a glucocorticoid replacement API or another API to treat prostate cancer, such as APIs used in androgen-deprivation therapy, non-steroidal androgen receptor inhibitors, taxanes, gonadotrophin-releasing hormone antagonists, gonadotropin-releasing hormone analogs, androgen receptor antagonists, non-steroidal anti-androgens, analogs of luteinizing hormone-releasing hormone, anthracenedione antibiotics, and radiopharmaceuticals, and any combinations thereof, particularly bicalutamide, such as CASODEX® (AstraZenica, North Carolina, US) cabazitaxel, such as JEVTANA® (Sanofi-Aventis, France), degarelix, docetaxel, such as TAXOTERE® (Sanofi-Aventis), enzalutamide, such as XTANDI® (Astellas Pharma, Japan), flutamide, goserelin acetate, such as ZOLADEX® (TerSera Therapeutics, Iowa, US), leuprolide acetate, such as LUPRON® (Abbvie, Ill., US), LUPRON® DEPOT (Abbive), LUPRON® DEPOT-PED (Abbive), and VIADUR® (ALZA Corporation, California, US), mitoxantrone hydrochloride, nilutamide, such as NILANDRON® (Concordia Pharmaceuticals, Barbados), and radium 223 dichloride, such a XOFIGO® (Bayer Healthcare Pharmaceuticals, New Jersey, US), and any combinations thereof.

Amorphous abiraterone in a pharmaceutical formulation of the present disclosure, may be administered using fewer or smaller tablets or capsules than is possible with formulations crystalline abiraterone acetate, such as ZYTIGA®, which may increase patient compliance and decrease patient discomfort.

A pharmaceutical formulation of the present disclosure may be particularly useful when the patient has experienced a sub-optional response to formulations containing crystalline abiraterone acetate, such as ZYTIGA®.

A pharmaceutical formulation of the present disclosure may be useful in patients who have previously been resistant to formulations containing crystalline abiraterone acetate, such as ZYTIGA®.

A pharmaceutical formulation of the present disclosure may be useful in patients who have previously been responsive to formulations containing crystalline abiraterone acetate, such as ZYTIGA®.

A pharmaceutical formulation of the present disclosure may be administered to a patient with prostate cancer, such as a patient with castration-resistant prostate cancer, metastatic castration-resistant prostate cancer, metastatic prostate cancer, locally advanced prostate cancer, relapsed prostate cancer, or other high-risk prostate cancer.

A pharmaceutical formulation of the present disclosure may be administered to a patient with prostate cancer who has previously received treatment with chemotherapy, such as docetaxel.

A pharmaceutical formulation of the present disclosure may be administered to a patient with prostate cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with breast cancer.

A pharmaceutical formulation of the present disclosure may be administered to a patient with breast cancer who has previously received treatment with chemotherapy, such as docetaxel.

A pharmaceutical formulation of the present disclosure may be administered to a patient with breast cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with salivary gland cancer.

A pharmaceutical formulation of the present disclosure may be administered to a patient with salivary gland cancer who has previously received treatment with chemotherapy, such as docetaxel.

A pharmaceutical formulation of the present disclosure may be administered to a patient with salivary gland cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with a cancer known to respond to androgen deprivation therapy.

A pharmaceutical formulation of the present disclosure may be administered to a patient with a cancer known to respond to androgen deprivation therapy who has previously received treatment with chemotherapy, such as docetaxel.

A pharmaceutical formulation of the present disclosure may be administered to a patient with a cancer known to respond to androgen deprivation therapy who has previously received treatment with enzalutamide.

A pharmaceutical formulation of the present disclosure may be administered to a patient in combination with additional androgen-deprivation therapy.

Any of the pharmaceutical formulations maybe administered in one or more tablets.

### D. Examples

The present examples are provided for illustrative purposes only. They are not intended to and should not be interpreted to encompass the full breadth of the disclosure.

Various compositions and instruments are identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

### Example 1: Solid Dispersions of abiraterone with Various Polymer Excipients

Solid dispersions, some of which were amorphous solid dispersions and some of which were not (at the investigated processing conditions), were prepared via thermokinetic compounding using a lab-scale thermokinetic compounder (DisperSol Technologies LLC, Austin, Tex.). 10% by weight neat crystalline abiraterone was physically mixed with 90% by weight polymer excipient by hand-blending for two minutes in a polyethylene bag. Polymer excipients varied as indicated in Table 1. The binary mixture was then thermokinetically compounded with an ejection temperature of between 120° C.-230 ° C. During thermokinetic compounding, the material was subjected to a range of shear stresses controlled by a computer algorithm, with defined rotational speeds. When the ejection temperature was reached, the resulting thermokinetically processed solid dispersion (KSD) was automatically discharged into a catch tray and immediately quenched between two stainless steel plates. Thermokinetic compounding outcomes are further described in Table 1.

TABLE 1
Abiraterone-polymer excipient solid dispersions
and thermokinetic compounding outcomes

| | Composition | | |
|---|---|---|---|
| | API | Polymer | |
| Ex. No. | (10% Wt) | (90% Wt) | Outcome |

Cellulose based

| | | | |
|---|---|---|---|
| 1.1 | Abiraterone | Hydroxy Propyl Methyl | Fully Processed |
| | | Cellulose- | |
| | | METHOCEL ™ E3 | |
| 1.2 | Abiraterone | Hydroxy Propyl Methyl | Fully Processed |
| | | Cellulose- | |
| | | METHOCEL ™ E5 | |
| 1.3 | Abiraterone | Hydroxy Propyl Methyl | Fully Processed |
| | | Cellulose Acetate | |
| | | Succinate- | |
| | | AFFINISOL ® HPMCAS | |
| | | 126 G | |

Polyvinyl based

| | | | |
|---|---|---|---|
| 1.4 | Abiraterone | Polyvinyl Pyrrolidone- | Fully Processed |
| | | KOLLIDON ® 30 | |
| 1.5 | Abiraterone | Polyvinyl Acetate | Fully Processed |
| | | Phthalate- | |
| | | PHTHALAVIN ® | |
| 1.6 | Abiraterone | Polyvinyl Alcohol 4-88- | Fully Processed |
| | | EMPROVE ® | |

Acrylate based

| | | | |
|---|---|---|---|
| 1.7 | Abiraterone | Methacrylic Acid- | Fully Processed |
| | | Ethylacrylate | |
| | | copolymer- | |
| | | KOLLICOAT MAE ® | |
| | | 100-55 | |

The KSDs were further milled to a powder using a lab-scale rotor mill (IKA mill, IKA Works GmbH & Co. KG, Staufen, Germany) equipped with 20 ml grinding chamber and operated between 10000 rpm to 24000 rpm for a period of 60 seconds at a time. The milled KSDs were sieved and the particle size fraction of ≤250μm was used for further analysis.

The neat crystalline abiraterone and KSDs were analyzed for their crystalline character by XRD using a Rigaku MiniFlex 600 benchtop X-ray diffractometer (Rigaku, Inc., Tokyo, Japan). Samples were loaded into an aluminum pan, leveled with a glass slide and analyzed in the 2-theta range between 2.5°-40.0° while being spun. The step size was 0.02°, and the scanning rate was set to 5.0°/min. The following additional instrument settings were used: Slit condition: variable+fixed slit system; soller (inc.): 5.0°; IHS: 10.0 mm; DS: 0.625°; SS: 8.0 mm; soller (rec.): 5.0°; RS: 13.0 mm (Open); monochromatization: kb filter (x2); voltage: 40 kV; current: 15 mA.

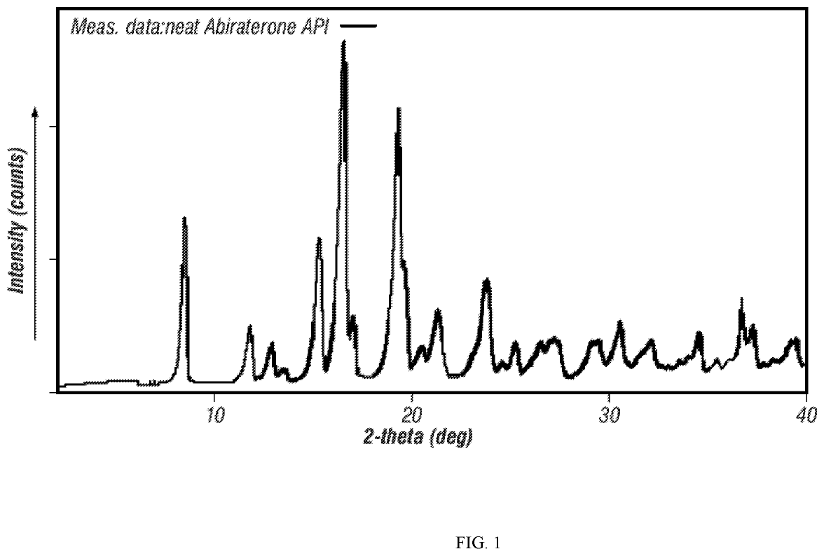

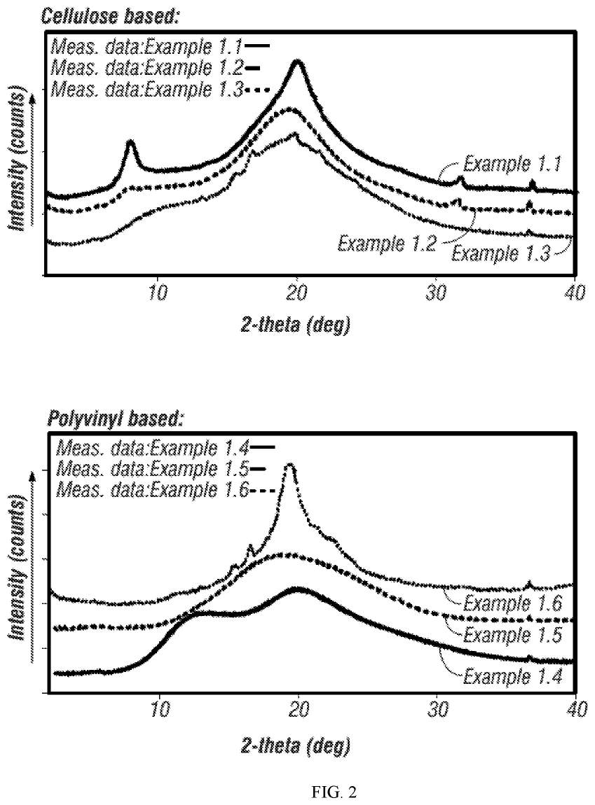

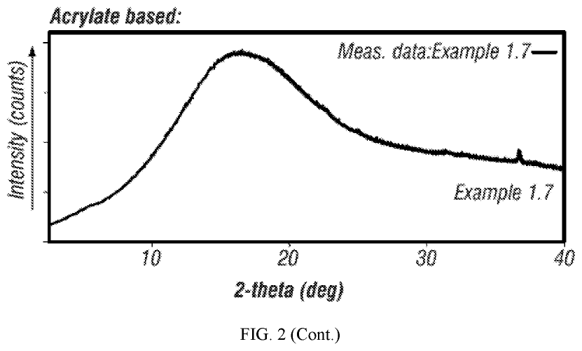

XRD diffractograms for neat crystalline abiraterone and the various KSDs are presented in FIGS. 1 and 2.

Neat crystalline abiraterone was processable via thermokinetic compounding with all three general types of polymer excipients tested. Comparing the X-ray diffractogram of neat crystalline abiraterone (FIG. 1), with X-ray diffractograms of the KSDs (FIG. 2), shows that in the cellulose-based polymer excipient group, hydroxy propyl methyl cellulose with varying viscosities yielded amorphous solid dispersions, whereas hydroxy propyl methyl cellulose acetate succinate yielded a KSD with substantially reduced crystallinity. Among the polyvinyl-based polymer excipient group, polyvinyl pyrrolidone and polyvinyl acetate phthalate produced amorphous solid dispersions, while polyvinyl alcohol 4-88 yielded a KSD with substantially reduced crystallinity. The methacrylic acid-ethylacrylate copolymer-based polymer excipient produced an amorphous solid dispersion.

### Example 2: Solid Dispersions of abiraterone with Various Cyclic Oligomer Excipients

Various KSDs of abiraterone and cyclic oligomer excipients were prepared as in Example 1. Cyclic oligomer excipients and thermokinetic compounding outcomes are described in Table 2.

TABLE 2
Abiraterone-cyclic oligomer excipient solid dispersions
and thermokinetic compounding outcomes

| | Composition | | |
|---|---|---|---|
| | API | Cyclic Oligomer | |
| Ex. No. | (10% Wt) | (90% Wt) | Outcome |

Native cyclic oligomer

| | | | |
|---|---|---|---|
| 2.1 | Abiraterone | α- Cyclodextrin- | Under processed |
| | | CAVAMAX ® W6 Pharma | |
| 2.2 | Abiraterone | β- Cyclodextrin- | Under processed |
| | | CAVAMAX ® W7 Pharma | |
| 2.3 | Abiraterone | γ- Cyclodextrin- | Under processed |
| | | CAVAMAX ® W8 Pharma | |

Modified cyclic oligomer

| | | | |
|---|---|---|---|
| 2.4 | Abiraterone | Hydroxy Propyl β | Fully processed |
| | | Cyclodextrin- | |
| | | KLEPTOSE ® HPB | |
| 2.5 | Abiraterone | Sulfo butyl β | Under processed |
| | | Cyclodextrin | |
| | | Sodium Salt- | |
| | | DEXOLVE ® 7 | |

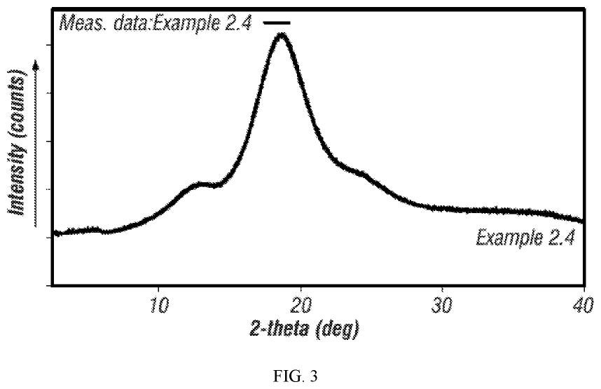

Neat crystalline abiraterone was processable via thermokinetic compounding with hydroxy propyl β cyclodextrin. Binary mixtures of neat crystalline abiraterone and all other cyclodextrins tested remained unprocessed (at the investigated processing conditions), because friction was not sufficient to obtain ejection temperature. The processed mixture was analyzed via XRD as described above in Example 1. The resulting X-ray diffractogram, shown in FIG. 3, confirmed that an amorphous solid dispersion was formed. It is expected that the other cyclodextrins tested may be processable if pre-treated by granulation or slugging, allowing sufficient friction to occur during thermokinetic compounding. Alternatively, processing these mixtures on a manufacturing-scale thermokinetic compounder may provide sufficient friction and shear to yield amorphous compositions that were not possible on the research-scale machine.

Example 3: Dissolution Testing of abiraterone Pharmaceutical Formulations

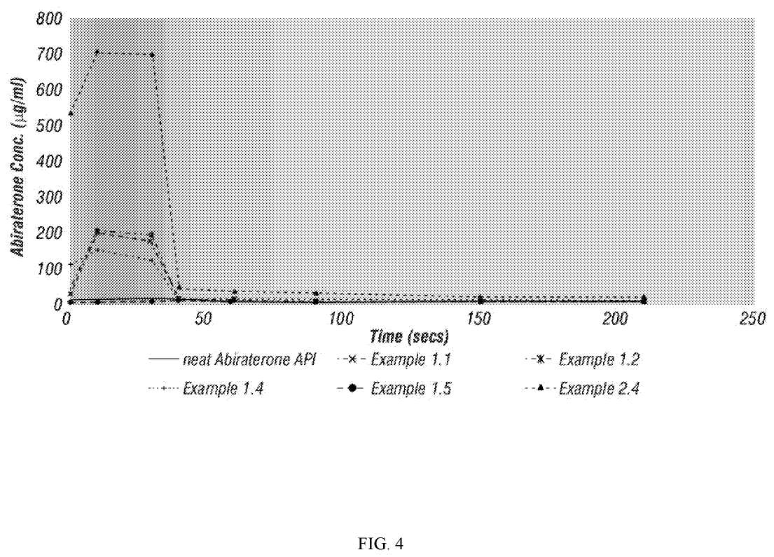

The dissolution performance of the various pharmaceutical formulations of abiraterone or neat crystalline abiraterone was analyzed using a supersaturated, non-sink, bi-phasic dissolution study. Samples equivalent to 31 mg of neat crystalline abiraterone were loaded in a dissolution vessel containing 35 ml of 0.01N HCl and placed in an incubator-shaker set to 37° C. and a rotational speed of 180 rpm. After 30 min, 35 ml of Fasted State Simulated Intestinal Fluid (FaSSIF) was added to the dissolution vessel. At set time points, samples were drawn from the dissolution vessel and centrifuged using an ultracentrifuge. The supernatants were further diluted using a diluent and analyzed by HPLC. Results are presented in FIG. 4.

Almost all of the tested pharmaceutical formulations of abiraterone with a polymer excipient or a cyclic oligomer excipient showed a higher rate and extent of dissolution as compared to neat crystalline abiraterone. Amongst the amorphous pharmaceutical formulations, the one containing a hydroxy propyl β cyclodextrin excipient showed a significantly higher extent of dissolution as compared to the pharmaceutical formulations containing a polymer excipient. This result was quite unexpected because very typically polymers are superior to all other excipients with respect to dissolution performance in ASDs formulations. Hence, it would not be predicted that a non-polymer, in this case a cyclic oligomer, would provide superior abiraterone dissolution performance, and certainly not to the extent shown in FIG. 4.

Example 4: Dissolution Testing of abiraterone Pharmaceutical Formulations with Secondary Excipients

Although the hydroxy propyl β cyclodextrin excipient provided enhanced abiraterone dissolution in the acidic phase of dissolution testing, in the neutral phase, the abiraterone precipitated owing to its weakly basic nature and substantially poorer solubility when in the unionized state. Therefore, it was hypothesized that adding a secondary excipient to the formulation could reduce the rate of precipitation in the neutral phase, thus resulting in greater overall solubility enhancement.

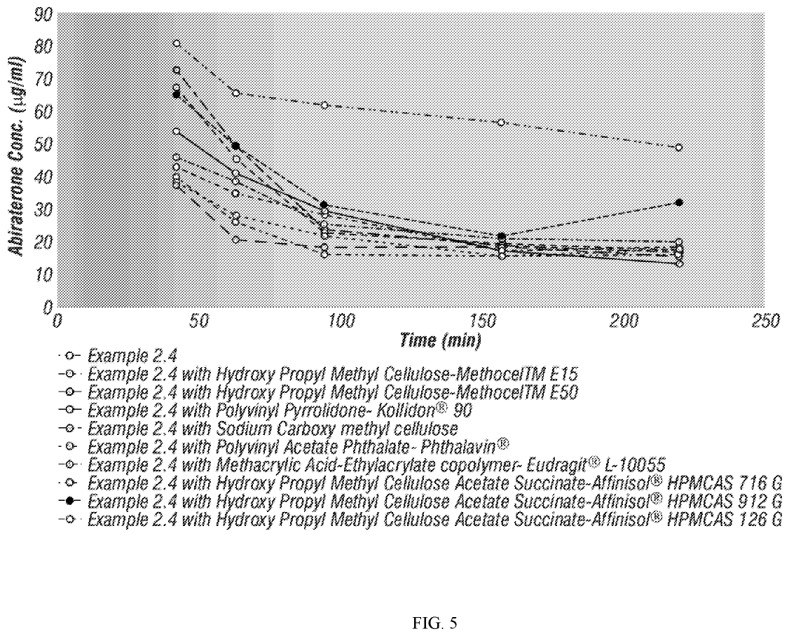

To screen secondary polymer excipients to potentially improve abiraterone dissolution in the neutral phase, an amorphous solid dispersion of 10% by weight abiraterone and 90% by weight hydroxy propyl β cyclodextrin was prepared, and samples were subjected to the acidic phase of dissolution testing using dissolution media containing 35 mg of various secondary polymers. FIG. 5 presents the results of these experiments. All secondary polymer excipients had a slight negative impact on acid phase dissolution, resulting in less than a 20% decrease in area under the dissolution curve for the relevant samples as compared to a sample with no polymer secondary excipients. In the neutral phase of the dissolution test, sodium carboxy methyl cellulose, polyvinyl acetate phthalate and hydroxy propyl methyl cellulose acetate succinate with 5-9% acetate substitution and 14-18% of succinate substitution, all had negative effects on dissolution. However, all remaining secondary excipients showed a positive impact, with hydroxy propyl methyl cellulose acetate succinate with 10-14% acetate substitution and 4-8% of succinate substitution showing the highest positive impact. This secondary polymer excipient caused a 2.4-fold increase in area under the dissolution curve during the neutral phase as compared to a sample with no polymer secondary excipient.

Example 5: Optimization of Weight Ratios of abiraterone, Cyclic Oligomer Excipient, and Secondary Excipient in Amorphous Solid Dispersions

Hydroxy propyl β cyclodextrin primary excipient concentration and hydroxy propyl methyl cellulose acetate succinate with 10-14% acetate substitution and 4-8% of succinate substitution secondary excipient concentration were optimized by subjecting various mixtures to thermokinetic compounding. Various KSDs of abiraterone and hydroxy propyl cyclodextrin primary excipient with various polymer secondary excipients were prepared as in Example 1. Relative weight percentages, excipients, and thermokinetic compounding outcomes are described in Table 3.

TABLE 3
Abiraterone-primary and secondary excipient solid
dispersions and thermokinetic compounding outcomes

| | Composition | | | |
|---|---|---|---|---|
| | | | Secondary | |
| | API | Cyclic Oligomer | Excipient | |
| Ex. No. | (% Wt) | (% Wt) | (% Wt) | Outcome |
| 3.1 | Abiraterone (10) | Hydroxy Propyl β | Hydroxy Propyl | Fully |
| | | Cyclodextrin- | Methyl Cellulose | Processed |
| | | KLEPTOSE ® HPB | Acetate Succinate- | |
| | | (50) | AFFINISOL ® | |
| | | | HPMCAS 126 G | |
| | | | (40) | |
| 3.2 | Abiraterone (10) | Hydroxy Propyl β | Hydroxy Propyl | Fully |
| | | Cyclodextrin- | Methyl Cellulose | Processed |
| | | KLEPTOSE ® HPB | Acetate Succinate- | |
| | | (60) | AFFINISOL ® | |
| | | | HPMCAS 126 G | |
| | | | (30) | |
| 3.3 | Abiraterone (10) | Hydroxy Propyl β | Hydroxy Propyl | Fully |
| | | Cyclodextrin- | Methyl Cellulose | Processed |
| | | KLEPTOSE ® HPB | Acetate Succinate- | |
| | | (70) | AFFINISOL ® | |
| | | | HPMCAS 126 G | |
| | | | (20) | |
| 3.4 | Abiraterone (10) | Hydroxy Propyl β | Hydroxy Propyl | Fully |
| | | Cyclodextrin- | Methyl Cellulose | Processed |
| | | KLEPTOSE ® HPB | Acetate Succinate- | |
| | | (80) | AFFINISOL ® | |
| | | | HPMCAS 126 G | |
| | | | (10) | |

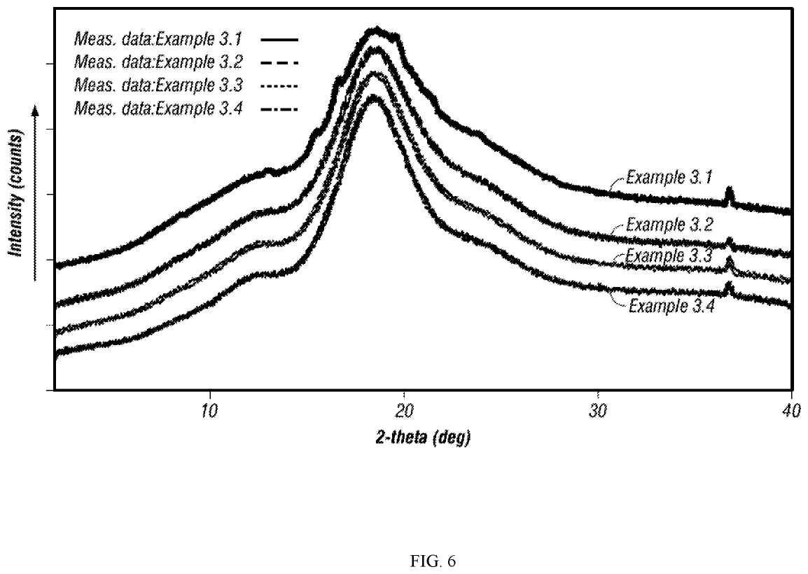

All of the ternary mixtures were processable by thermokinetic compounding. XRD, revealed that Example 3.1, which contained only 50% primary excipient, did not form an amorphous solid dispersion at explored conditions (FIG. 6). The other mixtures did form amorphous solid dispersions (FIG. 6).

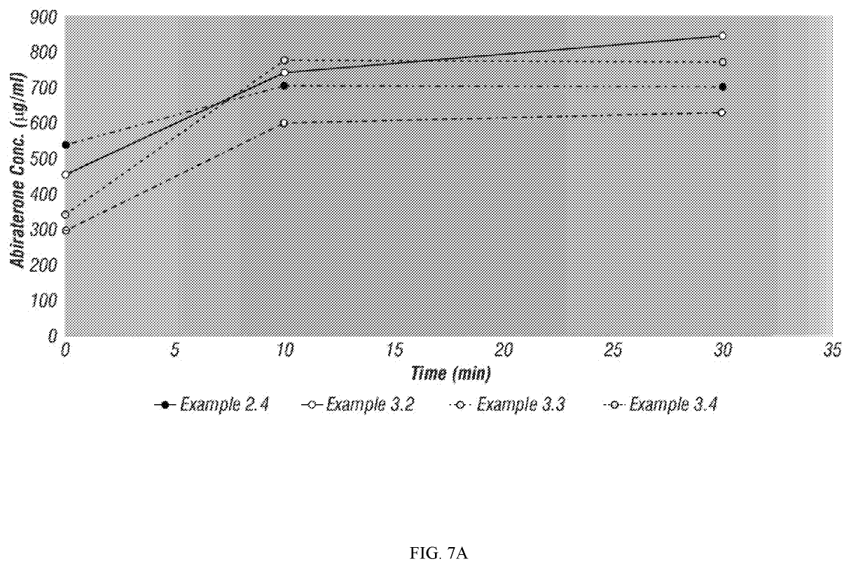

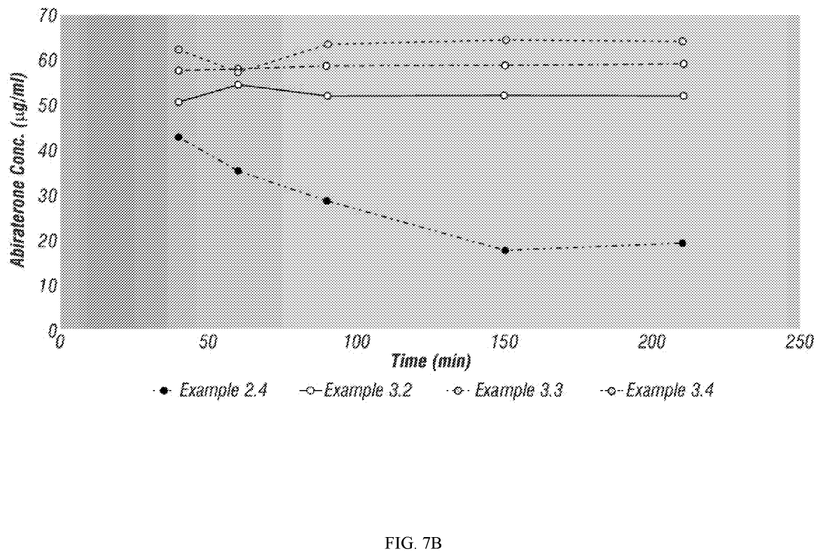

Performance evaluations of all the pharmaceutical compositions including amorphous abiraterone, hydroxy propyl β cyclodextrin and hydroxy propyl methyl cellulose acetate succinate with 10-14% acetate substitution and 4-8% of succinate substitution, were carried out similarly to the dissolution tests in Examples 3 and 4. Results are presented in FIG. 7.

In the acidic dissolution phase, the pharmaceutical formulation of Example 2.4 performed better than all other compositions evaluated (FIG. 7A). However, in neutral phase, Example 3.4, performed better than the other compositions (FIG. 7B). Similarly, overall dissolution performance was better for example 3.4 as compared to other compositions.

The results of FIG. 7A and FIG. 7B also show that, although it might be expected based on the initial tests of Example 4 that higher relative amounts of the polymer secondary excipient in the amorphous solid dispersion would lead to better dissolution enhancement, as the relative amount of polymer secondary excipient is increased, the relative amount of cyclic oligomer primary excipient decreases. This in turn disturbs the molar ratio of abiraterone to cyclic oligomer excipient, which affects dissolution performance

When amorphous abiraterone in active form and hydroxy propyl β cyclodextrin excipient are present in an amorphous solid dispersion in at weight ratio of 1:9, the molar ratio is 1:2.25. When the weight ratio decreases to 1:8, the molar ratio decreases to 1:2. Up to this point, optimal dissolution is still observed. However, when the molar ratio decreases to below 1:2, it appears that the dissolution enhancement may begin to decline.

### Example 6: Super-Saturation studies with an abiraterone-hydroxy propyl β cyclodextrin-hydroxy propyl methyl cellulose acetate succinate with 10-14% acetate Substitution and 4-8% of succinate substitution ASD

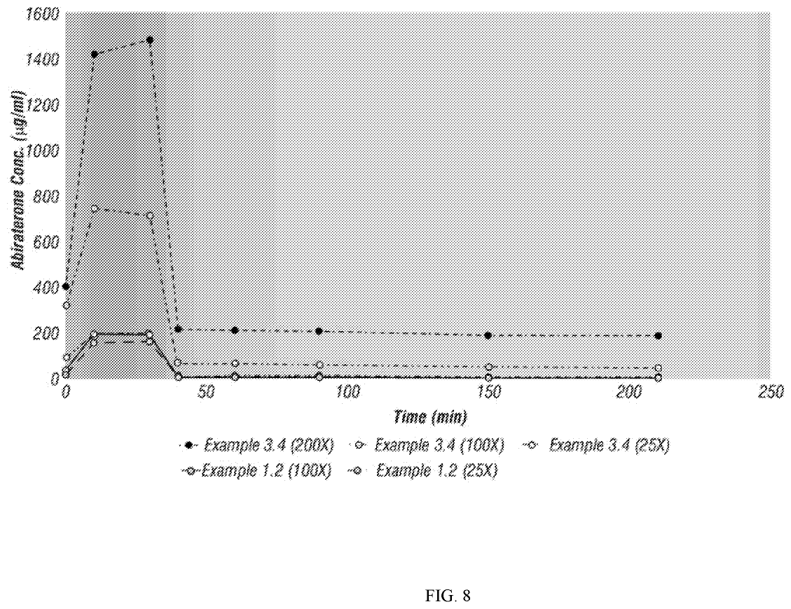

Conventionally, in a non-sink, bi-phasic dissolution study, it is expected that a pharmaceutical formulation reaches a certain degree of super-saturation for the API dissolved in the dissolution medium, at which point the addition of more of the pharmaceutical formulation to the dissolution medium does not lead to a further increase in the concentration of the API dissolved in the dissolution medium. This is considered the super-saturation threshold: the maximum amount of API that will dissolve in the dissolution media with that formulation. In order to investigate this phenomenon and determine the super-saturation threshold for abiraterone pharmaceutical formulations of the present disclosure, formulations of Example 3.4 (cyclic oligomer primary excipient with polymer secondary excipient) and Example 1.2 (polymer excipient) were tested at varying amounts. Specifically, formulations resulting in abiraterone levels of 200×(˜62 mg of abiraterone), 100×(˜31 mg of abiraterone) and 25×(˜7.7 mg of abiraterone), as compared to the intrinsic solubility of abiraterone in FaSSIF medium, were prepared. A dissolution study was carried out as in Example 3 and results are presented in FIG. 8.

For pharmaceutical formulations of Example 3.4, it was observed that as the initial loading of the composition increased from 25×, to 100× and further to 200×, the concentration of abiraterone in the dissolution medium in both the acidic phase and neutral phase increased significantly. Conversely, when the pharmaceutical formulation of Example 1.2 was evaluated at levels of 25×and 100×, only a negligible increase in concentration of abiraterone in the dissolution medium was observed. These results demonstrate that an amorphous solid dispersion containing abiraterone with a cyclic oligomer primary excipient and a polymer secondary excipient can provide enhanced dissolution and a substantially greater super-saturation threshold as compared to amorphous solid dispersions with a polymer primary excipient.

A pharmaceutical formulation of the present disclosure may result in at least 100 times, at least 200 times, at least 500 times, or at least 700 times the concentration of neat crystalline abiraterone when a 31 mg equivalent of abiraterone in the active form in the pharmaceutical formulation is added to 35 mL or 0.01N HCl.

### Example 7: Abiraterone-hydroxy propyl β cyclodextrin Pharmaceutical Formulations with increased abiraterone Loading

Abiraterone was processed with hydroxy propyl β cyclodextrin in weight ratios of 1:9, 1:4, and 3:7 by thermokinetic compounding and milled per the methods described in Example 1. The formulation details and thermokinetic compounding outcomes are described in Table 4.

TABLE 4
Abiraterone-hydroxy propyl β cyclodextrin solid dispersions
of varying drug loading and thermokinetic compounding outcomes

| | Composition | | Thermo-Kinetic |
|---|---|---|---|
| | API | Cyclic Oligomer | Processing |
| Example No. | (% Wt) | (% Wt) | Outcome |
| 2.4 | Abiraterone (10) | Hydroxy Propyl β | Fully Processed |
| | | Cyclodextrin- Kleptose ® | |
| | | HPB (90) | |
| 7.1 | Abiraterone (20) | Hydroxy Propyl β | Fully Processed |
| | | Cyclodextrin- Kleptose ® | |
| | | HPB (80) | |
| 7.2 | Abiraterone (30) | Hydroxy Propyl β | Fully Processed |
| | | Cyclodextrin- Kleptose ® | |
| | | HPB (70) | |

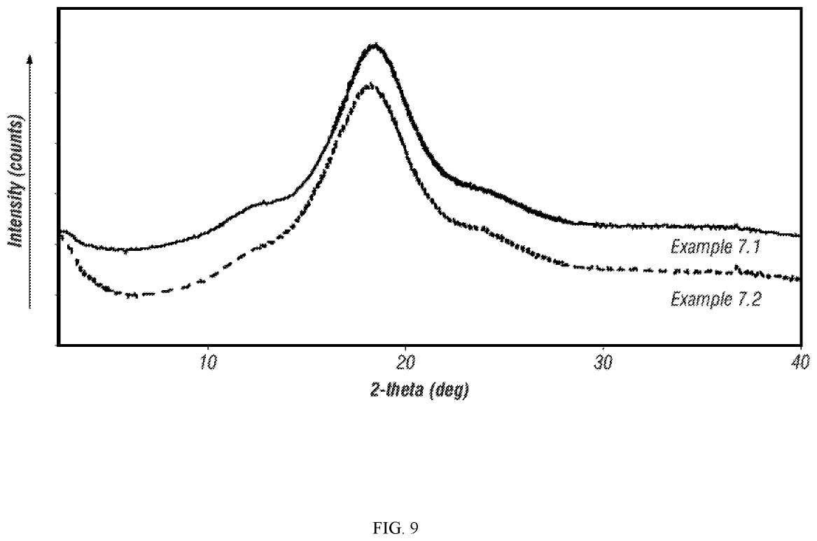

The processed formulations were analyzed via XRD by the method described in Example 1. The resulting X-ray diffractograms, shown in FIG. 9, confirmed that an amorphous solid dispersion was formed.

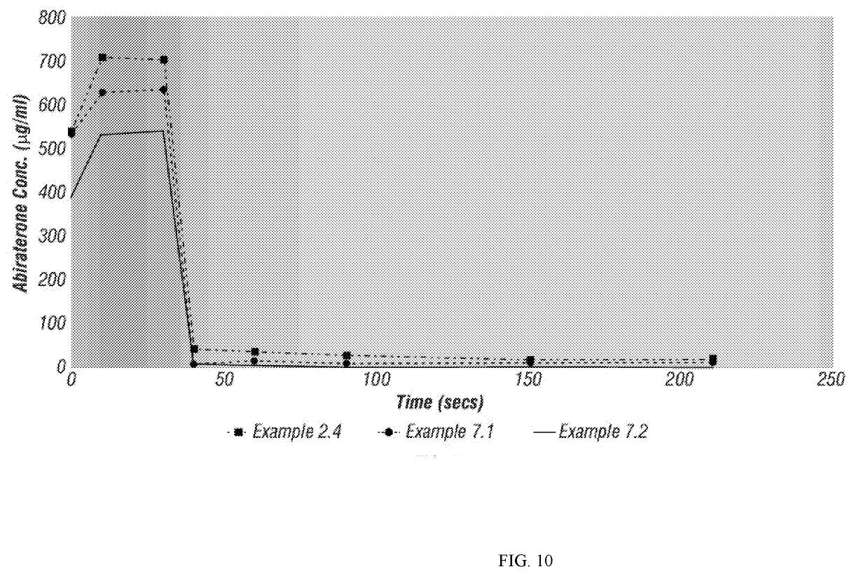

The formulations were then dissolution tested per the method of Example 3. These results are presented in FIG. 10. The dissolution results, for all formulations, show substantially enhanced solubility and dissolution properties relative to crystalline abiraterone. However, the extent of supersaturation was determined to be dependent on the abiraterone-to-hydroxy propyl β cyclodextrin ratio, with the lower ratio resulting in greater dissolution and solubility enhancement. The observation that the 1:9 weight ratio provided the best result by this dissolution test corroborates the discussion from Example 5 and conclusion that the preferred molar ratio of abiraterone-to-hydroxy propyl β cyclodextrin is greater than or equal to about 1:2.

### Example 8: Solid Dispersions of abiraterone acetate with Various Polymer Excipients

Abiraterone acetate was processed with various polymers in a 1:9 weight ratio by thermokinetic compounding and milled per the methods described in Example 1. The formulation details and thermokinetic compounding outcomes are described in Table 5.

TABLE 5
Abiraterone acetate-polymer excipient solid dispersions
and thermokinetic compounding outcomes

| | Composition | | Thermo-kinetic |
|---|---|---|---|
| | API | Polymer | Processing |
| Ex. No. | (10% Wt) | (90% Wt) | Outcome |

Cellulose based

| | | | |
|---|---|---|---|
| 8.1 | Abiraterone | Hydroxy Propyl Methyl | Fully Processed |
| | Acetate | Cellulose-Methocel ™ E5 | |
| 8.2 | Abiraterone | Hydroxy Propyl Methyl | Fully Processed |
| | Acetate | Cellulose Acetate Succinate- | |
| | | Affinisol ® HPMCAS 126 G | |
| 8.3 | Abiraterone | Hydroxy Propyl Methyl | Fully Processed |
| | Acetate | Cellulose Phthalate- | |
| | | Hypromellose Phthalate | |

Polyvinyl based

| | | | |
|---|---|---|---|
| 8.4 | Abiraterone | Polyvinyl Pyrrolidone- | Fully Processed |
| | Acetate | Kollidon ® 30 | |
| 8.5 | Abiraterone | Vinylpyrrolidone-vinyl acetate | Fully Processed |
| | Acetate | copolymer- | |
| | | Kollidon ® VA 64 | |
| 8.6 | Abiraterone | Polyethylene glycol, polyvinyl | Fully Processed |
| | Acetate | acetate and | |
| | | polyvinylcaprolactame-based | |
| | | graft copolymer- | |
| | | Soluplus ® | |

Acrylate based

| | | | |
|---|---|---|---|
| 8.7 | Abiraterone | An anionic copolymer based on | Fully Processed |
| | Acetate | methacrylic acid and ethyl | |
| | | acrylate- | |
| | | Eudragit ® L 100-55 | |

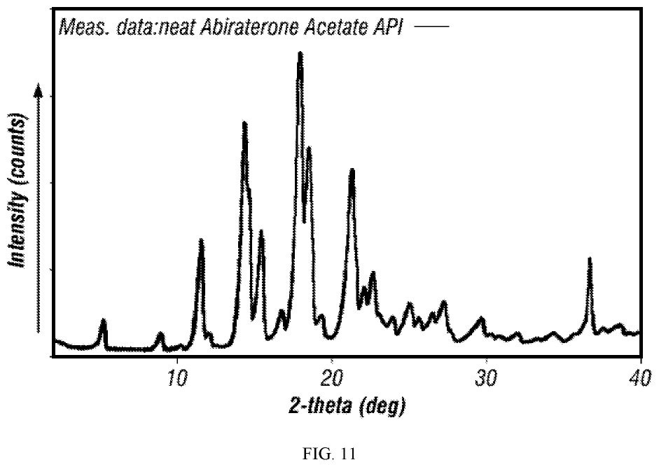

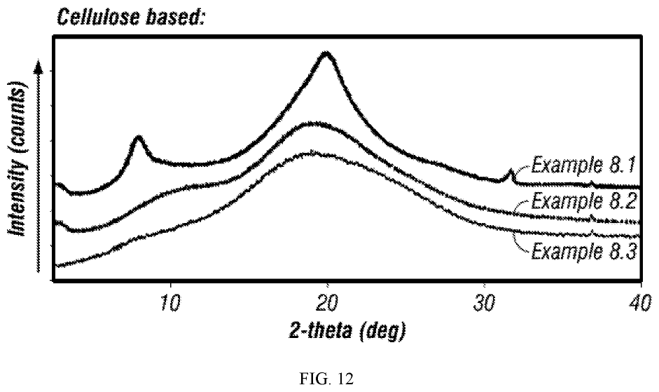

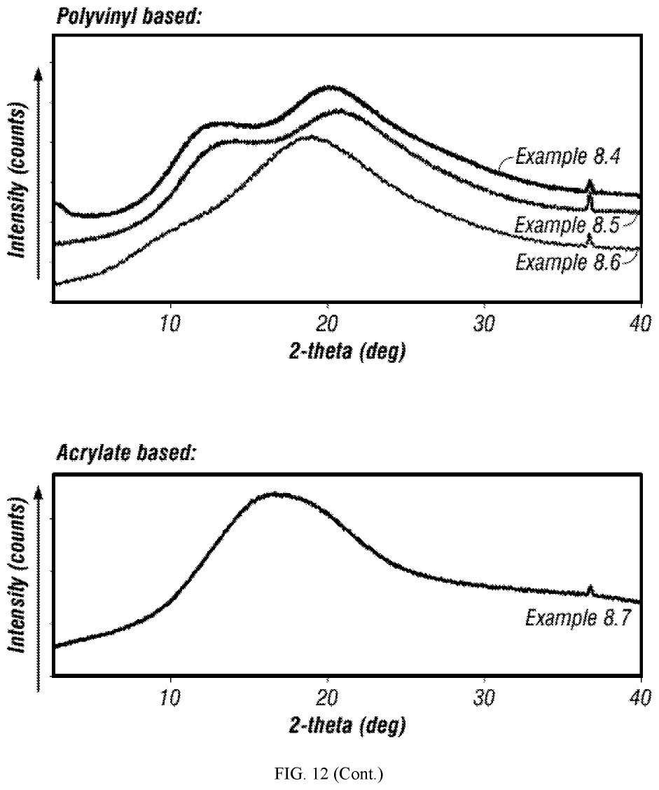

Bulk abiraterone acetate and the processed formulation were analyzed via XRD per the method described in Example 1. The resulting X-ray diffractogram for the drug substance and the processed formulations are shown in FIGS. 11 and 12, respectively. The results shown in FIG. 12 confirmed that amorphous solid dispersions of abiraterone acetate and various polymers were formed by thermokinetic compounding.

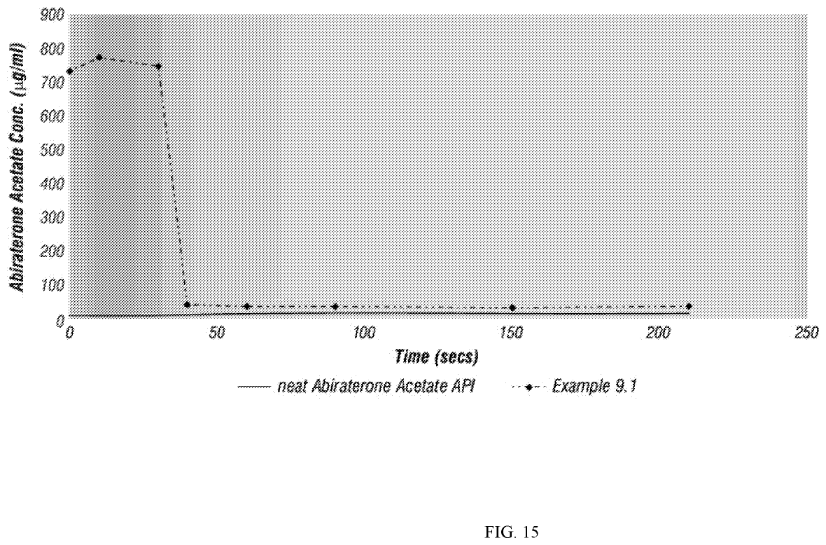

The abiraterone acetate-polymer amorphous dispersions were then dissolution tested against neat abiraterone acetate per the method of Example 3. These results are presented in FIG. 13. The dissolution results demonstrate an improvement in the rate and extent of abiraterone acetate relative to the neat drug. However, these dissolution results are inferior to dissolution results demonstrated by example 9.1 in FIG. 15.

FIG. 13 is a graph of concentration of dissolved abiraterone acetate versus time (dissolution profile) for neat crystalline abiraterone acetate or various amorphous solid dispersions of abiraterone acetate with various polymer excipients.

### Example 9: Solid Dispersions of abiraterone acetate-hydroxy propyl β cyclodextrin

Abiraterone acetate was processed with hydroxy propyl β cyclodextrin in a 1:9 weight ratio by thermokinetic compounding and milled per the methods described in Example 1. The formulation details and thermokinetic compounding outcomes are described in Table 6.

TABLE 6
Abiraterone acetate-hydroxy propyl β cyclodextrin solid
dispersion composition and thermokinetic compounding outcomes

| | Composition | | Thermo-Kinetic |
|---|---|---|---|
| | API | Cyclic Oligomer | Processing |
| Ex. No. | (% Wt) | (% Wt) | Outcome |

Modified cyclic oligomer

| | | | |
|---|---|---|---|
| 9.1 | Abiraterone | Hydroxy Propyl β | Processed |
| | Acetate (10) | Cyclodextrin- | |
| | | Kleptose ® HPB (90) | |

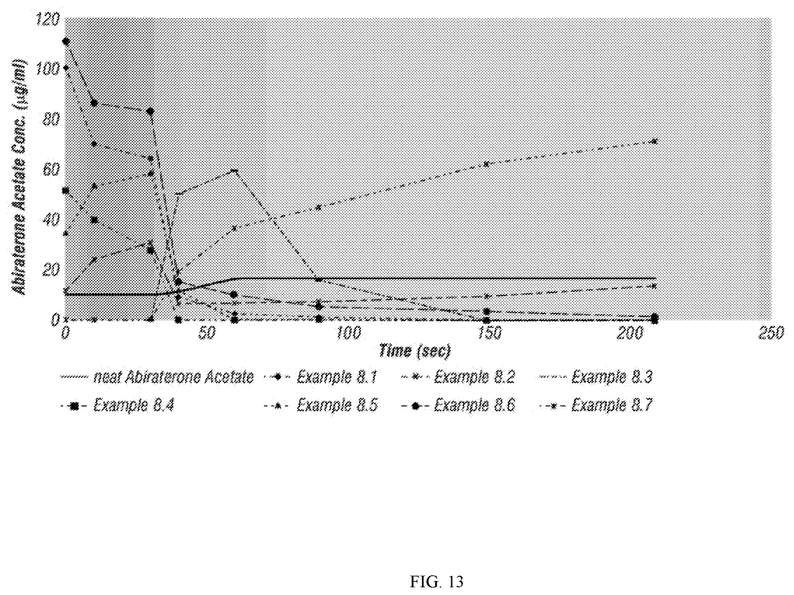

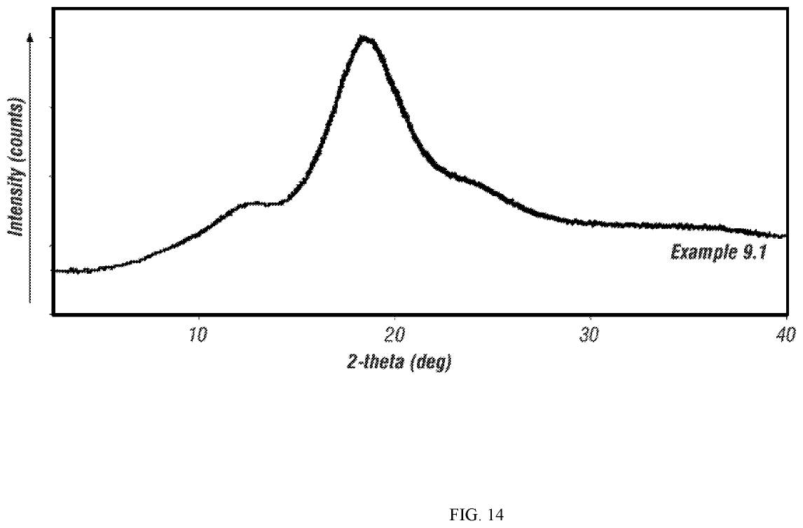

Bulk abiraterone acetate and the processed formulation were analyzed via XRD per the method described in Example 1. The resulting X-ray diffractogram for the drug substance and the processed formulations are shown in FIGS. 11 and 14, respectively. The result shown in FIG. 14 confirmed that an amorphous solid dispersion of abiraterone acetate and hydroxy propyl cyclodextrin was formed by thermokinetic compounding.

The abiraterone acetate-hydroxy propyl β cyclodextrin amorphous dispersion was then dissolution tested against neat abiraterone acetate per the method of Example 3. These results are presented in FIG. 15. The dissolution results demonstrate a substantial improvement in the rate and extent of abiraterone acetate dissolution during the acidic phase of the test for the KSD formulation relative to the neat drug. While extensive drug precipitation was observed for the KSD composition upon transition to the neutral phase of the test, the plateau drug concentration remained superior to the crystalline drug control.

Amongst the amorphous pharmaceutical formulations of abiraterone acetate, the one containing a hydroxy propyl β cyclodextrin excipient showed a significantly higher extent of dissolution as compared to the pharmaceutical formulations containing a polymer excipient. This result was quite unexpected because very typically polymers are superior to all other excipients with respect to dissolution performance in ASDs formulations. Hence, it would not be predicted that a non-polymer, in this case a cyclic oligomer, would provide superior abiraterone dissolution performance, and certainly not to the extent shown in FIG. 15.

FIG. 15 is a graph of concentration of dissolved abiraterone acetate versus time (dissolution profile) for neat crystalline abiraterone acetate and an amorphous solid dispersion of abiraterone acetate with hydroxy propyl β cyclodextrin.

### Example 10: Abiraterone acetate-hydroxy propyl β cyclodextrin Pharmaceutical Formulations with increased abiraterone acetate Loading

Abiraterone acetate was processed with hydroxy propyl β cyclodextrin in weight ratios of 1:9 and 1:4 by thermokinetic compounding and milled per the methods described in Example 1. The formulation details and thermokinetic compounding outcomes are described in Table 7.

TABLE 7
Abiraterone acetate-hydroxy propyl β cyclodextrin solid dispersions
of varying drug loading and thermokinetic compounding outcomes

| | |
|---|---|
| Composition | Thermo-Kinetic |

| | API | Cyclic Oligomer | Processing |
|---|---|---|---|
| Example No. | (% Wt) | (% Wt) | Outcome |
| 9.1 | Abiraterone | Hydroxy Propyl β | Fully Processed |
| | Acetate (10) | Cyclodextrin- Kleptose ® | |
| | | HPB (90) | |
| 10.1 | Abiraterone | Hydroxy Propyl β | Fully Processed |
| | Acetate (20) | Cyclodextrin- Kleptose ® | |
| | | HPB (80) | |

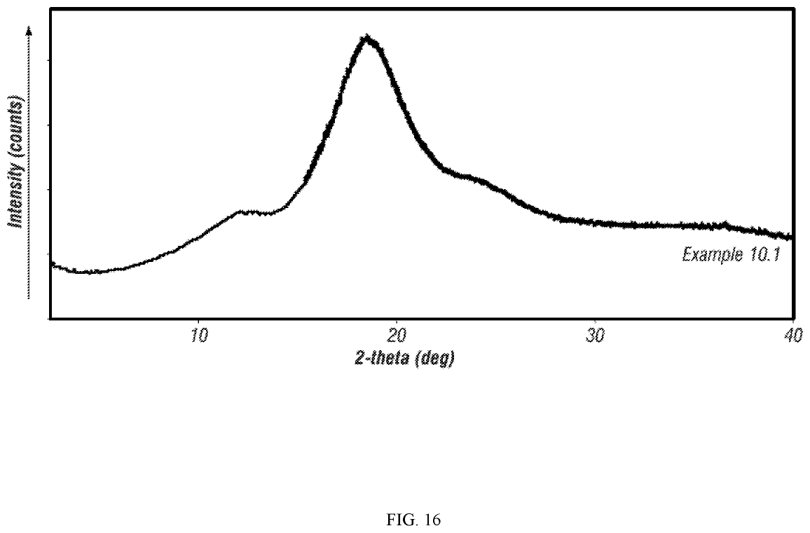

The processed formulations were analyzed via XRD by the method described in Example 1. The resulting X-ray diffractogram, shown in FIG. 16, confirmed that an amorphous solid dispersion was formed at the higher loading of abiraterone acetate.

The formulations were then dissolution tested per the method of Example 3. These results are presented in FIG. 17. The dissolution results show that the extent of supersaturation was dependent on the abiraterone acetate-to-hydroxy propyl β cyclodextrin ratio, with the lower ratio resulting in greater dissolution and solubility enhancement. The observation that the 1:9 weight ratio provided the best result by this dissolution test corroborates the discussion from Example 5 and the conclusion that the preferred molar ratio of abiraterone/abiraterone acetate-to-hydroxy propyl β cyclodextrin is greater than or equal to about 1:2.

### Example 11: Super-Saturation Studies with an abiraterone acetate-hydroxy propyl β cyclodextrin ASD

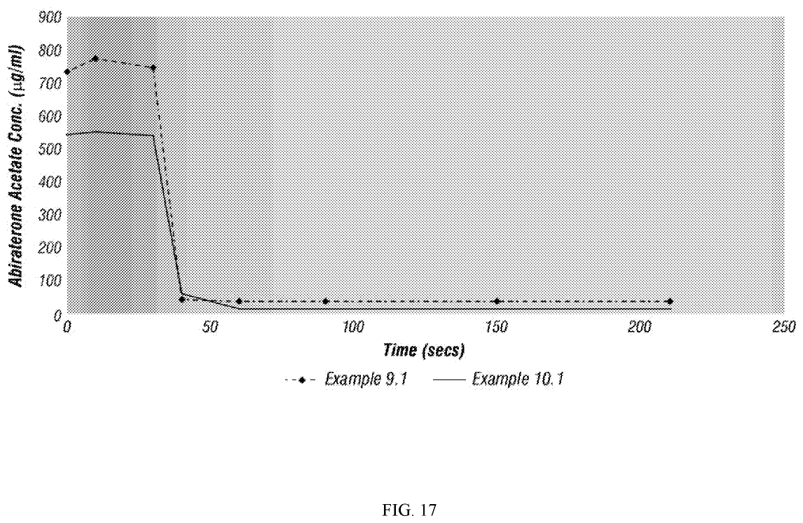

Conventionally, in a non-sink, bi-phasic dissolution study, it is expected that a pharmaceutical formulation reaches a certain degree of super-saturation for the API dissolved in the dissolution medium, at which point the addition of more of the pharmaceutical formulation to the dissolution medium does not lead to a further increase in the concentration of the API dissolved in the dissolution medium. This is considered the super-saturation threshold: the maximum amount of API that will dissolve in the dissolution media with that formulation. In order to determine the super-saturation threshold for the abiraterone acetate-hydroxy propyl β cyclodextrin (1:9) ASD of Example 9.1, the formulation was tested at concentrations varying from 400 to 100-times the intrinsic solubility of abiraterone in FaSSIF medium. A dissolution study was carried out as in Example 3 and results are presented in FIG. 18.

For a pharmaceutical formulation of Example 9.1, it was observed that as the initial loading of the composition increased from 100×, to 200×, to 300×, and finally to 400×, the concentration of abiraterone in the dissolution medium in both the acidic phase and neutral phase increased significantly. These results demonstrate that an amorphous solid dispersion containing abiraterone acetate with a cyclic oligomer excipient can provide enhanced dissolution and a substantially improved super-saturation threshold as compared to the neat drug substance.

### Example 11: Development of Immediate Release and Gastro-Retentive/Extended Release Tablets Containing ASDs of abiraterone with hydroxy propyl β cyclodextrin

In the design of a final dosage forms containing the abiraterone-cyclic oligomer amorphous solid dispersions of this disclosure, it was desired to have tablets of varying drug release rates to enable different pharmacokinetic profiles that could have unique therapeutic benefits. Therefore, an immediate release (IR) tablet was developed along with a gastro-retentive extended release (XR) tablet. Example compositions of both are provided in Table 8.

TABLE 8
Development of immediate release and gastro-retentive/extended release tablets containing
an abiraterone-hydroxy propyl β cyclodextrin amorphous solid dispersion

| | | | |
|---|---|---|---|
| | | | Example 11.2 |
| | | Example 11.1 | Gastro-retentive/ |
| | | Immediate Release | Modified/Sustained |
| Component and Quality Standard | | Tablet | Release Tablet |
| (and Grade, if applicable) | Function | % (w/w) | % (w/w) |

Drug Product Intermediate (Example 2.4)

| | | | |
|---|---|---|---|
| Abiraterone | Active | 5.00 | 5.00 |
| | Ingredient | | |
| Hydroxy Propyl β Cyclodextrin | Stabilizing | 45.00 | 45.00 |
| (Kleptose HPB) | diluent/ | | |
| | solubilizer | | |

External Phase Excipients

| | | | |
|---|---|---|---|
| Microcrystalline cellulose (Avicel PH- | Diluent/ | 24.10 | 5.61 |
| 102) | binder | | |
| Hydroxy Propyl β Cyclodextrin | Solubilizer | 5.60 | — |
| (Kleptose ® HPB) | | | |
| HPMCAS HMP grade (AQOAT ®) | Solubilizer | 3.93 | — |
| Polyethylene Oxide(Polyox WSR | Controlled | — | 24.33 |
| 301) | release | | |
| | agent | | |
| Hydroxy Propyl Methyl Cellulose | Controlled | — | 17.93 |
| (Methocel E4M) | release | | |
| | agent | | |
| Mannitol (Pearlitol 200SD) | Diluent/ | 10.37 | 1.13 |
| | binder | | |
| Croscarmallose Na (VIVASOL ®) | Disintegrant | 5.00 | 0.00 |
| Colloidal Silicon Dioxide (Aerosil | Glidant | 0.50 | 0.50 |
| 200) | | | |
| Magnesium Stearate | Lubricant | 0.50 | 0.50 |

| | | |
|---|---|---|
| Total | 100.00 | 100.00 |

The compositions shown in Table 8 were produced by blending the abiraterone-hydroxy propyl β cyclodextrin ASD powder with the tableting excipients in a suitable powder blender, then directly compressing this blend to a desired hardness with a suitable pharmaceutical tablet press.

In the case of the IR tablet, the external phase is conventional to a disintegration tablet with the exception of HPMCAS and hydroxy propyl β cyclodextrin, which are included to promote abiraterone supersaturation, particularly in the intestinal lumen.

In the case of the XR tablet, the external phase contains the functional polymers, polyethylene oxide and hydroxypropylmethyl cellulose. These polymers are incorporated into the external phase as gelling agents to promote swelling of the tablet in the stomach to: (1) facilitate retention of the tablet in the stomach and (2) modify the release of the solubility enhanced ASD form of abiraterone. This tablet design is intended to sequester the abiraterone dose in the acidic environment of the stomach where the drug is more soluble and prolong release of dissolved abiraterone in the intestinal tract such that consistent, therapeutic abiraterone exposure is achieved for the duration of therapy.

### Example 12: Dissolution Testing of Tablets Produced Per Example 11

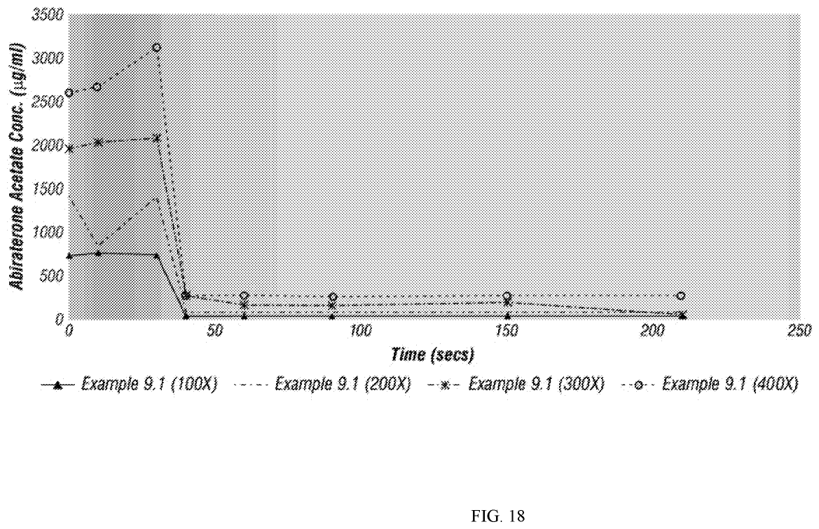

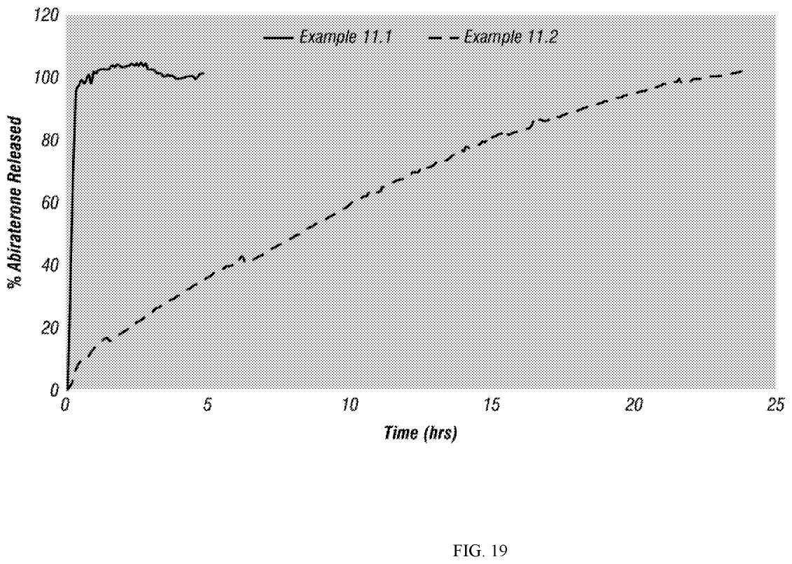

The tablets made according to Example 11 were dissolution tested to determine the rate of abiraterone release from the IR and XR dosage forms. A USP apparatus II (paddle) dissolution tester equipped with fiber-optic UV-spectroscopy for in-situ drug concentration measurements was used to conduct the analysis. Tablets of 50 mg strength were placed in dissolution vessels containing 900 ml of 0.01 N HCl heated to approximately 37° C. with a paddle stirring rate of 75 RPM. The results of this test are presented in FIG. 19.

The dissolution results shown in FIG. 19 demonstrate the rapid and complete release of abiraterone from the IR tablet of Example 11.1 and the prolonged abiraterone release over 24 hours for the XR tablet of Example 11.2. When administered to patients it is expected that the IR tablet will result in rapid and complete absorption with a high Cₘₐₓ-to-Cₘᵢₓ, ratio. Whereas, the XR tablet will result in prolonged absorption resulting in a reduced Cₘₐₓ-to-Cₘᵢₙ, ratio relative to the IR tablet and the current commercial products, i.e., Zytiga and Yonsa. This reduced Cₘₐₓ-to-Cₘᵢₙ, ratio may provide therapeutic benefit in cases were maintenance of abiraterone concentrations within the therapeutic window for the duration of treatment is critical to the therapeutic outcome. In these cases, the fast absorption and elimination of an immediate release dosage forms is undesirable because abiraterone plasma concentrations fall below the therapeutic threshold for some period of time prior to the next dose, which may promote disease progression.

### Example 13: Pharmacokinetic Testing in Male Beagle Dogs of Tablets Made per Example 11

To evaluate the in vivo performance of the IR and XR tablets presented in Example 11, the tablets (50 mg abiraterone) were orally administered to male beagle dogs along with Zytiga (250 mg abiraterone acetate) in a three-way crossover study design. Study dogs were assigned to dosing groups as shown in the Table 9. The animals received the test articles as a single oral dose. The tablet was placed on the back of the tongue, and the throat was massaged to facilitate swallowing. Then, 10-25 mL of sterile water was administered immediately via syringe to ensure the tablet was washed down into the stomach/swallowed. The first day of dose administration was designated as Day 1 of the study. For all dose events, the animals were fasted overnight and offered food at 4 hours post-dose (after the 4-hour blood collection). There was a 7-day washout between dose events.

TABLE 9
Study parameters for the pharmacokinetic evaluation
of abiraterone tablets in male beagle dogs

| | | | | Amount | | Dosing |
|---|---|---|---|---|---|---|
| Dose | Number | Dose | Test | Dosed | | Fed/Fasted |
| Group | of Animals | Event | Article | (mg) | Purpose | State |
| 1 | 5 | 1 | Example 11.1 | 50 | Test Article | Fasted |
| | | | (Abiraterone) | | | |
| | | | IR Tablets | | | |
| 1 | 5 | 2 | Example 11.2 | 50 | Test Article | Fasted |
| | | | (Abiraterone) | | | |
| | | | XR Tablets | | | |
| 1 | 5 | 3 | Zytiga | 250 | Reference | Fasted |
| | | | (Abiraterone | | Test Article | |
| | | | Acetate) Tablets | | | |

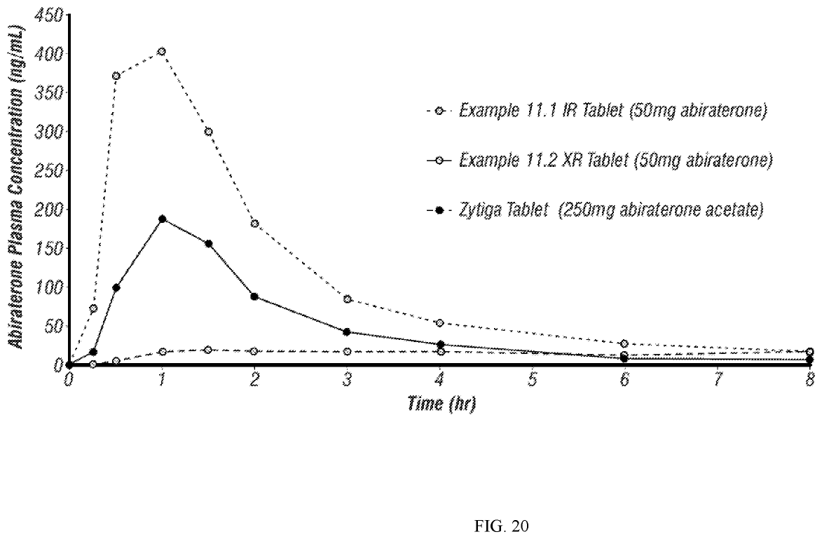

Pharmacokinetic (PK) analysis was performed comparing the IR and XR tablets to the Zytiga reference tablet. The PK parameters are presented in Table 10 and the plasma concentration versus time profiles are provided in FIG. 20. PK analysis comparing abiraterone IR tablets of Example 11.1 to Zytiga established the geometric mean ratios of dose-normalized AUC₀₋₈ and Cₘₐₓto be 14.7 and 13.9, respectively. These values indicate that the total oral exposure of abiraterone following oral administration of abiraterone IR tablets is approximately 15-fold greater than Zytiga with plasma concentrations at peak being approximately 14-fold greater. This result signifies a substantial improvement in the bioavailability of abiraterone generated by a composition of the current invention over the commercial product, Zytiga. To the inventors' knowledge, such high plasma concentrations relative to dose, as seen with the IR tablet of Example 11.1, have not been previously reported in the literature, and thus signify the uniqueness of this composition.

PK analysis comparing abiraterone XR tablets of Example 11.2 to Zytiga established the geometric mean ratios of dose-normalized AUC₀₋₈ and Cₘₐₓ to be 1.8 and 0.79, respectively.

These values indicate that the XR tablet approximately doubled total exposure (AUC) while reducing peak abiraterone plasma concentrations (Cₘₐₓ), hence decreasing the Cₘₐₓ-to-Cₘᵢₙ ratio relative to Zytiga. Given the extreme solubility challenges presented by abiraterone, particularly in the neutral pH of the intestinal lumen, such a result has not been previously achieved. It is only through the unique combination of the novel, solubility enhanced abiraterone-cyclic oligomer ASD with the hydrogel matrix of the XR tablet of this invention that such a result could be realized. Once again, the unique PK profile brought about by the drug release profile of the abiraterone XR tablet is expected to provide therapeutic benefit in cases where consistent, round-the-clock drug levels beyond the therapeutic threshold are required to achieve the desired medical outcome.

TABLE 10
Pharmacokinetic summary of Abiraterone IR and XR Tablets (50 mg abiraterone) versus Zytiga (250 mg
abiraterone acetate) in fasted male beagle dogs following administration of a single oral dose.

| | | | Dose-Normalized | Dose-Normalized | Dose-Normalized |
|---|---|---|---|---|---|
| | Tₘₐₓ | T_{1/2} | Cₘₐₓ | AUC₀₋₈ | AUCₗₐₛₜ |
| Test Article | (hr) | (hr) | (kg*ng/mL/mg) | (hr*kg*ng/mL/mg) | (hr*kg*ng/mL/mg) |
| Example 11.1 | 0.90 ± 0.42 | 5.51 ± 1.33 | 84.8 ± 17.4 | 156 ± 31.6 | 189 ± 39.9 |
| (Abiraterone) | | | 83.5 (Geo Mean) | 153 (Geo Mean) | 185 (Geo Mean) |
| IR Tablets | | | | | |
| Example 11.2 | 7.20 ± 7.53 | 3.42 ± 0.36 | 8.55 ± 9.81 | 18.9 ± 29.0 | 62.9 ± 77.1 |
| (Abiraterone) | | | 4.71 (Geo Mean) | 9.29 (Geo Mean) | 28.5 (Geo Mean) |
| GR/MR/SR/XR | | | | | |
| Tablets | | | | | |
| Zytiga | 13.35 ± 7.84 | 4.73 ± 0.51 | 30.9 ± 28.7 | 14.1 ± 12.7 | 201 ± 102 |
| (Abiraterone | | | 22.6 (Geo Mean) | 10.4 (Geo Mean) | 179 (Geo Mean) |
| Acetate) Tablets | | | | | |

¹ Average body weight adjusted dose
² Geometric mean values

Example 14: Elevated Systemic Concentrations Generated by abiraterone-cyclic Oligomer Amorphous Solid Dispersions Lead to Enhanced Tumor Regression in Xenograft Mice

To test the hypothesis that increased systemic concentrations of abiraterone results in improved tumor response, a study was conducted evaluating the efficacy of a composition made according to Example 2.4 relative to abiraterone acetate in a 22RV1 human prostate tumor xenograft model. However, prior to dosing the xenograft mice, an ascending dose PK study was conducted in non-tumored SCID mice to generate the exposure-to-dose curve of the Example 2.4 composition versus abiraterone acetate. Based on this curve, doses were selected for the xenograft study according to the observed systemic exposures.

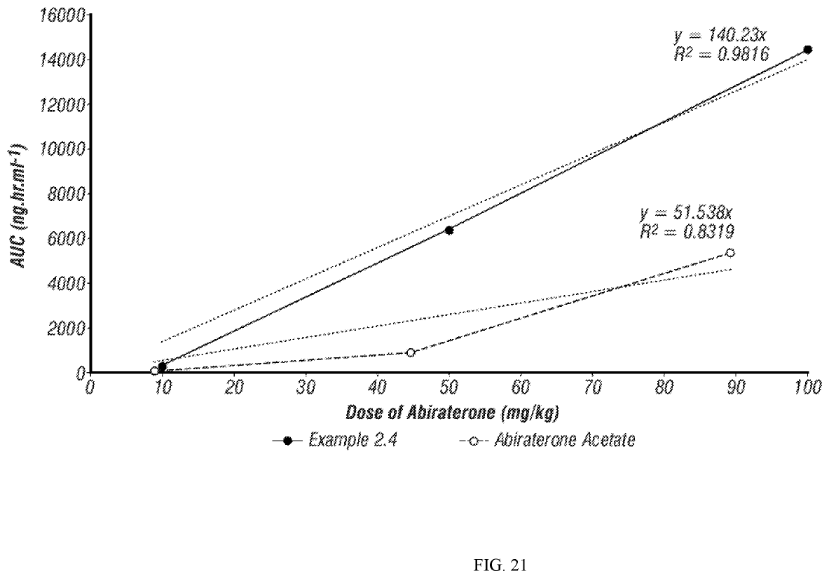

For the PK study, both test articles were dosed by oral gavage as reconstituted powders in an aqueous suspension vehicle. All animals were fasted overnight prior to dosing. The study parameters are summarized in Table 11 and the resulting exposure versus dose curve is presented in FIG. 21.

TABLE 11
Study parameters from the ascending dose study in SCID mice comparing
the pharmacokinetics of Example 2.4 to abiraterone acetate.

| | | | | Dose | | |
|---|---|---|---|---|---|---|
| | Number of | Abiraterone | Dose Level | Concentration | Dose Volume | |
| Group | Males | Formulation | (mg/kg) | (mg/mL) | (mL/kg) | Route |
| 1 | 24 | Example 2.4 | 10 | 1 | 10 | PO |
| 2 | 24 | | 50 | 5 | | |
| 3 | 24 | | 100 | 10 | | |
| 4 | 24 | Abiraterone | 10 | 1 | | |
| 5 | 24 | Acetate | 50 | 5 | | |
| 6 | 24 | | 100 | 10 | | |

The dose-exposure curve shown in FIG. 21 reveals that the dose linearity and total exposure achieved with the Example 2.4 composition is superior to abiraterone acetate. Specifically, the AUC ratio of Example 2.4 to abiraterone acetate at the low, middle, and high doses were 4.0, 7.23, and 2.6, respectively. A linear trendline was fit to both exposure versus dose curves in order to calculate the appropriate xenograft study doses based upon patient exposure data taken from the Zytiga label. From this analysis, low and high doses of abiraterone acetate were determined to be 22.4 and 100 mg/kg, and the corresponding doses for the Example 2.4 composition were 20 mg/kg and 89.2 mg/kg.

The objective of the xenograft mice study was to determine the anti-tumor activity of a composition made per Example 2.4 as a single agent versus abiraterone acetate in the 22RV1 human prostate tumor xenograft model. The study was conducted in CB.17 SCID mice injected with 22RV1 cells (5×10⁶ cells/mouse) in the subcutaneous right flank. Tumors were grown to a mean tumor size between 100 and 150 mm³ prior to study enrollment. The mice were dosed with the test article and reference once-daily by oral gavage per Table 12. Tumor volume was measured throughout the study. The study was terminated on day 26 when mean tumor volume of two experimental groups reached≥1500 mm³.

TABLE 12
Dosing parameters of the anti-tumor study in 22RV1 xenograft mice.

| | | | Vehicle | Abiraterone | |
|---|---|---|---|---|---|
| | | | Control | Acetate | DST-2970 |
| Group | | N | (QD to End) | (QD to End) | (QD to End) |
| 1. | Vehicle Control (PO) | 10 | X | | |
| 2. | Abiraterone Acetate Dose #1 22.4 mg/kg | 10 | | X | |
| | (PO) | | | | |
| 3. | Abiraterone Acetate Dose #2 100 mg/kg | 10 | | X | |
| | (PO) | | | | |
| 4. | Example 2.4 Dose #1 20 mg/kg (PO) | 10 | | | X |
| 5. | Example 2.4 Dose #2 89.2 mg/kg (PO) | 10 | | | X |

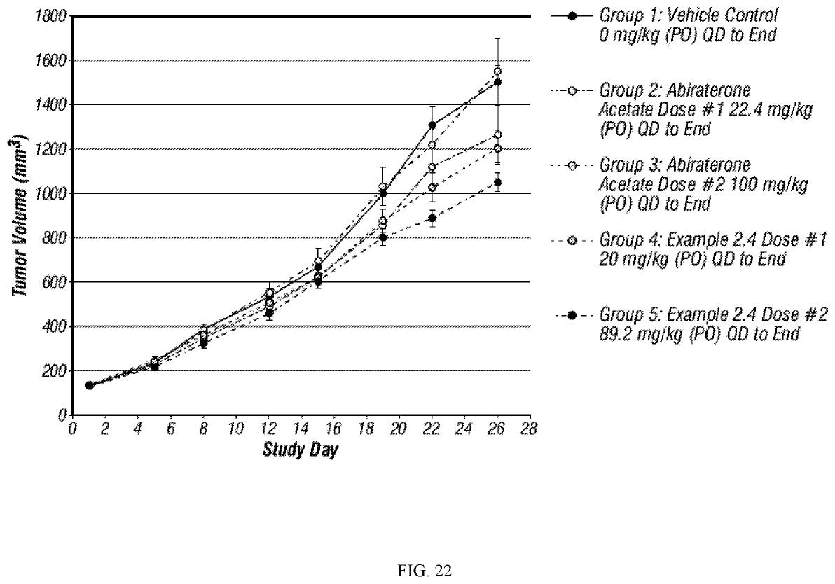

The study results are provided in FIG. 22 and Table 13. The results show that treatment with the Example 2.4 composition showed statistically significant reductions in tumor growth relative to vehicle control at the low (p=0.014) and high (p<0.001) doses. Conversely, treatment with abiraterone acetate did not result in statistically different tumor growth relative to vehicle control.

TABLE 13
Tumor growth results following once-daily administration of abiraterone acetate
or composition from Example 2.4 at two dose levels to 22RV1 xenograft mice.

| | | | | Mean | | | | | |
|---|---|---|---|---|---|---|---|---|---|
| | | | | Tumor | | Median | | | |
| | | | | Volume on | | Tumor | | | Student's |
| | | | | day 26 | | Volume | | Std. | t-test |
| Group | Compound | Dose | | (mm3) | % T/C | (mm3) | % T/C | Error | p-value |
| 1 | Vehicle | 0 | mg/kg | 1502.6 | | 1558.5 | | 75.00 | |
| | Control | | | | | | | | |
| 2 | Abiraterone | 22.4 | mg/kg | 1264.2 | 84.1 | 1135.0 | 72.8 | 129.95 | 0.165 |
| | Acetate | | | | | | | | |
| | Dose #1 | | | | | | | | |
| 3 | Abiraterone | 100 | mg/kg | 1549.4 | 103.1 | 1471.5 | 94.4 | 149.64 | 0.757 |
| | Acetate | | | | | | | | |
| | Dose #2 | | | | | | | | |
| 4 | Example 2.4 | 20 | mg/kg | 1203.8 | 80.1 | 1199.8 | 77.0 | 67.51 | 0.014 |
| | Dose #1 | | | | | | | | |
| 5 | Example 2.4 | 89.2 | mg/kg | 1048.4 | 69.8 | 1032.9 | 66.3 | 41.16 | p < 0.001 |
| | Dose #2 | | | | | | | | |

Prophetic Example 15: Therapeutic Efficacy in Cancers and Other Conditions Responsive to androgen Suppression

These result in Example 14 clearly demonstrate that the increased systemic abiraterone concentrations achieved by the compositions disclosed herein lead to superior anti-tumor response relative to abiraterone acetate. The in vivo systemic abiraterone exposures observed following oral administration of compositions disclosed herein (on a per dose basis) are believed to be the highest published to date; therefore, the Inventors believe this anti-tumor response to be unprecedented. Extrapolating from this result to human patients gives indication that the compositions of the current invention could provide superior therapeutic efficacy to patients with cancers that respond to androgen suppression, such as, prostate and breast cancers.

For example, it is contemplated that increased therapeutic efficacy associated with the pharmaceutical formulations of the present disclosure will be achieved in cancers and other conditions associated with cytochrome P450 17A1 (CYP17A1), also known as steroid 17α-monooxygenase, 17α-hydroxylase, 17,20-lyase, or 17,20-desmolase, or responsive to inhibitors of CYP17A1.

For example, it is contemplated that increased therapeutic efficacy associated with the pharmaceutical formulations of the present disclosure will be achieved in prostate cancers including but not limited to castration-resistant prostate cancer, idiopathic prostate cancer, prostate cancer associated with obesity, prostate cancer associated with elevated plasma levels of testosterone, prostate cancers linked with mutations in BRCA1 or BRCA2 genes, or other prostate cancer-linked genes such as the Hereditary Prostate cancer gene 1 (HPC1), the androgen receptor, and the vitamin D receptor genes, TMPRSS2-ETS gene family fusions such as TMPRSS2-ERG or TMPRSS2-ETV1/4, or single-nucleotide polymorphisms (SNPs) linked with prostate cancer such as those identifiable by skilled persons including those described by Eeles RA, et al. (2008) Nature Genetics. 40 (3): 316-321; Thomas G, et al. (2008) Nature Genetics. 40 (3): 310-315, incorporated herein by reference, among others.

For example, it is contemplated that increased therapeutic efficacy associated with the pharmaceutical formulations of the present disclosure will be achieved in breast cancers including but not limited to triple negative breast cancer. A diagnosis of “triple negative breast cancer” means that the three most common types of receptors known to fuel most breast cancer growth (estrogen, progesterone, and the HER-2/neu gene) are not present in the cancer tumor.

For example, it is contemplated that increased therapeutic efficacy associated with the pharmaceutical formulations of the present disclosure will be achieved in other breast cancers including but not limited to estrogen receptor-positive, progesterone-receptor positive, and/or HER-2-receptor positive breast cancers.

For example, it is contemplated that increased therapeutic efficacy associated with the pharmaceutical formulations of the present disclosure will be achieved in breast cancers such as idiopathic breast cancers or breast cancers associated with gene mutations including but not limited to those associated with gene mutation in BRCA1 or BRCA2 , p53 (e.g., in Li-Fraumeni syndrome), PTEN (e.g., in Cowden syndrome), STK11 (e.g., in Peutz-Jeghers syndrome), CHEK2, ATM, BRIP1, and PALB2, among others identifiable by skilled persons upon reading the present disclosure.

For example, it is contemplated that increased therapeutic efficacy associated with the pharmaceutical formulations of the present disclosure will be achieved in other breast cancers including but not limited to triple-negative androgen receptor positive locally advanced/metastatic breast cancer or ER-positive HER2-negative breast cancer or ER positive metastatic breast cancer or apocrine breast cancer, among others identifiable by skilled persons upon reading the present disclosure.

For example, it is contemplated that increased therapeutic efficacy associated with the pharmaceutical formulations of the present disclosure will be achieved in other cancers such as Cushing's syndrome with adrenocortical carcinoma, urothelial carcinoma or bladder cancer or urinary bladder neoplasms, androgen receptor expressing, relapsed/metastatic, salivary gland cancer or recurrent and/or metastatic salivary gland cancer or salivary glands tumors or salivary duct carcinoma, among others identifiable by skilled persons upon reading the present disclosure.

It is also contemplated that increased therapeutic efficacy associated with the pharmaceutical formulations of the present disclosure will be achieved in non-cancer androgen-dependent conditions including but not limited to acne, seborrhea, androgenic alopecia, hirsutism, hidradenitis suppurativa, precocious puberty in boys, hypersexuality, paraphilias, benign prostatic hyperplasia (BPH), and hyperandrogenism in women such as in polycystic ovary syndrome (PCOS), among others.

Example 16: Pharmacokinetic Testing in Human Subjects Administered with IR abiraterone Tablets Made per Example 11.1 Compared to ZYTIGA®

Healthy male human subjects (n=24) were enrolled in a trial to assess abiraterone pharmacokinetics following oral administration of 200 mg per day IR abiraterone made according to Example 11.1 (herein also referred to as DST-2970 IR), compared to oral administration of 1,000 mg per day abiraterone acetate (ZYTIGA®). Subjects received the following treatments: (1) 200 mg DST-2970 IR, fed state; (2) 200 mg DST-2970 IR, fasted state; or (3) 1,000 mg ZYTIGA®, fasted state.

200 mg per day DST-2970 IR was administered as 4×50 mg IR tablets and ZYTIGA® was administered as 2×500 mg film-coated tablets.

The study followed a single center, randomized, single dose, laboratory-blinded, 3-period, 6-sequence, crossover design. Subjects were randomized to a treatment sequence as shown in Table 14.

TABLE 14
Treatment sequence.

| | Period 1 | Period 2 | Period 3 |
|---|---|---|---|
| Sequence ABC | Treatment-1 | Treatment-2 | Treatment-3 |
| Sequence BCA | Treatment-2 | Treatment-3 | Treatment-1 |
| Sequence CAB | Treatment-3 | Treatment-1 | Treatment-2 |
| Sequence CBA | Treatment-3 | Treatment-2 | Treatment-1 |
| Sequence ACB | Treatment-1 | Treatment-3 | Treatment-2 |
| Sequence BAC | Treatment-2 | Treatment-1 | Treatment-3 |

wherein Treatment-1 = A; Treatment-2 = B; Treatment-3 = C.

There was a wash-out of 7 days between drug administrations, corresponding to about 12 times the expected half-life of abiraterone.

In each study period, abiraterone was administered as a single oral dose with approximately 240 mL of water, in the morning, following a 10-hour overnight fast, with food or in the fasted state according to treatment assignment.

In each study period, 21 blood samples were collected for PK assessments. The first blood sample was collected prior to drug administration while the others were collected up to 72 hours after drug administration.

Abiraterone plasma concentrations were measured by a validated bioanalytical method.

Statistical analysis of all PK parameters was based on an ANOVA model. Two-sided 90% confidence interval of the ratio of geometric least-squares means (LSmeans) was obtained from natural logarithm-transformed (1n-transformed) PK parameters including Cmax (maximum observed concentration) and AUC₀₋ₜ (cumulative area under the concentration time curve calculated from time 0 to time of last observed quantifiable concentration, using the linear trapezoidal method).

A comparative bioavailability assessment was performed as follows. Statistical inference of abiraterone from DST-2970 IR and Zytiga® was based on a bioequivalence approach using the following standards: the ratio of geometric LSmeans with corresponding 90% confidence interval calculated from the exponential of the difference between the Treatment-1 and Treatment-3 and between Treatment-2 and Treatment-3 for the ln-transformed parameters Cₘₐₓ and AUC_{0-T}.

A food effect assessment was performed as follows. The effect of a high-fat meal on the bioavailability of abiraterone from DST-2970 IR was determined by comparing the Cₘₐₓ and AUC_{0-T} obtained under fasted and fed conditions after administration of DST-2970 IR.

The formula to estimate the intra-subject coefficient of variation (CV) was: \sqrt{e^{MSE}−1}, where MSE is the Mean Square Error obtained from the ANOVA model of the ln-transformed parameters.

Pharmacokinetic parameters of Cₘₐₓ, dose-adjusted Cₘₐₓ, AUC₀₋ₜ and dose-adjusted AUC₀₋ₜwere calculated. Dose adjusted values were obtained by dividing the PK parameter by the abiraterone dose in mg. Zytiga® contains abiraterone acetate, so the amount of abiraterone in 1,000 mg of Zytiga® is 892.3 mg.

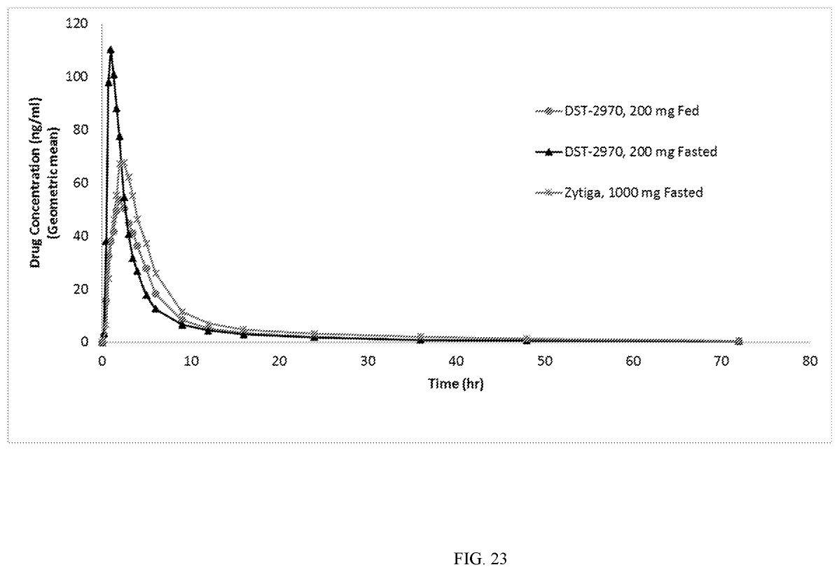

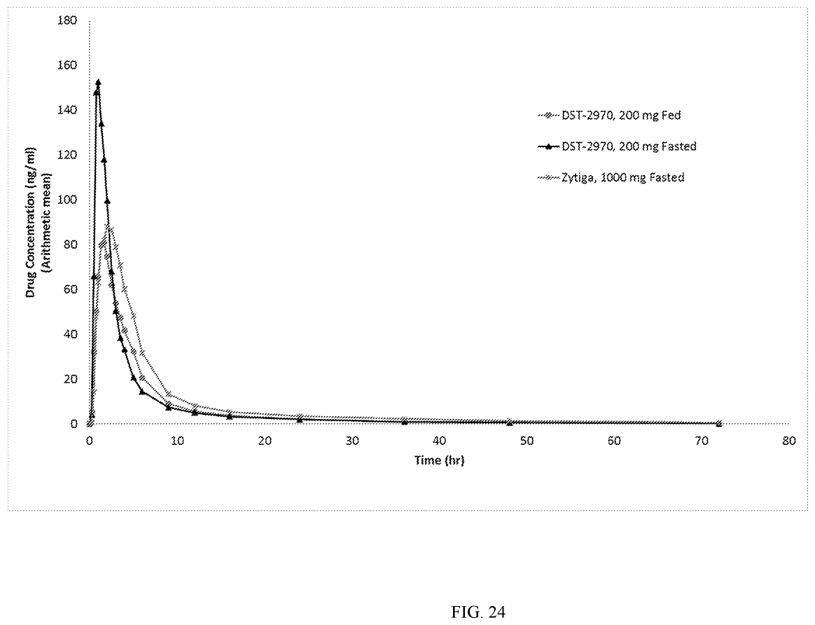

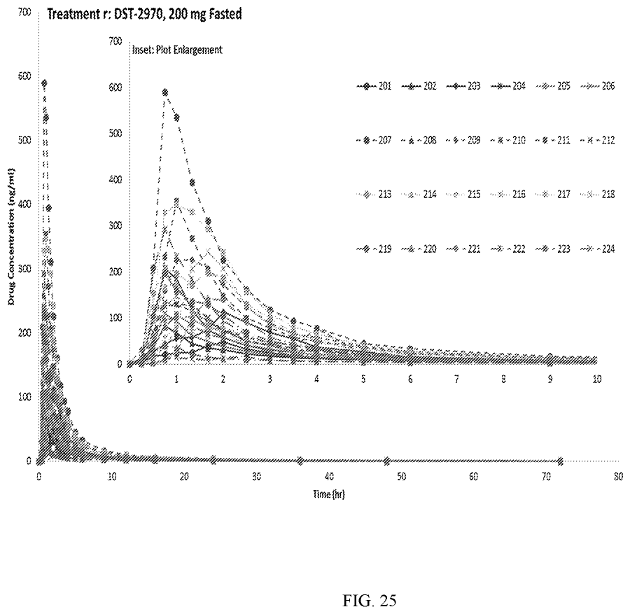

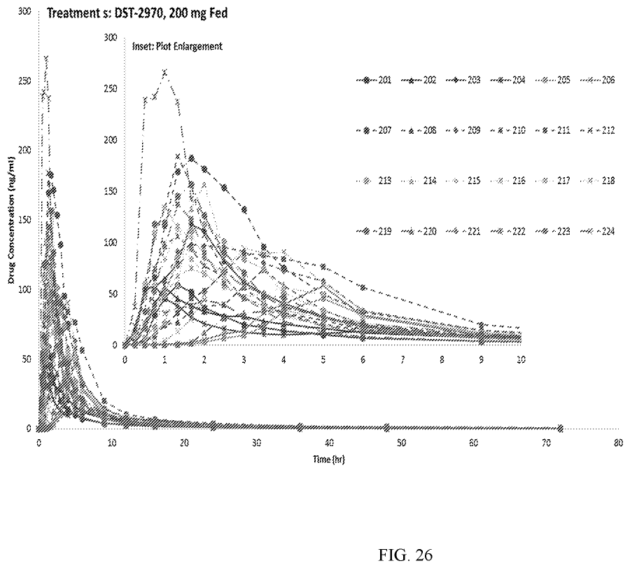

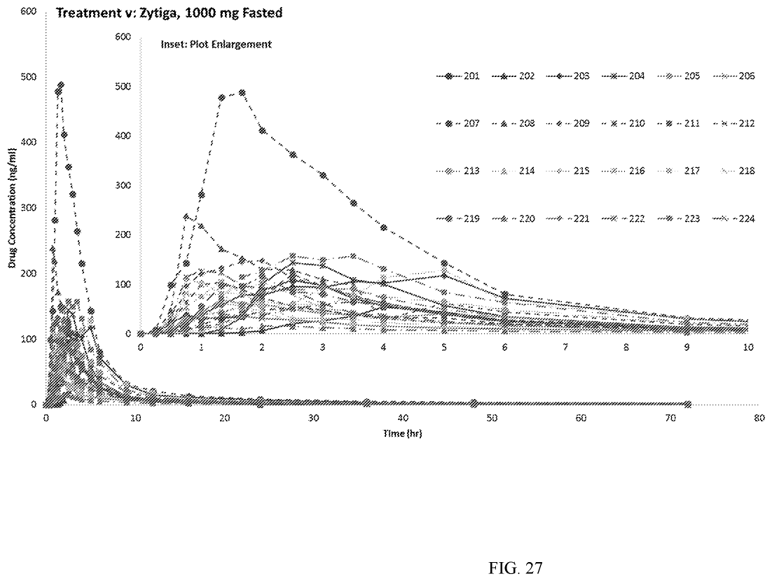

FIGS. 23-27 and Tables 15 to 18 report the results of the study.

TABLE 15
Abiraterone Cmax in human subjects following oral administration
of 200 mg abiraterone DST-2970 IR (fed or fasted) or 1,000
mg abiraterone acetate ZYTIGA ® (fasted).

| Group | N | Geo. Mean | % CV |
|---|---|---|---|
| Zytiga, fasted | 24 | 97.86 | 0.76% |
| IR Fasted | 24 | 139.27 | 0.70% |
| IR Fed | 24 | 94.29 | 0.51% |

TABLE 16
Dose-adjusted abiraterone Cmax in human subjects following
oral administration of 200 mg abiraterone DST-2970 IR (fed
or fasted) or 1,000 mg abiraterone acetate ZYTIGA ® (fasted).

| | | | F |
|---|---|---|---|
| | | | (relative to |
| Group | N | Geo. Mean | ZYTIGA ®) |
| Zytiga, fasted | 24 | 0.110 | — |
| IR Fasted | 24 | 0.696 | 6.4 |
| IR Fed | 24 | 0.471 | 4.3 |

wherein “F” indicates bioavailability.

TABLE 17
Abiraterone AUC₀₋ₜ in human subjects following oral administration
of 200 mg abiraterone DST-2970 IR (fed or fasted) or 1,000
mg abiraterone acetate ZYTIGA ® (fasted).

| Group | N | Geo. Mean | % CV |
|---|---|---|---|
| Zytiga, fasted | 24 | 518.25 | 0.63% |
| IR Fasted | 24 | 400.86 | 0.59% |
| IR Fed | 24 | 393.29 | 0.40% |

TABLE 18
Dose-adjusted abiraterone AUC₀₋ₜ in human subjects following
oral administration of 200 mg abiraterone DST-2970 IR (fed
or fasted) or 1,000 mg abiraterone acetate ZYTIGA ® (fasted).

| | | | F |
|---|---|---|---|
| | | | (relative to |
| Group | N | Geo. Mean | ZYTIGA ®) |
| Zytiga, fasted | 24 | 0.581 | — |
| IR Fasted | 24 | 2.00 | 3.5 |
| IR Fed | 24 | 1.97 | 3.4 |

The results show that, in human subjects, the exemplary abiraterone pharmaceutical formulation DST-2970 IR administered at 200 mg dose provided increased abiraterone plasma levels, including increased Cmax, and increased AUC₀₋ₜ compared to ZYTIGA® administered at 1,000 mg dose.

Accordingly, subjects administered with the exemplary abiraterone pharmaceutical formulation DST-2970 IR at about one fifth of the dose of ZYTIGA® showed improved absorption and increased abiraterone exposure, which is expected to result in increased therapeutic effect in patients.

When the results are adjusted to take into account the differences in dose between DST-2970 IR administered at 200 mg and ZYTIGA® administered at 1,000 mg, the exemplary formulation DST-2970 IR resulted in more than 6-fold increase in Cmax in fasted human subjects compared to ZYTIGA® administered fasted human subjects.

When the results are adjusted to take into account the differences in dose between DST-2970 IR administered at 200 mg and ZYTIGA® administered at 1,000 mg, the exemplary formulation DST-2970 IR resulted in more than 3-fold increase in AUC₀₋ₜ in fasted human subjects compared to ZYTIGA® administered fasted human subjects.

When administered to fasted human subjects, DST-2970 IR 200 mg provided a 6% decrease in variability of geometric mean of Cₘₐₓ and a 4% decrease in variability of geometric mean of AUC₀₋ₜ as compared to fasted human subjects administered ZYTIGA® at 1,000 mg. In addition, when administered to fed human subjects, DST-2970 IR 200 mg provided a 25% decrease in variability of geometric mean of C. and a 23% decrease in variability of geometric mean of AUC₀ₜ as compared to fasted human subjects administered ZYTIGA® at 1,000 mg.

Furthermore, administration of 200 mg of the DST-2970 IR of the present disclosure to fed human subjects resulted in a negligible or small (e.g., about 30%) decrease in Cmax and negligible or no decrease in AUC₀₋ₜ as compared to administration of 200 mg of DST-2970 IR to fasted human subjects.

### Prophetic Example 17: Clinical Trial in Patients with Metastatic Castration-Resistant Prostate Cancer (mCRPC) and Primary Resistance to abiraterone

This Example describes a prophetic multi-center, open label, dose escalation study to evaluate the safety, tolerability, pharmacokinetics, and anti-prostate specific antigen (PSA) activity of DST-2970 Tablets in metastatic castration-resistant prostate cancer (mCRPC) patients with primary resistance to abiraterone.

The investigational product will be DST-2970 Tablets (a pharmaceutical formulation according to the current disclosure).

The proposed indication will be treatment of mCRPC in patients with primary resistance to abiraterone.

In this study, it will be determined whether DST-2970 Tablets show efficacy in patients who exhibit primary resistance to abiraterone. Patients are defined as having primary resistance if they do not exhibit a prostate specific antigen (PSA) decline after three cycles (1 cycle=4 weeks) of Zytiga® therapy (1,000 mg Abiraterone acetate once daily and 5 mg prednisone twice daily).

For example, a primary objective of this study may include to assess the safety, tolerability, pharmacokinetics, and/or efficacy of DST-2970 tablets in patients with mCRPC and primary resistance to abiraterone therapy, defined as showing no decline in prostate specific antigen (PSA) after three cycles of Zytiga® therapy (1,000 mg Abiraterone acetate once daily and 5 mg prednisone twice daily).

For example, a secondary objective of this study may include, to determine the pharmacokinetic (PK) profile of abiraterone following DST-2970 administration to fasted patients with treatments varying with respect to dose amount and dosing frequency.

The study population will be adult male patients (≥18 years of age) with mCRPC and primary resistance to abiraterone therapy. Patients are defined as having primary resistance if they do not exhibit a prostate specific antigen (PSA) decline after three cycles of Zytiga® therapy (1,000 mg Abiraterone acetate once daily and 5 mg prednisone twice daily).

The study design and duration may be as follows:

This will be a multi-center, open-label, patient study evaluating safety, tolerability, pharmacokinetics, and efficacy of DST-2970 tablets in patients with mCRPC and primary resistance to abiraterone therapy. These are patients showing no decline in prostate specific antigen (PSA) after three cycles of Zytiga® therapy (1,000 mg Abiraterone acetate once daily and 5 mg prednisone twice daily).

Up to 100 patients will be randomized 1:1 into one of two treatment sequences (study arms): Sequence A: once-daily (QD) administration of DST-2970 tablets; and Sequence B three-times daily (TID) administration of DST-2970 tablets. An initial 250 mg dose of DST-2970 Tablets will be administered per the dosing schedule of each treatment group for 1 cycle, at which point each patient's PSA level will be assessed. If a PSA decline of 30% or greater is not achieved per patient, their individual dose will be increased to 500 mg and administered for a second cycle according to the dosing schedule of the treatment group followed by PSA analysis. This iterative dose escalation process will continue for each individual patient until a PSA decline of 30% or greater is achieved or until the maximum tolerable dose is reached.

The dosage forms and route of administration may be as follows:

DST-2970 will be supplied as 50 mg tablets. Patients will receive an initial dose of 250 mg DST-2970 demonstrated in healthy male subjects to produce similar abiraterone exposure (AUC) to the labeled dose of Zytiga® (1,000 mg fasted). The dose frequency of this initial dose will vary according the patients assigned Sequence group. If after the first treatment duration a PSA decline of 30% or greater is not achieved, the dose of DST-2970 will be increased to 500 mg. If after the second treatment duration a PSA decline of 30% or greater is not achieved, the dose of DST-2970 will be increased to 750 mg. This dose escalation will continue until a PSA decline of 30% or greater is achieved or until the maximum tolerable dose is reached.

Regarding safety and tolerability, this study will determine whether DST-2970 Tablets are safe and well tolerated at all evaluated doses and dosing frequencies.

Regarding pharmacokinetics, this study will determine whether DST-2970 Tablets, when administered QD and/or TID, provide abiraterone exposure (AUC) with ascending doses that exceed, or preferably far exceed, the mean exposure limit of Zytiga of 993 ng*hr/ml at steady-state with once-daily fasted administration of 1,000 mg. For example, an increase in AUC, such as AUC₀ₜ, of, or of about, 1.5-fold, 2-fold, 2.5-fold, 3-fold, 3.5-fold, 4-fold, or more, either in individual patients or mean levels in a plurality of patients, may be indicative of positive results. This study will also determine whether DST-2970 Tablets, when administered QD and/or TID, provide peak abiraterone plasma concentrations (Cmax) with ascending doses that exceed, or preferably far exceed, mean of Zytiga® of 226 ng/ml at steady-state with once-daily fasted administration of 1,000 mg. For example, an increase in Cmax of, or of about, 1.5-fold, 2-fold, 2.5-fold, 3-fold, 3.5-fold, 4-fold, 4.5-fold, 5-fold, 5.5-fold, 6-fold, 6.5-fold, 7-fold, or more, either in individual patients or mean levels in a plurality of patients, may be indicative of positive results. This study will also determine whether DST-2970 Tablets, when administered QD and/or TID, provide increased trough abiraterone plasma concentrations (Cₘᵢₙ), such as Cₘᵢₙ of greater than 35 ng/mL.

Regarding efficacy, this study will determine whether administration of DST-2970 Tablets produce PSA declines, such as declines of greater than 30% in a minimum of 20% of patients corresponding to improvements in abiraterone exposure; such as improvements in Cₘₐₓ, AUC, and/or Cₘᵢₙ, relative to the established limits of Zytiga®. For example, if the effect is seen in both the QD and TID groups then, the effect may be indicative of an association with elevated Cmax and/or AUC. The study will determine whether Cmin for the QD group is, or is about, 0 ng/mL. For example, if the effect is only seen in the TID group, then it may be indicative of an association with maintaining an elevated Cₘᵢₙ for the duration of treatment.

### Prophetic Example 18: Clinical Trial in Patients with Metastatic Castration-Resistant Prostate Cancer (mCRPC) and Acquired Resistance to abiraterone

This Example describes a prophetic multi-center, open label, dose escalation study to evaluate the safety, tolerability, pharmacokinetics, and anti-PSA activity of DST-2970 Tablets in metastatic castration-resistant prostate cancer (mCRPC) patients with acquired resistance to abiraterone.

The investigational product will be DST-2970 Tablets (a pharmaceutical formulation according to the current disclosure).

The proposed indication will be treatment of mCRPC in patients with acquired resistance to abiraterone.

In this study, it will be determined whether DST-2970 Tablets show efficacy in patients who exhibit acquired resistance to abiraterone. Patients may be defined as having acquired resistance if they previously showed a PSA decline, such as up to 50%, or more than 50%, compared to pre-treatment levels following treatment for a time (such as at least 12 weeks) with Zytiga® (1,000 mg Abiraterone acetate once daily and 5 mg prednisone twice daily), but who are no longer responding to the therapy as indicated by increasing PSA.

For example, a primary objective of this study may include to assess the safety, tolerability, pharmacokinetics, and/or efficacy of DST-2970 tablets in patients with mCRPC and acquired abiraterone resistance.

For example, a secondary objective of this study may include to determine the pharmacokinetic (PK) profile of abiraterone following DST-2970 administration to fasted patients with treatments varying with respect to dose amount and dosing frequency.

The study population will be adult male patients (≥18 years of age) with mCRPC and acquired resistance to abiraterone therapy.

The study design and duration may be as follows:

This will be a multi-center, open-label, patient study evaluating safety, tolerability, pharmacokinetics, and efficacy of DST-2970 tablets in patients with mCRPC and acquired resistance to abiraterone therapy.

Up to 100 patients will be randomized 1:1 into one of two treatment sequences (study arms): Sequence A: once-daily (QD) administration of DST-2970 tablets; and Sequence B three-times daily (TID) administration of DST-2970 tablets. An initial 250 mg dose of DST-2970 Tablets will be administered per the dosing schedule of each treatment group for 1 cycle (4 weeks), at which point each patient's PSA level will be assessed. If a PSA decline of 30% or greater is not achieved per patient, their individual dose will be increased to 500 mg and administered for a second cycle according the dosing schedule of the treatment group followed by PSA analysis. This iterative dose escalation process will continue for each individual patient until a PSA decline of 30% or greater is achieved or until the maximum tolerable dose is reached.

The dosage forms and route of administration may be as follows:

DST-2970 will be supplied as 50 mg tablets. Patients will receive an initial dose 250 mg of DST-2970 demonstrated in healthy male subjects to produce similar abiraterone exposure (AUC) to the labeled dose of Zytiga® (1,000 mg fasted). The dose frequency of this initial dose will vary according the patients assigned Sequence group. If after the first treatment duration a PSA decline of 30% or greater is not achieved, the dose of DST-2970 will be increased to 500 mg. If after the second treatment duration a PSA decline of 30% or greater is not achieved, the dose of DST-2970 will be increased to 750 mg. This dose escalation will continue until a PSA decline of 30% or greater is achieved or until the maximum tolerable dose is reached.

Regarding safety and tolerability, this study will determine whether DST-2970 Tablets are safe and well tolerated at all evaluated doses.

Regarding pharmacokinetics, this study will determine whether DST-2970 Tablets provide abiraterone exposure (AUC) with ascending doses that exceed, or preferably far exceed, the mean exposure limit of Zytiga of 993 ng*hr/ml at steady-state with once-daily fasted administration of 1,000 mg. For example, an increase in AUC, such as AUC₀₋ₜ, of, or of about, 1.5-fold, 2-fold, 2.5-fold, 3-fold, 3.5-fold, 4-fold, or more, either in individual patients or mean levels in a plurality of patients, may be indicative of positive results. This study will also determine whether DST-2970 Tablets, when administered QD and/or TID, provide peak abiraterone plasma concentrations (Cmax) with ascending doses that exceed, or preferably far exceed, mean of Zytiga® of 226 ng/ml at steady-state with once-daily fasted administration of 1,000 mg. For example, an increase in Cmax of, or of about, 1.5-fold, 2-fold, 2.5-fold, 3-fold, 3.5-fold, 4-fold, 4.5-fold, 5-fold, 5.5-fold, 6-fold, 6.5-fold, 7-fold, or more, either in individual patients or mean levels in a plurality of patients, may be indicative of positive results. This study will also determine whether DST-2970 Tablets, when administered QD and/or TID, provide increased trough abiraterone plasma concentrations (Cₘᵢₙ), such as Cₘᵢₙ of greater than 35 ng/mL.

Regarding efficacy, this study will determine whether administration of DST-2970 Tablets reverse the increasing PSA trend and produce PSA declines, such as declines of greater than 30% in a minimum of 20% of patients corresponding to improvements in abiraterone exposure; i.e., Cₘₐₓ, AUC, and/or Cₘᵢₙ; relative to the established limits of Zytiga®. For example, if the effect is seen in both the QD and TID groups then, the effect may be indicative of an association with elevated Cmax and/or AUC. The study will determine whether Cmin for the QD group is, or is about, 0 ng/mL. For example, if the effect is only seen in the TID group, then it may be indicative of an association with maintaining an elevated Cₘᵢₙ for the duration of treatment.

### Prophetic Example 19: Clinical Trial in Patients with Triple-Negative Breast Cancer (TNBC)

This Example describes a prophetic multi-center, open label, dose escalation study to evaluate the safety, tolerability, and anti-tumor activity of DST-2970 Tablets in patients with triple-negative breast cancer (TNBC).

The investigational product will be DST-2970 Tablets (a pharmaceutical formulation according to the current disclosure).

The proposed indication will be treatment of TNBC.

In this study, it will be determined whether DST-2970 Tablets show efficacy in patients with TNBC. TNBC is cancer that tests negative for estrogen receptors, progesterone receptors, and excess HER2 protein.

For example, a primary objective of this study may include to assess the safety, tolerability, and efficacy of DST-2970 tablets in patients with TNBC. A primary outcome measure will include pathologic complete response (pCR). A secondary outcome measure will include overall survival (OS).

Inclusion criteria for this study may be the following:

Histologically or cytologically confirmed Triple-Negative invasive breast carcinoma; clinical stage IIA-IIIB. Patients will have measurable disease as defined by palpable lesion with caliper and/or a positive mammogram or ultrasound. Bilateral mammogram and clip placement will be required for study entry. Baseline measurements of the indicator lesions will be recorded on the Patient Registration Form. To be valid for baseline, the measurements will have been made within the 14 days if palpable. If not palpable, a mammogram or MRI will be done within 14 days. If palpable, a mammogram or MRI will be done within 2 months prior to study entry. If clinically indicated, x-rays and scans will be done within 28 days of study entry. Patients must have adequate organ function within 2 weeks of study entry, indicated by the following: Absolute neutrophil count>1500/mm3, Hgb>9.0 g/dl and platelet count>100,000/mm3 Total bilirubin<upper limit of normal Creatinine<1.5 mg/dL or calculated cranial cruciate ligament (CrCL)>50 mL/min using the Cockcroft Gault equation serum glutamate oxaloacetate transaminase(SGOT)(AST) or serum glutamic oxaloacetic transaminase (SGPT)(ALT) and Alkaline Phosphatase must be within the range allowing for eligibility. Patients must be over 18 years old. Women of childbearing potential must have a negative serum pregnancy test performed within 7 days prior to the start of treatment. Women of childbearing potential and men must agree to use adequate contraception (barrier method of birth control) prior to study entry and for the duration of study participation.

The study design and duration may be as follows:

This will be a multi-center, open-label, dose escalation study evaluating safety, tolerability, and efficacy of DST-2970 tablets in patients with TNBC. These are patients with breast cancer that tests negative for estrogen receptors, progesterone receptors, and excess HER2 protein.

Up to 100 patients will be randomized 1:1 into one of two treatment sequences (study arms): Sequence A: once-daily (QD) administration of DST-2970 tablets; and Sequence B three-times daily (TID) administration of DST-2970 tablets. An initial 250 mg dose of DST-2970 Tablets will be administered per the dosing schedule of each treatment group for 1 cycle (4 weeks), at which point a biopsy will be performed and pathologic response (PR) will be assessed. If minimal to no tumor response is observed per patient, their individual dose will be increased to 500 mg and administered for a second cycle according the dosing schedule of the treatment group, followed by biopsy and PR assessment. This iterative dose escalation process will continue for each individual patient until response is achieved or until the maximum tolerable dose is reached.

The dosage forms and route of administration may be as follows:

DST-2970 will be supplied as 50 mg tablets. Patients will receive an initial dose 250 mg of DST-2970. The dose frequency of this initial dose will vary according the patients assigned Sequence group. If after the first treatment duration a tumor response is not achieved, the dose of DST-2970 will be increased to 500 mg. If after the second treatment duration response is not achieved, the dose of DST-2970 will be increased to 750 mg. This dose escalation will continue until tumor response is achieved or until the maximum tolerable dose is reached.

Regarding safety and tolerability, this study will determine whether DST-2970 Tablets are safe and well tolerated at all evaluated doses.

Regarding efficacy, this study will determine whether administration of DST-2970 Tablets provides statistically significant improvements in pCR and OS. For example, efficacy may be observed in association with high systemic exposures of abiraterone and consequently highly effective targeting of the androgen receptor (AR) pathway. For example, if the effect is seen in both the QD and TID groups then, the effect may be indicative of an association with elevated Cmax and/or AUC. The study will determine whether Cmin for the QD group is, or is about, 0 ng/mL. For example, if the effect is only seen in the TID group, then it may be indicative of an association with maintaining an elevated Cmin for the duration of treatment.

### Prophetic Example 20: Clinical Trial in Patients with Non-Metastatic Castration-Resistant Prostate Cancer (nmCRPC)

This Example describes a prophetic multicenter, comparative, randomized, double-blind, parallel-group, active-controlled, clinical superiority trial of DST-2970 tablets versus apalutamide in men with non-metastatic castration-resistant prostate cancer (nmCRPC).

The investigational product will be DST-2970 Tablets (a pharmaceutical formulation according to the current disclosure).

The proposed indication will be treatment of nmCRPC.

In this study, it will be determined whether DST-2970 Tablets show superior efficacy over apalutamide in patients with non-metastatic castration-resistant prostate cancer as signified by a delay in the onset of metastasis. Superior efficacy may be associated with more effective targeting of the androgen receptor (AR) pathway via elevated systemic concentrations of abiraterone.

For example, a primary objective of this study may include to evaluate the safety and efficacy of DST-2970 tablets versus apalutamide in adult men with nmCRPC. A primary measure of efficacy will include metastasis-free survival (MFS) by Blinded Independent Central Review (BICR). MFS refers to the time from randomization to the time of first evidence of BICR-confirmed bone or soft tissue distant metastasis or death due to any cause, whichever occurrs first. Radiographic scans (bone scans and computerized tomography [CT] or magnetic resonance imaging [MRI] of the chest, abdomen, and pelvis) will be performed for detection of metastasis throughout the study.

For example, secondary objectives of this study may include the following:

Time to metastasis (TTM), for example defined as the time from randomization to the time of the scan that shows first evidence of BICR-confirmed radiographically detected bone or soft tissue distant metastasis. Radiographic scans (bone scans and CT or MRI of the chest, abdomen, and pelvis) will be performed for detection of metastasis throughout the study.

Progression-free survival (PFS), for example defined as time from randomization to first documentation of BICR-confirmed radiographic progressive disease (PD) (development of distant/local/regional metastasis) or death due to any cause, whichever occurs first. Radiographic scans (bone scans and CT/MRI of chest, abdomen, pelvis) will be performed for detection of metastasis throughout study. PD may be based on Response Evaluation Criteria in Solid Tumors (RECIST) v1.1. In subjects with a measurable lesion, at least 20% increase in sum of diameters of target lesions taking as reference smallest sum on study, and/or an absolute increase of at least 5 mm may be considered as PD. Also, appearance of one or more new lesions may be considered as PD. Also, in subjects with non-measurable disease as per CT/MRI scans, an unequivocal progression/appearance of one or more new lesions may be considered as PD. For new bone lesions detected on bone scans, second imaging (CT/MRI) may be required to confirm PD.

Time to symptomatic progression, for example defined as the time from randomization to documentation of any of the following (whichever occurs earlier): a) development of a skeletal-related event (pathologic fracture, spinal cord compression, or need for surgical intervention or radiation therapy to the bone); b) pain progression or worsening of disease-related symptoms requiring initiation of a new systemic anti-cancer therapy; or c) development of clinically significant symptoms due to loco-regional tumor progression requiring surgical intervention or radiation therapy.

Overall survival, for example defined as the time from randomization to the date of death due to any cause.

Time to initiation of cytotoxic chemotherapy, for example defined as the time from randomization to the date of initiation of cytotoxic chemotherapy for prostate cancer.

Inclusion criteria for this study may be the following:

Histologically or cytologically confirmed adenocarcinoma of the prostate without neuroendocrine differentiation or small cell features with high risk for development of metastases, for example defined as prostate-specific antigen doubling time (PSADT) less than or equal to (<=) 10 months. PSADT may be calculated using at least 3 prostate-specific antigen (PSA) values obtained during continuous ADT (androgen deprivation therapy). Patients will have castration-resistant prostate cancer demonstrated during continuous ADT, for example defined as 3 PSA rises, at least 1 week apart, with the last PSA greater than (>) 2 nanogram per milliliter (ng/mL). Patients will have maintained castrate levels of testosterone within 4 weeks prior to randomization and throughout the study. Patients currently receiving bone loss prevention treatment with bone-sparing agents will be on stable doses for at least 4 weeks prior to randomization. Patients who receive a first-generation anti-androgen (for example, bicalutamide, flutamide, nilutamide) will have at least a 4-week washout prior to randomization and must show continuing disease (PSA) progression (an increase in PSA) after washout. At least 4 weeks will have elapsed from the use of 5-alpha reductase inhibitors, estrogens, and any other anti-cancer therapy prior to randomization. At least 4 weeks will have elapsed from major surgery or radiation therapy prior to randomization. Patients will show resolution of all acute toxic effects of prior therapy or surgical procedure to Grade<=1 or baseline prior to randomization. Patients will show adequate organ function according to protocol-defined criteria. Administration of growth factors or blood transfusions may not be allowed within 4 weeks of the hematology labs required to confirm eligibility.

The study design and duration may be as follows:

This will be a multicenter, comparative, randomized, double-blind, parallel-group, active-controlled, clinical superiority trial of DST-2970 tablets versus apalutamide in men with nmCRPC.

Patients will be randomized 1:1 into one of two treatment sequences (study arms): Sequence A: once-daily (QD) administration of 240 mg of apalutamide tablets; and Sequence B: QD administration of 400 mg of DST-2970 tablets. Patients will be evaluated periodically according to the aforementioned measures for indications of metastasis. Statistically significant improvement in metastasis free survival will indicate superiority of DST-2970 over apalutamide.

The dosage forms and route of administration may be as follows:

Apalutamide will be supplied as 60 mg tablets. DST-2970 will be supplied as 50 mg tablets. Tablets will be administered orally per the Sequence dosing schedule.

Regarding safety and tolerability, this study will determine whether DST-2970 Tablets are safe and well tolerated at all evaluated doses.

Regarding efficacy, this study will determine whether administration of DST-2970 Tablets provides statistically significant improvements versus apalutamide in the primary and secondary measures. For example, efficacy may be observed in association with high systemic exposures of abiraterone and consequently highly effective targeting of the androgen receptor (AR) pathway.

### Prophetic Example 21: Pharmaceutical Formulations of the Present Disclosure having abiraterone in its Active Form, or abiraterone in a Modified Form, such as a Pharmaceutically Acceptable Salt, Ester, Derivative, Analog, Prodrug, Hydrate, or Solvate Thereof

It is contemplated that the exemplary formulations described in the Examples herein having abiraterone or abiraterone acetate will show the same or similar results in terms of pharmacokinetics and therapeutic effects. Accordingly, it is contemplated that active abiraterone and abiraterone and in a modified form, such as a pharmaceutically acceptable salt, ester, derivative, analog, prodrug, hydrate, or solvate thereof will show the same or similar results in terms of pharmacokinetics and therapeutic effects.

### Example 22: Materials and Methods for Examples 23 to 28

The following Materials were used to generate the results described in Examples 23 to 28:

Materials. Abiraterone active pharmaceutical ingredient (API) was purchased from Attix Pharma (Ontario, Cannada). Hydroxy propyl β cyclodextrin, i.e., Kleptose® HPB was purchased from Roquette America (USA). Microcrystalline cellulose, i.e., Avicel PH-102 was purchased from FMC Corporation (Pennsylvania, USA). Mannitol, i.e., Pearlitol 200SD was purchased from Roquette America (USA). Crosslinked sodium carboxy methyl cellulose, i.e., Vivasol® was purchased from JRS Pharma (New York, USA). Hypromellose acetate succinate HMP grade, i.e., AQOAT® was purchased from Shin-Etsu (New Jersey, USA). Colloidal Silicon Dioxide, i.e., Aerosil® 200 P was purchased from Evonik Industries (New Jersey, USA). Magnesium Stearate was purchased from Peter Greven (Muenstereifel, Germany) The fasted state simulated intestinal fluid (FaSSIF) dissolution media was prepared using FaSSIF/FeSSIF/FaSSGF powder purchased from Biorelevant.com (Surrey, UK). Abiraterone acetate tablets, i.e., Zytiga® (250 mg abiraterone acetate) were purchased from a pharmacy, they were manufactured for Janssen Biotech (Pennsylvania, USA). The solvents used for HPLC analysis were of HPLC grade. All other chemicals and reagents used for dissolution and HPLC analysis were of ACS grade.

The following Methods were used to generate the results described in Examples 23 to 28:

KinetiSol® Processing. Abiraterone KinetiSol® Solid Dispersions (KSDs) with different drug loading were prepared using KinetiSol® technology (DisperSol Technologies LLC, Texas). Initially, all KSDs were prepared using research-scale compounder (“Formulator”) designed and manufactured by DisperSol Technologies LLC (Texas, USA). Later, amorphous KSDs were prepared using manufacturing-scale compounder (“Manufacturing compounder”) designed and manufactured by DisperSol Technologies LLC (Texas, USA). Prior to compounding, the drug abiraterone and the oligomer HPBCD (table 3.1) were accurately weighed, and thoroughly mixed to prepare physical mixtures (PM). The physical mixtures were charged into the KinetiSol® compounder chamber. Inside the chamber, a shaft with protruding blades was rotated at varying incremental speeds ranging from 500 rpm to 7000 rpm, without external heat addition, to impart frictional and shear forces to the sample material. The temperature of the mass was monitored using an infrared probe. When molten mass temperature reached a value of 150-180° C., the mass was rapidly ejected, collected, and pressed between two stainless steel plates to rapidly quench the sample.

Milling The quenched mass obtained after KinetiSol® processing was milled in small batches, using a lab scale rotor mill, i.e., IKA tube mill 100 (IKA Works GmbH & Co. KG, Staufen, Germany). For milling, the fragments of quenched mass were loaded into a 20 mL grinding chamber which was operated between 10000-20000 rpm grinding speed for 60 seconds. The milled material was subsequently passed through a #60 mesh screen (≤250 μm). Material retained above the screen, i.e., >250 μm was cycled through the mill with the same parameters and this process of milling and sieving was repeated until all material passed through the screen. The resultant material<250 μm was labeled as KSD.

Melt-quenching Abiraterone. In order to provide neat amorphous abiraterone reference sample for nuclear magnetic resonance spectroscopy and Raman spectroscopy, abiraterone was melt-quenched. A small quantity of abiraterone (<0.5 grams) was added to an open scintillation vial, and blow torched for a few seconds until entire quantity of abiraterone melted. The scintillation vial containing the molten mass was immediately submerged into liquid nitrogen. When the intensity of nitrogen boiling subsided, the vial was transferred into a vacuum desiccator and vacuum was applied for about 2 hrs. After 2 hrs, the vacuum was released and the vial was removed from the desiccator. The quenched abiraterone mass was scrapped from the vial, lightly grounded using a mortar pestle and sieved via a #60 mesh screen (≤250 μm). The melt quenched abiraterone was placed in the freezer until further use.

Modulated Differential Scanning calorimetry. Thermal analysis was conducted by modulated differential scanning calorimetry (mDSC) using differential scanning calorimeter model Q20 (TA Instruments, Delaware, USA) equipped with a refrigerated-based cooling system and an autosampler. The API and KSD samples were prepared by weighing 5-10 mg of the material and loading it into a Tzero pan. The pan was sealed with Tzero lid using a Tzero press. Following the sample equilibration at 30° C. for 5 min, the temperature was ramped at 5° C./min up to 250° C. with modulation of ±1° C. every 60 seconds. Nitrogen was used as the sample purge gas at a flow rate of 50 mL/min. The data was collected using TA Instrument Explorer software (TA Instruments, Delaware, USA) and processed using Universal Analysis software (TA Instruments, Delaware, USA).

HPLC Analysis. A stability indicating high-performance liquid chromatography (HPLC) method was developed for chemical analysis of abiraterone KSDs. A Dionex Ultimate 3000 HPLC system (ThermoFisher Scientific, Massachusetts, USA) was used for reverse phase HPLC analysis. The HPLC column was a Kinetex® XB C18, 150 mm×4.6 mm, 2.6 μm (Phenomenex, California, USA). Mobile phase A was 20 mM ammonium formate buffer (pH 3) and mobile phase B was degassed acetonitrile. A gradient profile with higher aqueous phase initially, followed by gradual increase in organic phase was designed. The flow rate was 0.9 mL/min and the run time was 42 min. The column was held at 35° C., and the data was collected at a single wavelength of 254 nm. Samples were prepared at a nominal concentration of 0.5 mg/mL level with 7:2:1 methanol:isopropyl alcohol:tetrahydrofuran as the standard/sample diluent. All samples were filtered through 0.45 μm PVDF syringe filters (GE Healthcare Life-Sciences, Pennsylvania, USA), prior to analysis. Samples chromatography was analyzed using Chromeleon™ software, version 7.0 (ThermoFisher Scientific, Massachusetts, USA).

Solid state Nuclear Magnetic Resonance Spectroscopy. The one-dimensional (1D) ¹³C solid state Nuclear Magnetic Resonance Spectroscopy (ssNMR), was conducted at NMR Lab, University of Texas at Austin (Texas, USA). The ¹³C ssNMR spectra were collected using Bruker AVANCE™ III HD 400 MHz instrument (Bruker Corporation, Massachusetts, USA). A cross polarization experiment was conducted using 4 mm MAS probe and the ¹³C frequency employed was 100.62 MHz. The contact time was set to 2 ms, spin rate was set to 10 KHz and the relaxation delay ranged from 2 to 30 seconds. The temperature was set to 300.0 K. The chemical shift reference standard adamantane 38.48 ppm was used. The data was collected using Bruker NMR software (Bruker Corporation, Massachusetts, USA). The data was processed using MNOVA software, version 14 (Santiago de Compostela, Spain).

Two-dimensional (2D) ¹³C-¹H heteronuclear correlation (HETCOR) spectra were acquired using a Bruker AVANCE III HD 400 triple-resonance spectrometer operating at ¹H frequency of 400.13 MHz in the Biopharmaceutical NMR Laboratory (BNL) of Preclinical Development at Merck Research Laboratories (Merck & Co., Inc. West Point, Pa.). Experimental temperature at 298 K and a MAS frequency of 12 kHz were utilized. All data were processed in Bruker TopSpin software. The ¹³C-¹H HETCOR experiments were carried out using a CP contact time of 2 ms and a recycle delay of 2 seconds.

Raman Spectroscopy. Raman spectroscopy was conducted using HyperFlux™ PRO Plus (HFPP) Raman spectroscopy system (Tornado Spectral Systems, Ontario, Canada). The

API, PM, KSDs and melt-quench abiraterone samples were loaded on an aluminum stage. The samples were subjected to a laser beam with a wavelength of 785 nm and power of 200 mW. 50 exposures were collected per spectrum and 3 spectra were collected per sample. Exposure time of 100 ms was employed. Cosmic ray removal and dark spectral correction were enabled. The spectral data was collected using SpectralSoft software (Tornado Spectral Systems, Ontario, Canada). The spectral data pre-processing and multivariate analysis was done using Unscrambler X software (Camo Analytics, Oslo, Norway).

Phase Solubility Analysis. Phase solubility analysis was conducted in two separate media, i.e., 0.01N HCl (pH 2.0) and FaSSIF (prepared in 50 mMol Phosphate Buffer pH 6.8). Solutions of HPBCD ranging from 0 mg/mL to 600 mg/mL were prepared in each media in scintillation vials. An excess of abiraterone was added to each vial and the vials were sonicated for 30 minutes and placed on a bench. Samples were pulled from each vial at time points of 48 hrs and 7 days. The samples were centrifuged using an ultracentrifuge (Eppendrof, Hamburg, Germany) The supernatants were further diluted using the HPLC diluent and analyzed by HPLC method mentioned above to find the concentration of abiraterone.

Stability Analysis. Stability analysis was performed at elevated temperatures. KSD samples were loaded into a scintillation vial and heated on a hot plate set at 90° C., for 6 hrs. The samples were then analyzed by XRPD as stated above. Upon XRPD analysis, the samples were re-heated on a hot plate set at 150° C., for 6 hrs and the samples were re-analyzed by XRPD.

In vitro Dissolution Study. An in vitro non-sink, gastric transfer dissolution method was developed to analyze the dissolution of abiraterone API and KSDs. For dissolution analysis samples equivalent to 44.6 mg of abiraterone API, were loaded in an Erlenmeyer flask (dissolution vessel) containing 50 mL of 0.01N HCl (pH 2.0), placed in an incubator-shaker-Excella E24 (New Brunswick Scientific, New Jersey, USA) set to 37° C. and a rotational speed of 180 rpm. After 30 min, 50 mL of FaSSIF (prepared in 50 mMol Phosphate Buffer pH 6.8) was added to the dissolution vessel. At pre-determined time points, samples were drawn from the dissolution vessel and centrifuged using an ultracentrifuge (Eppendrof, Hamburg, Germany) The supernatants were further diluted using the HPLC diluent and analyzed by HPLC method mentioned above. The area under drug dissolution curve (AUDC) was calculated by the linear trapezoidal method.

Tableting. The KSD and tableting excipients Avicel PH-102, Pearlitol 200SD, Vivasol®, Kleptose®, AQOAT®, Aerosil® 200 P and magnesium stearate were accurately weighed and dispensed. Aerosil® 200 P, was sieved through #40 (420 μm) until, all material passed through the sieve. The KSD and all tableting excipients except magnesium stearate were loaded in a vial and mixed using a vortex mixer (Thermo Scientific, Massachusetts, USA). Magnesium stearate was then added to the vial and blended using a spatula. The resultant tableting blend was then dispensed in aliquots equivalent to 50 mg of abiraterone. Each aliquot was loaded in the tablet die and compressed using a single station hand tablet press (BVA Hydraulics, Missouri, USA), with target hardness of 8-12 kP.

In Vivo Pharmacokinetic Study in Beagle Dogs. An in vivo pharmacokinetic study in fasted non-naïve male beagle dogs was carried out at Pharamaron (Ningbo, China). The animal study was conducted according to an approved Pharmaron IACUC protocol #PK-D-06012018. The 50.0 mg equivalent abiraterone tablets along with a 250 mg equivalent abiraterone acetate tablet-Zytiga® were analyzed. Each study arm for each formulation, consisted of 3 dogs. The dogs were fasted overnight prior to dosing and the food was returned after 4 hours post dosing. Each dog was administered a single tablet of respective formulation (as per the study arm), along with post dose flush of 40 mL sterile water. At pre-defined timepoints of 0.25, 0.5, 1, 1.5, 2, 3, 4, 6, 8, 10, 12, 16, 18, 24, 36 and 48 hrs post-dose, 1 mL blood samples were drawn from each dog by venipuncture of a peripheral vessel and placed in into tubes containing sodium heparin anticoagulant. The blood samples were centrifuged to isolate the plasma. The plasma samples were analyzed using liquid chromatography with tandem mass spectrometry (LC-MS/MS) for abiraterone content.

Pharmacokinetic Analysis. Pharmacokinetic parameters were estimated using PhoenixTM WinNonlin software, version 6.1 (Certara, New Jersey, USA) using a non-compartmental approach consistent with the oral route of administration. The area under the plasma concentration-time curve (AUC) was calculated by the linear trapezoidal method. The relative bioavailability, i.e., F value was calculated using the following formula:

$$F = \frac{\begin{matrix}{AUC}_{( 0 ‐ 48 hr ) ( test abiraterone tablet )} * \\ {Dose}_{( abiraterone dose ) ( reference abiraterone acetate tablet )}\end{matrix}}{\begin{matrix}{AUC}_{( 0 ‐ 48 hr ) ( reference abiraterone acetate tablet )} * \\ {Dose}_{( abiraterone dose ) ( test abiraterone tablet )}\end{matrix}}$$

### Example 23: Preparation of KinetiSol® Solid Dispersions (KSDs)

Table 19 lists the KSDs composition and processing parameters. Lots 1 to 5 PMs were all processible by KinetiSol® technology. Initially, all lots were processed on a research scale compounder. Later, in order to yield greater amount of material for further testing, Lots 1 to 3 KSDs were processed on a manufacturing scale compounder. The total processing time for Lots 1 to 4 KSDs was less than 20 seconds, whereas that for Lot 5 KSD was 41.5 seconds. Considerable amount of material sticking was observed for Lots 4 and 5 KSDs. All lots were easily milled and sieved via #60, to yield KSD powders having particle size of <250 μm.

TABLE 19
KSDs composition and processing parameters

| | | | | | | Shear | |
|---|---|---|---|---|---|---|---|
| | | | | | | Stress | Total |
| | Composition | | | | Processing | (Rotational | Processing |
| Lot | Drug | Oligomer | KinetiSol ® | Batch | Temperature | speed- | Time |
| No. | (% wt) | (% wt) | Compounder | Size (g) | (° C.) | rpm) | (secs) |
| 1 | Abiraterone (10) | HPBCD | Manufacturing | 90 | 150 | 2400, 2700 | 16 |
| | | (90) | Compounder | | | | |
| 2 | Abiraterone (20) | HPBCD | Manufacturing | 90 | 180 | 2400, 2700 | 18 |
| | | (80) | Compounder | | | | |
| 3 | Abiraterone (30) | HPBCD | Manufacturing | 90 | 180 | 500, 2700, | 16.5 |
| | | (70) | Compounder | | | 3000 | |
| 4 | Abiraterone (40) | HPBCD | Formulator | 10 | 160 | 1000, 6000 | 19.3 |
| | | (60) | | | | | |
| 5 | Abiraterone (50) | HPBCD | Formulator | 10 | 160 | 1000, 6000, | 41.5 |
| | | (50) | | | | 7000 | |

Conventionally, drug-CD pharmaceutical compositions have been prepared by solvent (e.g. organic solvents, carbon dioxide) based technologies such as spray drying, freeze drying, solvent evaporation, kneading using a solvent and supercritical fluid process (Modekar and Patil 2016; Jug, Becirevic-Lacan, and Bengez 2009; Gala 2015; Jug and Mura 2018; Semcheddine et al. 2015; Li et al. 2018). There are several disadvantages associated with these technologies such as limited scalability, high consumption of energy, use of toxic organic solvents, challenging solvent removal and potential of drug degradation (Jug and Mura 2018). Certain solvent free technologies such as microwave irradiation, sealed heating and hot melt extrusion have also been reported for preparation drug-CD pharmaceutical compositions (Thiry et al. 2017; Wen et al. 2004; Mura et al. 1999). The major drawback of these technologies is drug degradation due to microwave irradiation or heating (Jug and Mura 2018). Grinding is another solvent free method that has been used to prepare drug-CD pharmaceutical compositions (Ramos et al. 2013; Borba et al. 2015). Typical grinding time for drug-CD ranges in order of several minutes to hours (Jug and Mura 2018). Subjecting drugs to high physical stress for longer duration of time, which is typical of grinding process, can lead to significant drug degradation (Savjani, Gajjar, and Savjani 2012). KinetiSol® is a thermokinetic, solvent free technology, that does not require application of external heat and its typical processing times are in order of seconds usually <30 seconds (Ellenberger, Miller, and Williams 2018). Thus KinetiSol® is a promising technology for processing drug-CD pharmaceutical compositions.

Using KinetiSol® technology, abiraterone-HPBCD KSDs with 10% to 50% drug loading were first developed on research scale compounder (i.e. formulator). The Formulator is suitable for formulation screening and feasibility studies on small scale, since it can process up to 15 g of material per batch (Ellenberger, Miller, and Williams 2018). Whereas the manufacturing scale compounder, also known as manufacturing compounder, is suitable for making preclinical and early stage clinical supplies since it can process up to 350 g of material per batch (Ellenberger, Miller, and Williams 2018). Since, Lots 1 to 3 KSDs were to be tableted for animal studies, these lots were made again on manufacturing compounder to yield higher KSD quantity. The processing parameters of Lots 1 to 3 PMs were easily transferred from formulator to manufacturing compounder using scaling factors, thereby demonstrating that KinetiSol® is a scalable technology. It was seen that 10% to 40% drug loadings (i.e. Lots 1 to 4 KSDs) required less total processing time (<20 seconds) to reach the target temperature as compared to that for 50% drug loaded Lot 5 KSD (41.5 seconds) (Table 19). This could be because higher amount of shear stress for longer duration is needed to thermokinetically process high melting abiraterone, present in larger quantity. The processing temperatures for all 5 lots were comparable and ranged from 150-180° C. (Table 19). For Lots 3 to 5 KSDs a lower rotational speed was adopted initially, i.e., 500 rpm, 1000 rpm and 1000 rpm respectively and a faster higher rotational speed was adopted thereafter, i.e., 2700 rpm, 6000 rpm and 6000 rpm respectively, to assure uniform mixing of drug since they contained higher drug loading (Table 19). Lots 4 and 5 KSDs contained higher amount of molten abiraterone leading to material sticking. This issue can be easily solved by addition of lubricant such as sodium stearyl fumarate.

Overall, KSDs with drug loadings of 10% to 50% were processible by KinetiSol® technology and were easily, quenched, milled and sieved to yield KSD powders.

### Example 24: Physico-Chemical Analysis of KSDs

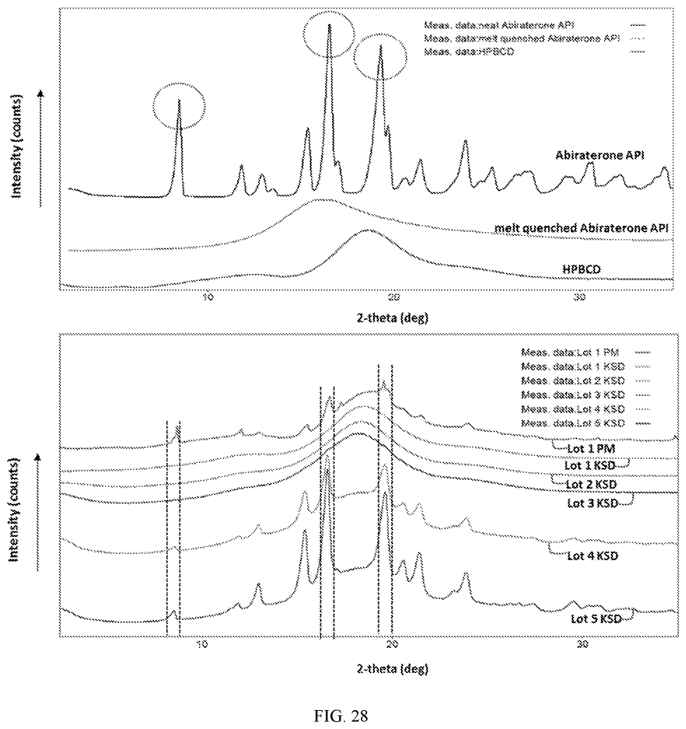

FIG. 28 illustrates X-ray diffractograms of neat abiraterone API, melt quenched abiraterone API, HPBCD, Lot 1 PM and Lots 1 to 5 KSDs. The X-ray diffractogram of neat abiraterone API, showed sharp diffraction peaks. It showed characteristic peaks at 8.43° , 16.54° and 19.31°. The melt quenched abiraterone API X-ray diffractogram showed no sharp diffraction peaks and displayed a halo pattern. X-ray diffractogram of HPBCD displayed a halo pattern as well. The X-ray diffractogram of Lot 1 PM showed sharp diffraction peaks. It displayed sharp peaks at 8.70°, 16.72° and 19.55°, corresponding to characteristic peaks of neat abiraterone API. Additionally, Lot 1 PM displayed peaks at 12.08°, 15.51°, 17.29°, 21.48° and 23.98°, slightly differing in positions, to the peaks observed in neat abiraterone API diffractogram. X-ray diffractograms of Lots 1 to 3 KSDs showed a complete halo pattern and no diffraction peaks. X-ray diffractograms of Lot 4 and 5 KSDs showed sharp diffraction peaks at 8.52°, 16.54° and 19.60°, corresponding to characteristic peaks of neat abiraterone API. Additionally, Lot 4 and 5 KSDs displayed certain smaller diffraction peaks, which were also observed in neat abiraterone API diffractogram.

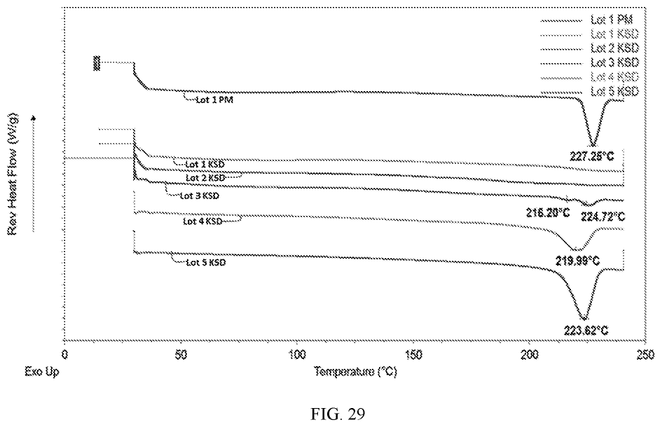

The mDSC thermograms of Lot 1 PM and Lots 1 to 5 KSDs are illustrated in FIG. 29. Lot 1 PM showed a sharp melting endotherm at 227.25° C. Lots 2 and 3 KSDs showed no significant thermal events at evaluated mDSC run conditions. Lot 3 KSD showed a small thermal event at 216.20° C. and a melting endotherm at 224.72° C. Lot 4 KSD showed a broad melting endotherm at 219.99° C. Similarly, Lot 5 KSD showed a melting endotherm at 223.62° C.

The HPLC analysis showed that Lot 1 to 3 KSD had total impurities of 0.28%, 0.37% and 0.38% respectively. None of these lots had an individual unknown impurity of ≥0.2%.

The X-ray diffractogram (FIG. 28) of neat abiraterone API, showed sharp diffraction peaks, thereby indicating its crystalline nature. The melt-quenched abiraterone API showed halo XRPD pattern (FIG. 28), thus suggesting that abiraterone was converted into neat amorphous form. It is well known that neat amorphous APIs are highly unstable and have a strong tendency to recrystallize (Knapik-Kowalczuk et al. 2019). Thus, in order to prevent recrystallization, melt quench abiraterone API was stored in freezer below 0° F. The X-ray diffractogram (FIG. 28) of HPBCD showed a halo pattern similar to that reported by Gala and Ren et al., thereby indicating that HPBCD was amorphous in state (Gala 2015; Ren et al. 2009). The native CDs are crystalline in nature, due to their intermolecular hydrogen bonding (Gala 2015; Loftsson et al. 2005). However, due to alkyl substitution, their intermolecular hydrogen bonding is disrupted leading to amorphous nature of modified CDs such as HPBCD (Sharma and Baldi 2016; Loftsson et al. 2005). The Lot 1 PM displayed sharp diffraction peaks (FIG. 28) thus indicating its crystalline nature. The slight difference in XRPD peak positions of Lot 1 PM compared to neat abiraterone API are due to the contribution of amorphous halo pattern of HPBCD in Lot 1 PM. Lots 1 to 3 KSDs showed a halo XRPD pattern (FIG. 28), thus indicating that abiraterone API was converted into amorphous form in these KSDs. However, sharp diffraction peaks observed in X-ray diffractograms (FIG. 28) of Lots 4 and 5 KSDs, indicated that abiraterone API in these KSDs was mostly in crystalline form. Also, since the diffraction peaks observed for Lots 4 and 5 KSDs largely corresponded to the peaks of neat abiraterone API, it can be inferred that KinetSol® technology did not change the polymorphic form of abiraterone API in these KSDs.

The melting peak observed in mDSC thermogram (FIG. 28) of Lot 1 PM corresponded to the melting point of abiraterone around 228° C. (Solymosi et al. 2018). Since, no melting endotherms corresponding to abiraterone API were observed in mDSC thermograms of Lots 2 and 3 KSDs, it further substantiates that abiraterone API was converted to amorphous form in these lots. Interestingly, the two small thermal events observed in mDSC thermogram (FIG. 29) of Lot 3 KSD indicates that some amount of abiraterone in Lot 3 KSD is still in crystalline form. This however conflicts with XRPD pattern of Lot 3 KSD. Thus, we analyzed the non-reversible heat flow v/s temperature profile (data not shown) of Lot 3 KSD mDSC thermogram and saw two broad recrystallization endotherms prior to the melting endotherms observed in reversible heat flow v/s temperature profile, which shows that the melting events were largely due to mDSC run parameters. The broad melting endotherms seen in mDSC thermogram (FIG. 29), of Lots 4 and 5 KSDs indicates the melting of crystalline abiraterone API and substantiates the observations in their XRPD diffractograms (FIG. 28). It should be noted the melting endotherms observed for Lots 4 and 5 KSDs were at lower temperature than the melting point of abiraterone. This could be because of the eutectic phenomena due to interaction between abiraterone-HPBCD resulting from intimate abiraterone-HPBCD mixing and thermokinetic processing by KinetiSol® technology (Gala, Pham, and Chauhan 2013).

Since, the total percent purity of abiraterone in all the KSDs was >99.50%, it can be concluded that abiraterone did not degrade during KinetiSol® processing.

Hence overall, KinetiSol® technology was able to render KSDs with drug loading of 10% to 30%, to physically amorphous and chemically stable form.

### Example 25: Solid State Interaction Between Abiraterone and HPBCD within KSDs

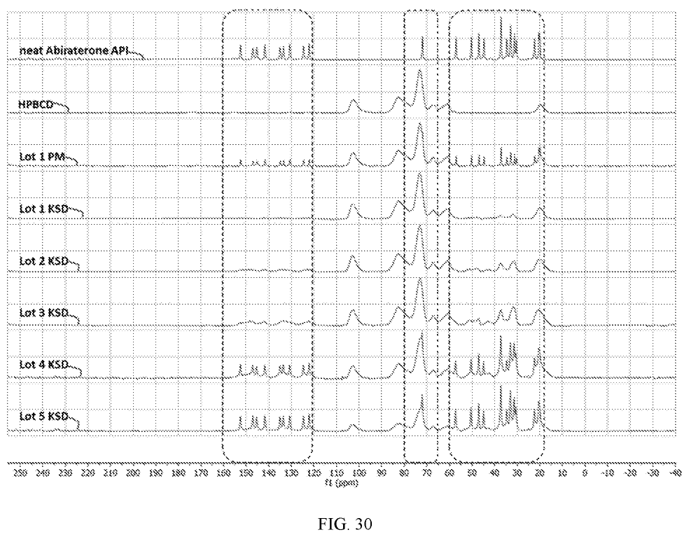

FIG. 30 illustrates ¹³C ssNMR spectra of neat abiraterone API, HPBCD, Lot 1 PM and Lots 1 to 5 KSDs. 1D ¹³C ssNMR spectrum of neat abiraterone API, showed 21 sharp signals. It showed 11 signals between 20 to 60 ppm, a signal at about 72 ppm and 9 signals between 120 to 160 ppm. HPBCD showed 6 broad signals at about 19 ppm, 61 ppm, 67 ppm, 73 ppm, 82 ppm and 102 ppm. Lot 1 PM showed a mix of sharp and broad signals corresponding to both neat abiraterone API and HPBCD. Lot 1 to 3 KSDs showed major signals corresponding to HPBCD. They showed some broad signals between 20 to 60 ppm corresponding to neat abiraterone API, but the signals between 120 to 160 ppm corresponding to neat abiraterone API were absent or extremely broadened in Lot 1 to 3 KSDs ¹³C ssNMR spectra. Lot 4 and 5 KSDs showed a mix of sharp and broad signals corresponding to both neat abiraterone API and HPBCD.

The raw Raman spectra of melt quenched abiraterone API and KSDs showed interference due to fluorescence. Hence, spectral preprocessing was done by converting the raw spectra into second derivative using a second-degree polynomial and a Savitsky-Golay 31-point smoothing filter. The second derivative spectra were then scatter-corrected by application of a standard normal variate transformation. Table 20 lists Raman peak positions for neat abiraterone API, melt quenched abiraterone API, Lot 1 PM and Lots 1 to 5 KSDs. Table 21 lists Raman peak shifts for melt quenched abiraterone API, Lot 1 PM and Lots 1 to 5 KSDs. Specifically, Raman peak positions corresponding to pyridine ring vibrations, steroid moiety vibrations, C=C vibrations in B and D ring of abiraterone are listed. For melt quenched API, the peak shifts with respect to neat abiraterone API are listed. For Lot 1 PM and Lots 4 and 5 KSDs peak shifts with respect to neat abiraterone API are calculated, since they all majorly contain abiraterone in crystalline form. For Lots 1 to 3 KSDs peak shifts with respect to melt quenched abiraterone API are calculated, since they all majorly contain abiraterone in amorphous form.

TABLE 20
Raman peak positions for neat abiraterone API, melt quenched
abiraterone API, Lot 1 PM and Lots 1 to 5 KSDs.

| | | | | | | | | |
|---|---|---|---|---|---|---|---|---|
| | | melt | | | | | | |
| | neat | quenched | | | | | | |
| | Abiraterone | Abiraterone | Lot 1 | Lot 1 | Lot 2 | Lot 3 | Lot 4 | Lot 5 |
| Theoretical | API | API | PM | KSD | KSD | KSD | KSD | KSD |
| assignments | (a) | (b) | (c) | (d) | (e) | (f) | (g) | (h) |

Peak Positions [Wavenumber (cm^{- 1})]

| | | | | | | | | |
|---|---|---|---|---|---|---|---|---|
| Pyridine | 1024 | 1025 | 1024 | 1028 | 1027 | 1027 | 1025 | 1025 |
| ring and | | | | | | | | |
| steroid | | | | | | | | |
| moiety | | | | | | | | |
| vibrations | | | | | | | | |
| Whole | 1049 | 1048 | 1049 | 1050 | 1050 | 1050 | 1049 | 1049 |
| abiraterone | | | | | | | | |
| molecule | | | | | | | | |
| vibrations | | | | | | | | |
| C = C | | 1584 | | 1586 | 1586 | 1586 | | |
| (D-ring) + | 1593 | 1599 | 1594 | 1603 | 1602 | 1601 | 1594 | 1594 |
| pyridine | | | | | | | | |
| C = C (B-ring | 1663 | 1667 | 1663 | 1671 | 1669 | 1668 | 1665 | 1665 |

TABLE 21
Raman peak shifts for melt quenched abiraterone
API, Lot 1 PM and Lots 1 to 5 KSDs.

| | | | | | | | |
|---|---|---|---|---|---|---|---|
| | melt | | | | | | |
| | quenched | Lot 1 | Lot 1 | Lot 2 | Lot 3 | Lot 4 | Lot 5 |
| Theoretical | Abiraterone API | PM | KSD | KSD | KSD | KSD | KSD |
| assignments | (b-a) | (c-a) | (d-b) | (e-b) | (f-b) | (g-a) | (h-a) |

Peak Shifts [Wavenumber (cm−1)]

| | | | | | | | |
|---|---|---|---|---|---|---|---|
| Pyridine ring and | 1 | 0 | 3 | 2 | 2 | 1 | 1 |
| steroid moiety | | | | | | | |
| vibrations | | | | | | | |
| Whole abiraterone | −1 | 0 | 2 | 2 | 2 | 0 | 0 |
| molecule | | | | | | | |
| vibrations | | | | | | | |
| C = C | | | 2 | 2 | 2 | | |
| (D-ring) + | 6 | 1 | 4 | 3 | 2 | 1 | 1 |
| pyridine | | | | | | | |
| C = C (B-ring) | 4 | 0 | 4 | 2 | 1 | 2 | 2 |

The 1D ¹³C ssNMR spectra (FIG. 30) of neat abiraterone API, Lot 1 PM, Lots 4 and 5 KSDs showed major sharp signals thereby indicating their crystalline nature. Whereas, the ¹³C ssNMR spectra (FIG. 30) of HPBCD and Lots 1 to 3 KSDs showed major broad signals thereby indicating their amorphous nature. This further validates our inferences from X-ray diffractometry and modulated differential scanning calorimetry. In order to understand solid state interactions within KSDs we first assigned the ¹³C ssNMR signals to carbon atoms in abiraterone and HPBCD. In ¹³C ssNMR spectrum of neat abiraterone API, the signals from 20 ppm to 60 ppm can be assigned to sp³ hybridized carbons of abiraterone, which are C1, C2, C4, C6, C8, C9, C10, C11, C12, C13, C14, C15, C23 and C24; the signal at about 72 ppm can be assigned to C3 and signals between 120 ppm to 160 ppm can be assigned to sp² hybridized carbons of abiraterone, which are C5, C7, C16, C17, C18, C19, C20, C21 and C22. In ¹³C ssNMR spectrum of HPBCD the signal assignments are 19 ppm (hydroxypropyl group); 61 ppm (C6); 67 ppm (hydroxypropyl group); 73 ppm (C2, C3, C5); 82 ppm (C4) and 102 ppm (C1) of glupyranose unit in HPBCD. Similar HPBCD ¹³C ssNMR spectrum has been reported in literature (Pessine, Calderini, and Alexandrino 2012). Since, ¹³C ssNMR spectrum of Lot 1 PM showed additive signals from both neat abiraterone API and HPBCD, it can be inferred that there is no interaction between abiraterone and HPBCD in the PM. The absence of abiraterone sp² hybridized carbons' signals between 120 ppm to 160 ppm in Lots 1 KSDs ¹³C ssNMR spectrum, indicates that the B-ring, D-ring and pyridine ring of abiraterone are interacting with HPBCD, and likely covered/ included within HPBCD cavity. For Lots 2 and 3 KSDs, these signals have broadened thus indicating some interaction between B-ring, D-ring and pyridine ring of abiraterone with HPBCD, but unlike Lot 1 KSD, not all the amorphous abiraterone in these lots is interacting with HPBCD. The absence of abiraterone C3 signal and broadening of abiraterone sp³ hybridized carbon signals between 20 ppm to 60 ppm, in ¹³C ssNMR spectra of Lots 1 to 3 KSDs suggests interaction between A-ring, C-ring of abiraterone with HPBCD in these lots. Since, ¹³C ssNMR spectra of Lots 4 and 5 KSDs show peaks corresponding to both abiraterone and HPBCD with minimal broadening, thus it can be concluded that there is minimal to no interaction between abiraterone and HPBCD in these KSD lots.

The peak assignments (Table 20) for Raman spectrum of neat abiraterone API was done based on Raman characteristic group frequencies reported in literature (Long 2004; Stolarczyk et al. 2018). The peak shifts (Table 21) for melt quenched abiraterone API are pronounced due to hydrogen bonding disruption in amorphous abiraterone, thereby changing its chemical environment. No major peak shifts are observed for Lot 1 PM, thus reconfirming no interaction between abiraterone and HPBCD in the PM. Amongst KSDs, highest peak shifts are observed for Lot 1 KSD thus suggesting maximum interaction between abiraterone and HPBCD in Lot 1 KSD. The region between 1535-1700 cm⁻¹, has peaks related only to abiraterone API and there is no interference due to HPBCD, hence we focus on that region. The peak shifts related to C=C vibrations in B-ring, D-ring and pyridine ring of abiraterone are of high magnitude in Lot 1 KSD thus suggesting that these rings have interacted with HPBCD and are likely included in its hydrophobic pocket. These peak shifts reduce in magnitude from Lot 1 KSD to Lot 3 KSD, i.e., as drug loading increases, thus suggesting reduced interaction between abiraterone and HPBCD in Lots 2 and 3 KSDs. For Lots 4 and 5 KSDs small peak shifts are observed only due to C=C vibrations in B-ring, thus suggestion minimal partial interaction between abiraterone and HPBCD in these KSDs. It can be assumed that there is not much interaction between abiraterone and outer surface of HPBCD in the KSDs due to the hydrophilic nature of HPBCD outersurface. However, there remains a possibility of hydrogen bonding between abiraterone —OH and that of HPBCD when abiraterone is not completely included within HPBCD. This non-included abiraterone has likely less apparent aqueous solubility than abiraterone that is included within HPBCD. Gong and Zhu reported that abiraterone acetate and abiraterone can form complexes with β-cyclodextrin (Gong and Zhu 2013).

Overall, 1D ¹³C ssNMR spectroscopy and Raman spectroscopy suggest that all amorphous abiraterone in Lot 1 KSD is included or complexed within HPBCD; Lots 2 and 3 KSDs contain amorphous abiraterone which is partially complexed within HPBCD; Lots 4 and 5 KSDs contain mostly crystalline abiraterone which has minimal interaction with HPBCD.

The abiraterone:HPBCD molar ratios for Lots 1 to 5 KSDs are 1.0:2.2, 1.0:1.0, 1.0:0.6, 1.0:0.4 and 1.0:0.2 respectively. Thus, as drug loading increases the number of molecules of HPBCD available to interact with abiraterone decreases, thereby leading to reduce interaction between abiraterone and HPBCD. Although the stoichiometry of abiraterone:HPBCD in these KSDs cannot be stated based on 1D ssNMR results, however it appears that a preferred stoichiometry for abiraterone:HPBCD may be 1:2. This can be further substantiated based on theoretical dimensions of abiraterone and HPBCD. The inner cavity diameter of HPBCD is 0.62-0.78 nm, it is partially shielded and its length is 0.79 nm (Szente et al. 2018; Roquette 2006; Tsuchido et al. 2017). Abiraterone is considered as pyridyl derivative of pregnenolone, whose length is reported to be 13 Å, i.e., 1.3 nm (Haider et al. 2010). The kinetic diameter of pyridine is 5.7 Å, i.e., 0.57 nm and that of cyclohexane (similar to A ring—cyclohexanol of abiraterone) is 0.69 nm (Weng et al. 2015). Thus, abiraterone can be included from either ends into HPBCD cavity, but entire length of abiraterone cannot be covered by single molecule of HPBCD. Hence, theoretically for complete abiraterone inclusion, 2 moles of HPBCD are necessary. This conforms with results of solid-state interaction studies, suggesting that 10% drug loaded Lot 1 KSD has complete abiraterone complexation with HPBCD.

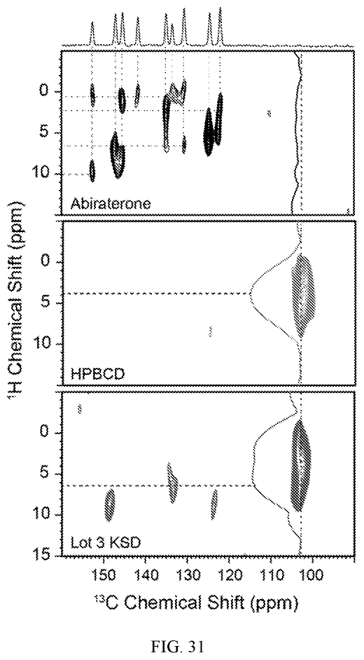

To further investigate the abiraterone-HPBDC interaction at a higher resolution, 2D 13C-1H HETCOR was utilized. Most recently, this 2D heteronuclear correlation spectroscopy has successfully identified the enriched API-polymer interactions in ASDs (Lu et al., Mol Pharm, 2019, 16, 6, 2579-2589; and Hanada et al., International Journal of Pharmaceutics, Volume 548, Issue 1, 5 September 2018, Pages 571-585). For example, different kinds of hydrogen bonding, electrostatic and hydrophobic interactions have been discovered between posaconazole and different polymers including HPMCAS and HPMCP (Lu et al., 2019). In our previous study, higher energy input has been found to enhance the molecular interaction in a ternary ASD (Hanada et al). In the current study, 2D spectra of crystalline abiraterone (black), HPBCD (red) and lot 3 KSD (blue) are shown in FIG. 31. The enlarged spectra in the 13C regions at 90-160 ppm include aromatic peaks of abiraterone at 120-150 ppm and a peak of anomeric carbons of HPBCD at approximately 103 ppm (O'Brein et al., Carbohydrate Research, Volume 339, Issue 1, 2 January 2004, pages 87-96). Abiraterone exhibits well-resolved carbon resonances in the crystalline reference (black) and less number of peaks in the KSD due to line broadening and peak overlapping, which agrees well with the analysis of 1D 13C spectra. HPBCD reference spectrum (red) shows one broad 1H peak which are the protons bonded to the anomeric carbons. Interestingly, the anomeric carbons have a new correlation with a proton peak at approximately 6.4 ppm, which can presumably be assigned to the aromatic protons of abiraterone. This new cross peak suggests intermolecular interaction between the aromatic region of abiraterone and anomeric protons of HPBCD.

### Example 26: Solution State Phase Solubility Profile

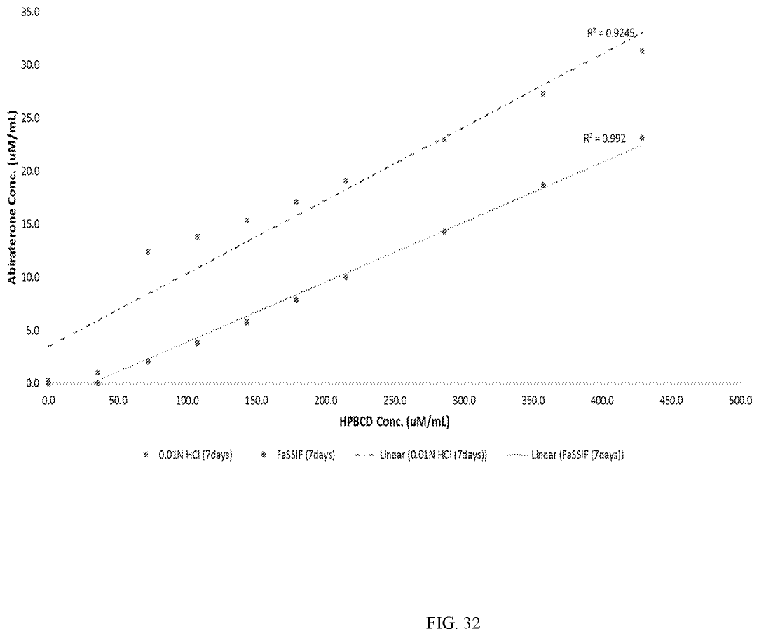

FIG. 32 shows phase solubility profiles for abiraterone-HPBCD in 0.01N HCl and FaSSIF. In both phases, i.e., 0.01N HCl and FaSSIF, as the concentration of HPBCD increased, the solubility of abiraterone increased. The solubility of abiraterone in 0.01N HCl was much higher than that in FaSSIF specifically in HPBCD concentration range of 71.48 μM/mL to 428.88 μM/mL.

Usually, solution state phase solubility profiles are generated to understand drug-CD complexation dynamics for complex preparation through solvent based methods. However, herein we generated them to understand its impact on KSD dissolution. In solution state, CDs can form complexes with free solubilized drug through driving forces such as release of enthalpy-rich water molecules from the CD cavity, van der Waals' interactions, electrostatic interactions, hydrogen bonding and hydrophobic interactions (Loftsson et al. 2005). Based on Higuchi and Connors classification, it can be seen that A-type of solution state phase solubility profiles are generated for abiraterone and HPBCD in both 0.01NHCl and FaSSIF media (FIG. 32) (Loftsson et al. 2005; Saokham et al. 2018). This means that as the concentration of HPBCD increases the amount of abiraterone solubilized increases. Thus, upon KSD dissolution, the re-solubilization of unabsorbed abiraterone would depend on concentration of HPBCD. Hence, highest viable HPBCD concentration is preferred for abiraterone solubilization.

Additionally, higher abiraterone solubilization was seen in 0.01N HCl as compared to FaSSIF because the intrinsic solubility of abiraterone is higher in 0.01N HCl, hence more abiraterone solubilizes and forms complexes with HPBCD in 0.01N HCl as compared to FaSSIF. It should be noted that usually unionized drug forms more stable complexes with CD than ionized drug (Loftsson et al. 2005). Since pKa of abiraterone is 4.81, thus it would form more stable complexes in FaSSIF (Drugbank 2007).

### Example 27: Stability of KSDs

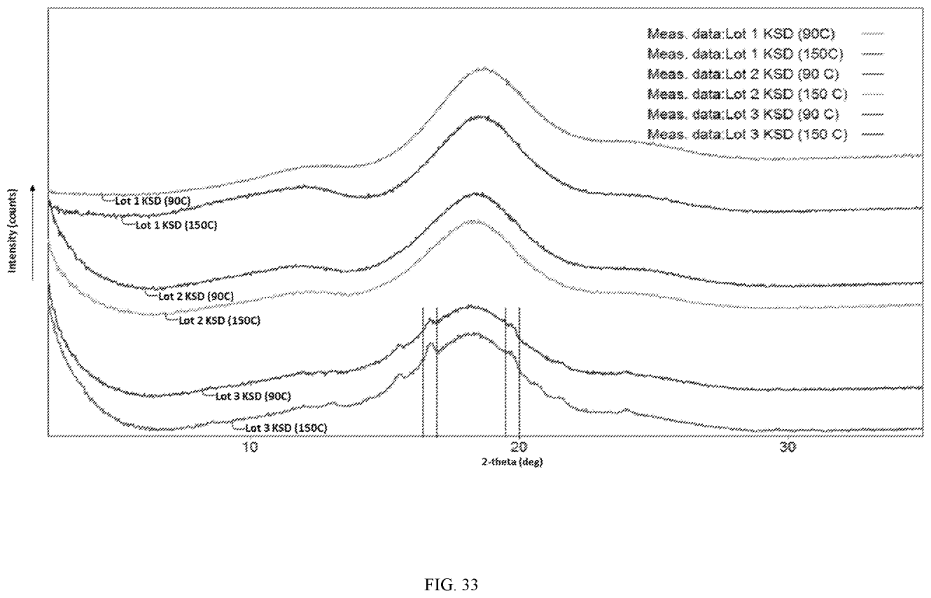

FIG. 33 displays X-ray diffractograms of Lots 1 to 3 KSDs at 90° C. and 150° C. At both 90° C. and 150° C., Lot 1 and 2 KSDs showed a completely halo pattern and no sharp diffraction peaks corresponding to neat abiraterone API (FIG. 28). Lot 3 KSD showed sharp diffraction peaks corresponding to neat abiraterone API (FIG. 28) at both 90° C. and 150° C. Specifically, it showed characteristic abiraterone peaks at 16.65° and 19.66°, but the peak at 8.43° was absent.

We evaluated the stability of KSDs at elevated temperatures. It was seen that abiraterone in Lots 1 and 2 KSDs remained amorphous at both 90° C. and 150° C., whereas Lot 3 KSD showed abiraterone recrystallization at both elevated temperatures (FIG. 33). This could be explained based on solid state interactions between abiraterone and HPBCD in KSDs. Since in both Lots 1 and 2 KSDs each molecule of abiraterone is complexed with at least one molecule of HPBCD, thus abiraterone is thermally and kinetically stabilized in these KSDs inhibiting their recrystallization on heating. In Lot 3 KSDs only a fraction of abiraterone molecules are complexed with HPBCD, thus the free uncomplexed abiraterone molecules recrystallize on heating, thereby destabilizing the KSD.

### Example 28: In Vitro and In Vivo Performance of KSDs

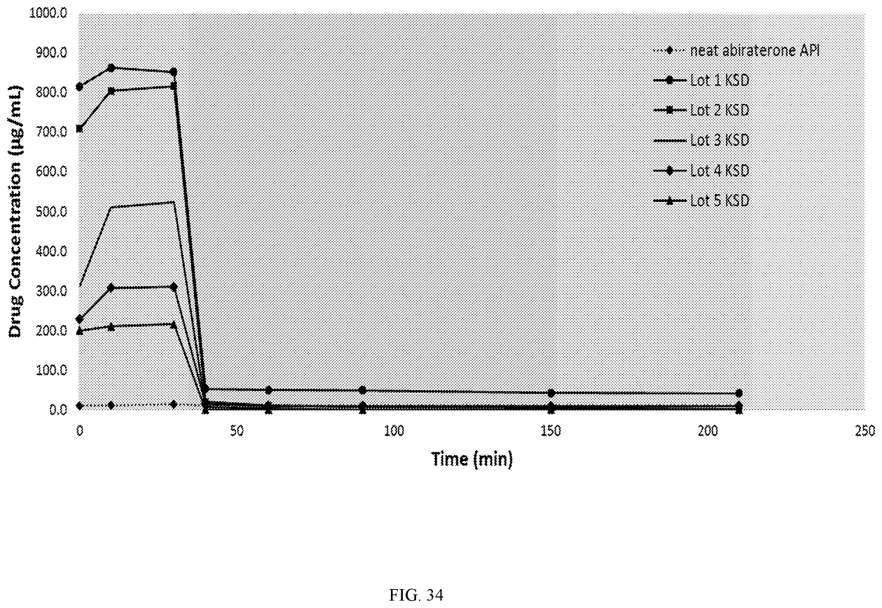

FIG. 34 illustrates, in vitro, non-sink, gastric transfer dissolution profiles of neat abiraterone API and Lots 1 to 5 KSDs; red region (0.01N HCl) and blue region (FaSSIF). Lots 1 to 5 KSDs showed abiraterone dissolution enhancement of 15.7-fold, 12.1-fold, 7.2-fold, 5.0-fold and 3.1-fold respectively, as compared to neat abiraterone API. All KSD lots showed higher abiraterone dissolution in 0.01N HCl as compared to FaSSIF. Amongst KSDs, the abiraterone dissolution enhancement trend was, Lot 1 KSD>Lot 2 KSD>Lot 3 KSD>Lot 4 KSD>Lot 5 KSD. On integrating the total area under the drug dissolution curve (AUDC _{Total}), the relative AUDC _{Total}, for Lots 2 to 5 KSDs was 76.9%, 45.6%, 32.0% and 19.5% respectively, as compared to Lot 1 KSD.

In order to develop a viable dosage form for abiraterone delivery the KSDs were compressed into immediate release tablet formulation. Three tablet formulations containing 10% drug loaded KSD, i.e., Lot 1 KSD, 20% drug loaded KSD, i.e., Lot 2 KSD and 30% drug loaded KSD, i.e., Lot 3 KSD were compressed into Lots 1 Tablet, 2 Tablet and 3 Tablet respectively, containing 50.0 mg of abiraterone. All tablets had optimum hardness, assay, purity, acceptable friability, disintegration time and dissolution. These tablets were tested and compared against Zytiga® tablet containing 250 mg abiraterone acetate.

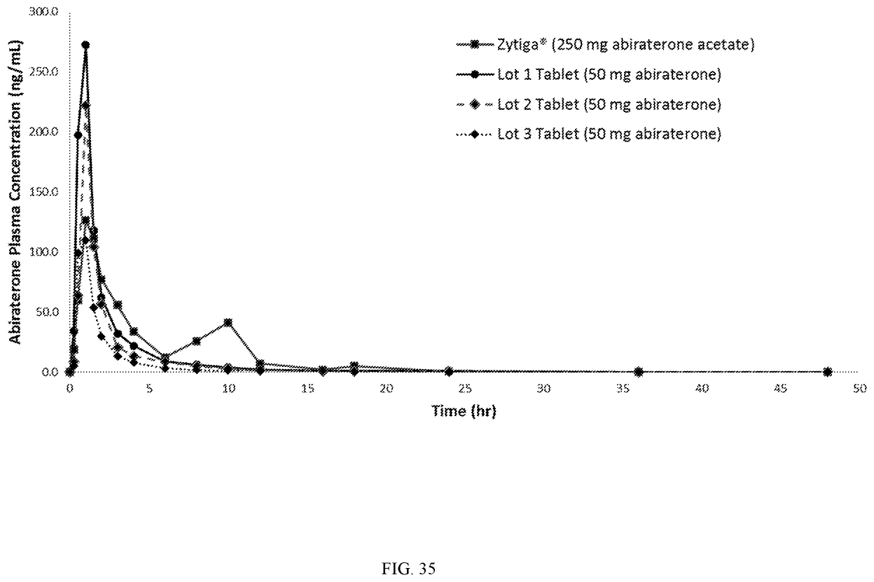

FIG. 35 shows in vivo average plasma concentration v/s time profiles from oral dosing of Zytiga®, Lots 1 to 3 Tablets in fasted non-naïve male beagle dogs and Table 22 lists pharmacokinetic (PK) parameters. Zytiga® showed extremely variable plasma concentration v/s time profile, between all three animals. One of the animal (#JA0167) in Zytiga® study arm (individual animal data not shown) showed, maximum abiraterone plasma concentration, i.e., Cₘₐₓ of 78.50 ng/mL at 1 hr and another Cₘₐₓ of 115.00 ng/mL at 10 hr, contributing to higher drug exposure, i.e., AUC_{(0-48 hr)}for Zytiga®. Lots 1 to 3 tablets showed lower drug exposure variability, i.e., lower %CV for AUC_{(0-48 hr)}as compared to Zytiga®. Zytiga® showed an extremely high variability of 121.39% for Tₘₐₓ. Overall, Lots 1 to 3 tablets were able to enhance the bioavailability of abiraterone by 3.9-fold, 2.7-fold and 1.7-fold respectively, as compared to Zytiga®.

TABLE 22
Results from in vivo pharmacokinetic (PK) study in male beagle dogs

| | | Zytiga ® | | | | | | | |
|---|---|---|---|---|---|---|---|---|---|
| | | (250 mg | | Lot 1 Tablet | | Lot 2 Tablet | | Lot 3 Tablet | |
| | | abiraterone | | (50 mg | | (50 mg | | (50 mg | |
| | | acetate) | | abiraterone) | | abiraterone) | | abiraterone) | |
| | | Average | % CV | Average | % CV | Average | % CV | Average | % CV |
| Cₘₐₓ | ng/mL | 153.00 | 28.24 | 311.67 | 32.61 | 221.90 | 54.72 | 119.87 | 33.77 |
| Tₘₐₓ | hr | 4.17 | 121.39 | 0.83 | 34.64 | 1.00 | 0.00 | 0.83 | 34.64 |
| AUC_{(0-48 hr)} | ng*hr/mL | 523.78 | 46.39 | 451.44 | 32.36 | 319.91 | 42.93 | 195.90 | 35.51 |
| F Value | unitless | 1.0 | | 3.9 | | 2.7 | | 1.7 | |
| (Dose | | | | | | | | | |
| Adjusted) | | | | | | | | | |

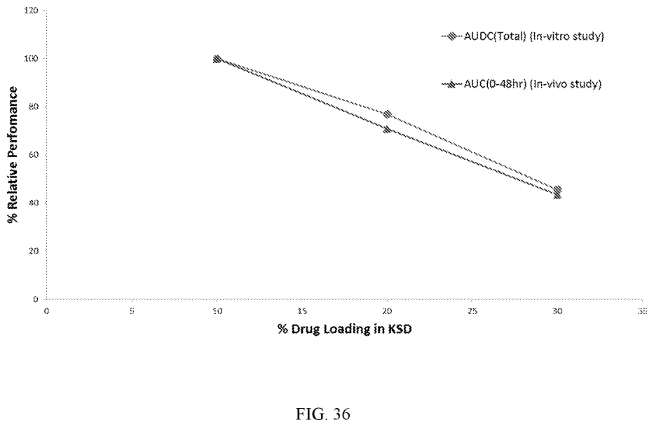

In FIG. 36, we plot the in vitro and in vivo percent relative performance of KSDs with various drug loadings. Both in vitro performance, i.e., in terms of AUDC _{Total} and in vivo performance in terms of AUC_{(0-48 hr)}, decreased as the drug loading in KSDs increased. Interestingly, the relative performance trend for both in vitro and in vivo study was similar.

In the in vitro dissolution study (FIG. 34), it was seen that all the KSDs enhanced the dissolution of abiraterone. The relative in vitro dissolution performance of KSDs decreased as the drug loading increased. This can be attributed to decreased abiraterone-HPBCD complexation, hence reduced abiraterone solubility enhancement with increased drug loading. It should be noted that both increased and decreased dissolution performance is possible with increased drug loading and this is specific to the drug, type of CD, method of preparation and dissolution media (M Badr-Eldin, A Ahmed, and R Ismail, 2013; Semalty et al., 2014; Loh, Tan, and Peh, 2016). As seen in solution state phase solubility profiles, the KSDs also exhibited higher abiraterone dissolution in 0.01NHCl as compared to FaSSIF media. In FaSSIF media the dissolution of Lot 1 KSD was higher as compared to other KSDs, since rate of abiraterone precipitation is lower in Lot 1 KSD due to complete abiraterone complexation. Overall, 10% drug loaded Lot 1 KSD showed highest in vitro dissolution enhancement of abiraterone.

In the in vivo pharmacokinetic study (FIG. 35 and Table 22) it was seen that all the KSD tablets enhanced the exposure of abiraterone as compared to Zytiga® on a dose adjusted basis.

At approximately ⅕^{th} the dose Lots 1 and 2 tablets showed higher Cₘₐₓ than Zytiga®. On a dose adjusted basis, Lots 1, 2 and 3 KSDs showed higher AUC_{(0-48 hr)} than Zytiga® and hence all three KSD tablets were able to enhance the bioavailability of abiraterone. As the drug loading increased in KSDs from Lots 1 to 3 tablets the C., AUC_{(0-48 hr)} and hence bioavailability enhancement decreased. This can be attributed to reduced abiraterone-HPBCD interaction and reduced dissolution with increased drug loading. Also, since Lot 1 tablet contained highest HPBCD amount and yet had highest abiraterone exposure amongst KSD tablets, it can be inferred that HPBCD had no negative effect on abiraterone dissolution. The drug exposure variability reduced with KSD tablets, thereby improving abiraterone pharmacokinetics. Overall, 10% drug loaded Lot 1 KSD showed best in vivo pharmacokinetic performance.

From FIG. 36, it can be seen that in vitro dissolution performance correlated well with in vivo pharmacokinetic performance for KSDs and its tablets. From FIG. 36 it can be extrapolated that 5% drug loaded KSDs could have shown even better performance However, this may not be true since at 10% drug loading abiraterone is completely complexed with HPBCD and additional HPBCD may not show proportional performance enhancement. Additionally, 5% drug loaded KSD would cause a pill burden issue; thus, 10% drug loaded KSD may be optimum.

As shown in Examples 22-28, we developed KSDs with 10 to 50 w/w drug loading and have analyzed these KSDs using X-ray diffractometry and modulated scanning calorimetry. We found that KSDs containing 10 to 30% drug loading were amorphous. Solid state interaction studies using nuclear magnetic resonance spectroscopy and Raman spectroscopy indicated that maximum abiraterone-HPBCD interaction occurred within the 10% drug loaded KSD, this is likely because the molar ratio of abiraterone:HPBCD was 1:2 in this KSD. The solution state phase solubility profile for abiraterone-HPBCD was A-type, meaning that the amount of abiraterone solubilized was directly proportional to the concentration of HPBCD. At elevated temperatures, the 10 and 20% drug loaded KSDs were chemically stable, whereas the 30% drug loaded KSD showed abiraterone recrystallization. As the drug loading increased in the KSD, the in vitro dissolution performance and the in vivo pharmacokinetic performance decreased. This can be attributed to reduced abiraterone-HPBCD interaction with increased drug loading. Overall, 10% drug loaded KSD showed a dissolution enhancement of 15.7-fold as compared to neat abiraterone and bioavailability enhancement of 3.9-fold as compared to commercial abiraterone acetate tablet- Zytiga®. Thus, KinetiSol®, a high energy, solvent free technology, may form an optimally performing 10% abiraterone-HPBCD complex within KSD in terms of improved in vitro and in vivo performance.

Examples 22-28 show that KinetiSol® is as an efficient high energy, solvent free technology to make abiraterone-HPBCD compositions ranging from 10% to 50% drug loading. As drug loading increases the interaction between abiraterone-HPBCD in KSDs decreases, the dissolution enhancement of abiraterone decreases as well as the bioavailability enhancement of abiraterone decreases. The 10% drug loaded KSD is stoichiometrically balanced for complete interaction with abiraterone to entail maximum in vitro and in vivo performance. Thus, 10% drug loaded KSD has the potential for improving therapeutic outcomes in prostate cancer patients.

The above disclosure contains various examples of pharmaceutical formulations, final solid dosage forms, methods of forming pharmaceutical formulations, and methods of administering pharmaceutical formulations. Aspects of these various examples may all be combined with one another, even if not expressly combined in the present disclosure, unless they are clearly mutually exclusive. For example, a specific pharmaceutical formulation may contain amounts of components identified more generally or may be administered in any way described herein.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

### REFERENCES

Various patents, patent applications and publications are cited herein, including those below, the contents of all of which are hereby incorporated in their entireties.

Acharya, M., M. Gonzalez, G. Mannens, R. De Vries, C. Lopez, T. Griffin, and N. Tran. 2013. ‘A phase I, open-label, single-dose, mass balance study of 14C-labeled abiraterone acetate in healthy male subjects’, Xenobiotica, 43: 379-89.

Belfort, Gabriel M.; Harrison, Boyd L. and Botella, GabrielMartinez 2016. ‘Abiraterone Acetate (Zytiga®): An Inhibitor of CYP17 as a Therapeutic for Castration-Resistant Prostate Cancer.’ in, Successful Drug Discovery.

Borba, P. A., M. Pinotti, G. R. Andrade, N. B. da Costa, Jr., L. R. Olchanheski Junior, D. Fernandes, C. E. de Campos, and H. K. Stulzer. 2015. ‘The effect of mechanical grinding on the formation, crystalline changes and dissolution behaviour of the inclusion complex of telmisartan and beta-cyclodextrins’, Carbohydr Polym, 133: 373-83.

Chi, K. N., J. Spratlin, C. Kollmannsberger, S. North, C. Pankras, M. Gonzalez, A. Bernard, H. Stieltjes, L. Peng, J. Jiao, M. Acharya, T. Kheoh, T. W. Griffin, M. K. Yu, C. Chien, and N. P. Tran. 2015. ‘Food effects on abiraterone pharmacokinetics in healthy subjects and patients with metastatic castration-resistant prostate cancer’, J Clin Pharmacol, 55: 1406-14.

COULET, M., M. VAN BORSSUM WAALKES, O. R. LEEUWENKAMP, P. COX, and J. LOHUIS. 2002. ‘Pharmacokinetics of ibafloxacin following intravenous and oral administration to healthy Beagle dogs’, Journal of Veterinary Pharmacology and Therapeutics, 25: 89-97.

Davis, Mark E., and Marcus E. Brewster. 2004. ‘Cyclodextrin-based pharmaceutics: past, present and future’, Nature Reviews Drug Discovery, 3: 1023-35.

Drugbank. 2007. ‘Abiraterone’, Accessed 09/05/2019. https ://www.drugbank.ca/drugs/DB05812.

Ellenberger, Daniel J., Dave A. Miller, and Robert O. Williams. 2018. ‘Expanding the Application and Formulation Space of Amorphous Solid Dispersions with KinetiSol®: a Review’, AAPS PharmSciTech, 19: 1933-56.

FDA, US. 2010. “Clinical pharmacology and biopharmaceutics review(s)—Zytiga®.” In.

FDA, US. 2018. “Highlights of prescribing information- Yonsa®.”

Gala, U, H Pham, and H Chauhan. 2013. ‘Pharmaceutical applications of eutectic mixtures’, J Dev Drug, 2: 1-2.

Gala, Urvi. 2015. ‘Preparation, Characterization and Dissolution Study for Curcumin-Resveratrol-Cyclodextrin Amorphous Ternary System’, Creighton University.

Goldwater, R., A. Hussaini, B. Bosch, and P. Nemeth. 2017. ‘Comparison of a Novel Formulation of Abiraterone Acetate vs. the Originator Formulation in Healthy Male Subjects: Two Randomized, Open-Label, Crossover Studies’, Clin Pharmacokinet, 56: 803-13.

Gong, A., and X. Zhu. 2013. ‘beta-Cyclodextrin sensitized spectrofluorimetry for the determination of abiraterone acetate and abiraterone’, J Fluoresc, 23: 1279-86.

Haider, Shozeb M., Jagdish S. Patel, Chetan S. Poojari, and Stephen Neidle. 2010. ‘Molecular Modeling on Inhibitor Complexes and Active-Site Dynamics of Cytochrome P450 C17, a Target for Prostate Cancer Therapy’, Journal of Molecular Biology, 400: 1078-98.

Huang, Yanbin, and Wei-Guo Dai. 2014. ‘Fundamental aspects of solid dispersion technology for poorly soluble drugs’, Acta Pharmaceutica Sinica B, 4: 18-25.

Hywel Williams, Prashant Agarwal, and Eduardo Jule. 2016. “Abiraterone acetate lipid formulations.” In.

Jug, M., M. Becirevic-Lacan, and S. Bengez. 2009. ‘Novel cyclodextrin-based film formulation intended for buccal delivery of atenolol’, Drug Dev Ind Pharm, 35: 796-807.

Jug, M., and P. A. Mura. 2018. ‘Grinding as Solvent-Free Green Chemistry Approach for Cyclodextrin Inclusion Complex Preparation in the Solid State’, Pharmaceutics, 10.

Knapik-Kowalczuk, Justyna, Krzysztof Chmiel, Karolina Jurkiewicz, Natália T. Correia, Wieslaw Sawicki, and Marian Paluch. 2019. ‘Physical Stability and Viscoelastic Properties of Co-Amorphous Ezetimibe/Simvastatin System’, Pharmaceuticals (Basel, Switzerland), 12: 40.

Li, R., K. Evaul, K. K. Sharma, K. H. Chang, J. Yoshimoto, J. Liu, R. J. Auchus, and N. Sharifi. 2012. ‘Abiraterone inhibits 3beta-hydroxysteroid dehydrogenase: a rationale for increasing drug exposure in castration-resistant prostate cancer’, Clin Cancer Res, 18: 3571-9.

Li, Ying, Zhen-Dan He, Qian-En Zheng, Chengshen Hu, and Wing-Fu Lai. 2018. ‘Hydroxypropyl-β-cyclodextrin for Delivery of Baicalin via Inclusion Complexation by Supercritical Fluid Encapsulation’, Molecules, 23: 1169.

Loftsson, T., and M. E. Brewster. 2012. ‘Cyclodextrins as functional excipients: methods to enhance complexation efficiency’, J Pharm Sci, 101: 3019-32.

Loftsson, T., P. Jarho, M. Masson, and T. Jarvinen. 2005. ‘Cyclodextrins in drug delivery’, Expert Opin Drug Deliv, 2: 335-51.

Loftsson, T., M. D. Moya-Ortega, C. Alvarez-Lorenzo, and A. Concheiro. 2016. ‘Pharmacokinetics of cyclodextrins and drugs after oral and parenteral administration of drug/cyclodextrin complexes’, J Pharm Pharmacol, 68: 544-55.

Loh, Gabriel Onn Kit, Yvonne Tze Fung Tan, and Kok-Khiang Peh. 2016. ‘Enhancement of norfloxacin solubility via inclusion complexation with β-cyclodextrin and its derivative hydroxypropylβ-cyclodextrin’, Asian Journal of Pharmaceutical Sciences, 11: 536-46.

Long, D. A. 2004. ‘Infrared and Raman characteristic group frequencies. Tables and charts George Socrates John Wiley and Sons, Ltd, Chichester, Third Edition, 2001. Price £135’, Journal of Raman Spectroscopy, 35: 905-05.

M Badr-Eldin, Shaimaa, Tarek A Ahmed, and Hatem R Ismail. 2013. ‘Aripiprazole-cyclodextrin binary systems for dissolution enhancement: effect of preparation technique, cyclodextrin type and molar ratio’, Iranian journal of basic medical sciences, 16: 1223-31.

Modekar, Shashikant, and Jagadevappa Patil. 2016. ‘DESIGN, EVALUATION AND CHARACTERIZATION OF SPRAY DRIED CYCLODEXTRIN INCLUSION COMPLEXES OF GLIMEPRIDE’, 5: 1029-46.

Moya-Ortega, Maria D., Martin Messner, Phatsawee Jansook, Thorbørn Terndrup Nielsen, Veronique Wintgens, Kim Lambertsen Larsen, Catherine Amiel, Hákon H. Sigurdsson, and Thorsteinn Loftsson. 2011. ‘Drug loading in cyclodextrin polymers: dexamethasone model drug’, Journal of Inclusion Phenomena and Macrocyclic Chemistry, 69: 377-82.

Mura, P., E. Adragna, A. M. Rabasco, J. R. Moyano, J. I. Pérez-Martìnez, M. J. Arias, and J. M. Ginés. 1999. ‘Effects of the Host Cavity Size and the Preparation Method on the Physicochemical Properties of Ibuproxam-Cyclodextrin Systems’, Drug Development and Industrial Pharmacy, 25: 279-87.

Pessine, FBT, A Calderini, and GL Alexandrino. 2012. ‘Review: cyclodextrin inclusion complexes probed by NMR techniques, magnetic resonance spectroscopy’, Dong-Hyun Kim editor.

Qian, Feng, Jun Huang, and Munir A. Hussain. 2010. ‘Drug-Polymer Solubility and Miscibility: Stability Consideration and Practical Challenges in Amorphous Solid Dispersion Development’, Journal of Pharmaceutical Sciences, 99: 2941-47.

Ramos, Ana I., Teresa M. Braga, Patrícia Silva, José A. Fernandes, Paulo Ribeiro-Claro, Maria de Fátima Silva Lopes, Filipe A. Almeida Paz, and Susana S. Braga. 2013. ‘Chloramphenicol⋅cyclodextrin inclusion compounds: co-dissolution and mechanochemical preparations and antibacterial action’, CrystEngComm, 15: 2822-34.

Ren, ke, Zhi-Rong Zhang, Yanzhen Li, Jie Liu, Dong Zhao, Yi Zhao, and Tao Gong. 2009. ‘Physicochemical characteristics and oral bioavailability of andrographolide complexed with hydroxypropyl-β-cyclodextrin’, Pharmazie, 64: 515-20.

Roquette. 2006. ‘Kleptose Brochure’.

Saokham, P., C. Muankaew, P. Jansook, and T. Loftsson. 2018. ‘Solubility of Cyclodextrins and Drug/Cyclodextrin Complexes’, Molecules, 23.

Savjani, Ketan T., Anuradha K. Gajjar, and Jignasa K. Savjani. 2012. ‘Drug solubility: importance and enhancement techniques’, ISRN pharmaceutics, 2012: 195727-27.

Semalty, Mona, Mitali Panchpuri, Devendra Rawat, and Ajay Semalty. 2014. ‘Cyclodextrin Inclusion Complex of Racecadotril: Effect of Drug-β-Cyclodextrin Ratio and the Method of Complexation’, Current Drug Discovery Technologies, 11: 154-61.

Semcheddine, Farouk, Nida El Islem Guissi, XueYin Liu, ZuoMin Wu, and Bo Wang. 2015. ‘Effects of the Preparation Method on the Formation of True Nimodipine SBE-β-CD/HP-β-CD Inclusion Complexes and Their Dissolution Rates Enhancement’, AAPS PharmSciTech, 16: 704-15.

Sharma, Neha, and Ashish Baldi. 2016. ‘Exploring versatile applications of cyclodextrins: an overview’, Drug Delivery, 23: 729-47.

Solymosi, Tamás, Ferenc Tóth, János Orosz, Orsolya Basa-Dénes, Réka Angi, Tamás Jordán, Zsolt Ötvös, and Hristos Glavinas. 2018. ‘Solubility Measurements at 296 and 310 K and Physicochemical Characterization of Abiraterone and Abiraterone Acetate’, Journal of Chemical & Engineering Data, 63: 4453-58.

Stappaerts, Jef, Sophie Geboers, Jan Snoeys, Joachim Brouwers, Jan Tack, Pieter Annaert, and Patrick Augustijns. 2015. ‘Rapid conversion of the ester prodrug abiraterone acetate results in intestinal supersaturation and enhanced absorption of abiraterone: In vitro, rat in situ and human in vivo studies’, European Journal of Pharmaceutics and Biopharmaceutics, 90: 1-7.

Stolarczyk, E. U., M. Laszcz, A. Les, M. Kubiszewski, K. Kuziak, K. Sidoryk, and K. Stolarczyk. 2018. ‘Design and Molecular Modeling of Abiraterone-Functionalized Gold Nanoparticles’, Nanomaterials (Basel), 8.

Szente, Lajos, Ashutosh Singhal, Andras Domokos, and Byeongwoon Song. 2018. ‘Cyclodextrins: Assessing the Impact of Cavity Size, Occupancy, and Substitutions on Cytotoxicity and Cholesterol Homeostasis’, Molecules, 23: 1228.

Thiry, Justine, Fabrice Krier, Shenelka Ratwatte, Jean-Michel Thomassin, Christine Jerome, and Brigitte Evrard. 2017. ‘Hot-melt extrusion as a continuous manufacturing process to form ternary cyclodextrin inclusion complexes’, European Journal of Pharmaceutical Sciences, 96: 590-97.

Tsuchido, Yuji, Shoji Fujiwara, Takeshi Hashimoto, and Takashi Hayashita. 2017. ‘Development of Supramolecular Saccharide Sensors Based on Cyclodextrin Complexes and Self-assembling Systems’, Chem Pharm Bull (Tokyo), 65: 318-25.

Varma, M. M., and P. S. Kumar. 2012. ‘Formulation and Evaluation of GLZ Tablets Containing PVP K30 and Hydroxyl Propyl Beta Cyclodextrin Solid Dispersion’, International Journal of Pharmaceutical Sciences and Nanotechnology, 5: 1706-19.

Wen, Xianhong, Fei Tan, Zhijun Jing, and Ziuyang Liu. 2004. ‘Preparation and study the 1:2 inclusion complex of carvedilol with β-cyclodextrin’, Journal of Pharmaceutical and Biomedical Analysis, 34: 517-23.

Weng, Yujing, Songbai Qiu, Longlong Ma, Qiying Liu, Mingyue Ding, Qian Zhang, Qi Zhang, and Tiejun Wang. 2015. ‘Jet-Fuel Range Hydrocarbons from Biomass-Derived Sorbitol over Ni-HZSM-5/SBA-15 Catalyst’, Catalysts, 5: 2147-60.

Williams, Hywel; Morgen, Michael; Vodak, David; Jule, Eduardo; Benameur, Hassan. 2019. “M1130-04-28 - Improving Oral Drug Absorption—Which Technology to Select? A Spray-Dried Dispersion and Lipid-Based Formulation Case Study Using Abiraterone Acetate.”

Xu, X. S., C. J. Ryan, K. Stuyckens, M. R. Smith, F. Saad, T. W. Griffin, Y. C. Park, M. K. Yu, P. De Porre, A. Vermeulen, I. Poggesi, and P. Nandy. 2017. ‘Modeling the Relationship Between Exposure to Abiraterone and Prostate-Specific Antigen Dynamics in Patients with Metastatic Castration-Resistant Prostate Cancer’, Clin Pharmacokinet, 56: 55-63.

Xu, X. S., C. J. Ryan, K. Stuyckens, M. R. Smith, F. Saad, T. W. Griffin, Y. C. Park, M. K. Yu, A. Vermeulen, I. Poggesi, and P. Nandy. 2015. ‘Correlation between Prostate-Specific Antigen Kinetics and Overall Survival in Abiraterone Acetate-Treated Castration-Resistant Prostate Cancer Patients’, Clin Cancer Res, 21: 3170-7.

Yuvaraja, K., and Jasmina Khanam 2014. ‘Enhancement of carvedilol solubility by solid dispersion technique using cyclodextrins, water soluble polymers and hydroxyl acid’, Journal of Pharmaceutical and Biomedical Analysis, 96: 10-20.

Zhang, D., Y. C. Lee, Z. Shabani, C. Frankenfeld Lamm, W. Zhu, Y. Li, and A. Templeton. 2018. ‘Processing Impact on Performance of Solid Dispersions’, Pharmaceutics, 10.

1. A pharmaceutical formulation comprising:
abiraterone; and
a cyclic oligomer excipient.

2. The pharmaceutical formulation of claim 1, wherein the abiraterone comprises amorphous abiraterone.

3. The pharmaceutical formulation of claim 2, wherein the abiraterone comprises less than 5% crystalline abiraterone.

4. The pharmaceutical formulation of claim 1, wherein the abiraterone comprises at least 99% abiraterone.

5. The pharmaceutical formulation of claim 1, wherein the abiraterone comprises at least 99% abiraterone, having the structural formula:

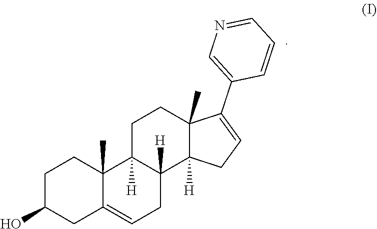

6. The pharmaceutical formulation of claim 1, wherein the abiraterone comprises at least 99% abiraterone salt.

7. The pharmaceutical formulation of claim 1, wherein the abiraterone comprises at least 99% abiraterone ester.

8. The pharmaceutical formulation of claim 7, wherein the abiraterone ester comprises abiraterone acetate, having the structural formula:

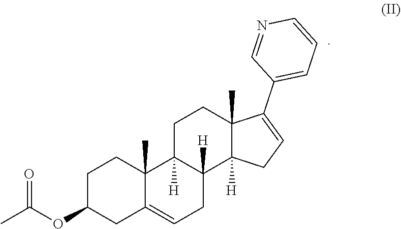

9. The pharmaceutical formulation of claim 1, wherein the abiraterone comprises at least 99% abiraterone solvate.

10. The pharmaceutical formulation of claim 1, wherein the abiraterone comprises at least 99% abiraterone hydrate.

11. (canceled)

12. The pharmaceutical formulation of claim 1, comprising an amount of amorphous abiraterone sufficient to achieve the same or greater therapeutic effect, bioavailability, Cₘᵢₙ, Cₘₐₓ or Tₘₐₓ in a patient as 250 mg, 500 mg or 1000 mg of crystalline abiraterone or crystalline abiraterone acetate when consumed on an empty stomach.

13. The pharmaceutical formulation of claim 1, comprising 50 mg of amorphous abiraterone.

14-16. (canceled)

17. The pharmaceutical formulation of claim 1, wherein the abiraterone and cyclic oligomer are present in a molar ratio of 1:0.25 to 1:25.

18. (canceled)

19. The pharmaceutical formulation of claim 1, comprising 1% to 50% by weight amorphous abiraterone.

20. (canceled)

21. The pharmaceutical formulation of claim 1, wherein the cyclic oligomer excipient comprises a cyclic oligosaccharide or cyclic oligosaccharide derivative.

22-26. (canceled)

27. The pharmaceutical formulation of claim 1 comprising 50% to 99% by weight cyclic oligomer excipient.

28. (canceled)

29. The pharmaceutical formulation of Claim 1 further comprising an additional excipient.

30-42. (canceled)

43. The pharmaceutical formulation of claim 1, further comprising a glucocorticoid replacement API.

44-57. (canceled)

58. A tablet for oral administration comprising any pharmaceutical formulation of claim 1.

59-68. (canceled)

69. A method of forming a pharmaceutical formulation, the method comprising compounding crystalline abiraterone and a cyclic oligomer excipient in a thermokinetic mixer at a temperature less than or equal to 200° C. for less than 300 seconds to form an amorphous abiraterone and a cyclic oligomer excipient.

70-74. (canceled)

75. A method of forming a pharmaceutical formulation, the method comprising hot-melt extrusion processing crystalline abiraterone and a cyclic oligomer excipient to form an amorphous abiraterone and the cyclic oligomer excipient in which the abiraterone is not substantially thermally degraded.

76-79. (canceled)

80. A method of forming a pharmaceutical formulation, the method comprising dissolving crystalline abiraterone and a cyclic oligomer excipient in a common organic solvent to form a dissolved mixture and spray drying the dissolved mixture to form an amorphous abiraterone and cyclic oligomer excipient.

81-85. (canceled)

86. A method of forming a pharmaceutical formulation, the method comprising combining abiraterone and a cyclic oligomer excipient by a method comprising wet mass extrusion, high intensity mixing, high intensity mixing with a solvent, ball milling, or ball milling with a solvent to form an amorphous abiraterone and cyclic oligomer.

87. A method of treating prostate cancer in a patient, the method comprising administering a pharmaceutical formulation of claim 1 to a patient having prostate cancer.

88-99. (canceled)

100. A method of treating breast cancer in a patient, the method comprising administering a pharmaceutical formulation of claim 1 to a patient having breast cancer.

101-110. (canceled)

111. A method of treating salivary gland cancer in a patient, the method comprising administering a pharmaceutical formulation of claim 1 to a patient having salivary gland cancer.

112-121. (canceled)

122. A method of treating cancer in a patient, the method comprising administering a pharmaceutical formulation of claim 1 to a patient having an androgen sensitive cancer.

123-131. (canceled)

132. The pharmaceutical formulation of claim 1, comprising:
an inclusion complex comprising amorphous abiraterone and the cyclic oligomer excipient.

133. The pharmaceutical formulation of claim 132, wherein the pharmaceutical formulation comprises up to 30%, up to 20%, or up to 10% by weight amorphous abiraterone.

134. (canceled)

135. The pharmaceutical formulation of claim 132, wherein the pharmaceutical formulation is formed by a method comprising thermokinetic compounding.

136. The pharmaceutical formulation of claim 132, wherein at least 90%, 91%, 92%, 93%, 94%, 95%, 96%, 97%, 98%, 99%, or 99.9% of the amorphous abiraterone is present in the inclusion complex.

137. The pharmaceutical formulation of claim 132, wherein:
in response to heating the pharmaceutical formulation to a temperature up to 90% of the melting point of a crystalline form of abiraterone, and
allowing the pharmaceutical formulation to cool to room temperature, less than 10%, 9%, 8%, 7%, 6%, 5%, 4%, 3%, 2%, 1%, or 0.1% of the abiraterone is in crystalline form.

138-140. (canceled)
